(19) United States
(12) Patent Application Publication
(10) Pub. No.: US 2025/0066383 A1
(43) Pub. Date: Feb. 27, 2025

(54) PDE 7 MODULATOR COMPOUNDS

(71) Applicant: Omeros Corporation, Seattle, WA (US)

(72) Inventors: Neil S. CUTSHALL, Snohomish, WA (US); Mark A. HADD, Seattle, WA (US); Santosh Kumar KESHIPEDDY, Bellevue, WA (US); Robert H. LEMUS, Seattle, WA (US); Markus METZ, Bellevue, WA (US)

(21) Appl. No.: 18/811,278

(22) Filed: Aug. 21, 2024

Related U.S. Application Data
(60) Provisional application No. 63/578,039, filed on Aug. 22, 2023.

(51) Int. Cl. C07D 495/04; A61K 31/4162; A61K 31/496; A61K 31/5377; C07D 231/16

(52) U.S. Cl. CPC C07D 495/04; A61K 31/4162; A61K 31/496; A61K 31/5377; C07D 231/16

(57) ABSTRACT

Compounds having activity as modulators of phosphodiesterase 7 (PDE 7) are provided. The compounds have the following Structures (I) or (II):

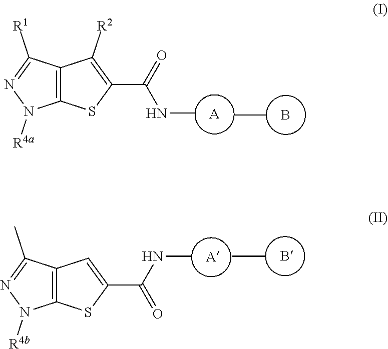

as a stereoisomer, enantiomer, or tautomer thereof or a mixture thereof; or a pharmaceutically acceptable salt, solvate or prodrug thereof, wherein each of R¹, R², R³, R^{4a}, R^{4b}, ring A, ring B, ring A′ and ring B′ (i.e., \textcircled{A}, \textcircled{B}, [symbol] , and [symbol] as shown in Structure (I) and (II), respectively) are as defined herein. Methods associated with preparation and use of such compounds, pharmaceutical compositions comprising such compounds and methods to modulate the activity of phosphodiesterase 7 (PDE 7) are also provided.

### BACKGROUND

### Technical Field

Embodiments of the present disclosure are generally directed to PDE 7 modulator compounds and methods for their preparation and use as therapeutic or prophylactic agents, for example for treatment of addiction and movement disorders.

### Description of the Related Art

It has been suggested that phosphodiesterase 7 (PDE 7) plays an important role for activating T-cells (Beavo, et al., Science, 283, 848 (1999)), and it is well known that activation of T-cells is related to the exacerbation of a variety of allergic diseases, inflammatory diseases, and immunological diseases. Therefore, it is considered that a compound having PDE 7 inhibiting activity would be useful for treating various disease types such as allergic diseases, inflammatory diseases or immunological diseases related to T-cell activity.

Accordingly, there is a need for compounds and compositions that effectively and selectively modulate PDE 7 activity that provide a treatment for diseases and conditions modulated by PDE 7. Embodiments of the present disclosure fulfill this need and provide further related advantages.

### BRIEF SUMMARY

In brief, embodiments of the present disclosure provide compounds, including pharmaceutically acceptable salts, stereoisomers, and prodrugs thereof, which can modulate the activity of PDE 7 (e.g., PDE 7A, PDE 7B, or both).

One embodiment of the disclosure provides compounds of Structure (I):

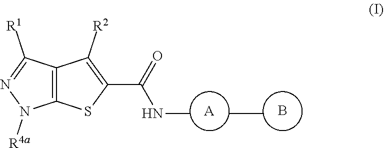

pharmaceutically acceptable salts, stereoisomers, or prodrug thereof, wherein each of R¹, R², R^{4a}, ring A, and ring B (i.e., \textcircled{A} and \textcircled{B}, respectively) are as defined below.

Another embodiment of the disclosure provides compounds of Structure (II):

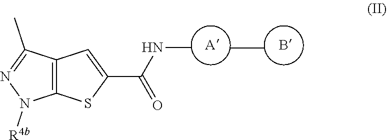

pharmaceutically acceptable salts, stereoisomers, or prodrug thereof, wherein each of R^{4b}, ring A′, and ring B′ (i.e., [symbol] and [symbol] , respectively) are as defined below.

In another aspect, pharmaceutical compositions comprising the disclosed compounds, and methods of use of the same for treatment of neurodegenerative diseases, central nervous system (CNS) disorders, cancer, inflammatory diseases, and combinations thereof are also provided.

In another aspect, pharmaceutical compositions comprising the disclosed compounds, and methods of use of the same for treatment of an addiction including an addiction to an addictive agent, an impulse-control disorder, and an addictive or compulsive behavior.

### DETAILED DESCRIPTION

In the following description, certain specific details are set forth to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is, as “including, but not limited to”.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, the terms “about” and “approximately” mean±20%, ±10%, ±5% or ±1% of the indicated range, value, or structure, unless otherwise indicated. The terms “a” and “an” as used herein refer to “one or more” of the enumerated components. The use of the alternative (e.g., “or”) should be understood to mean either one, both, or any combination thereof of the alternatives.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the features, structures, or characteristics may be combined in any suitable manner in one or more embodiments.

Unless defined otherwise, all technical and scientific terms used herein have the same meaning as is commonly understood by one of skill in the art to which this disclosure belongs. As used in the specification and claims, the singular form “a”, “an” and “the” include plural references unless the context clearly dictates otherwise.

“Amino” refers to the —NH₂ radical.

“Carboxy” or “carboxyl” refers to the —CO₂H radical, which may also exist as a —CO₂⁻ radical depending on the conditions (e.g., pH, counter ion, etc.).

“Cyano” refers to the —CN radical.

“Hydroxy” or “hydroxyl” refers to the —OH radical.

“Alkyl” refers to a saturated, straight or branched hydrocarbon chain radical consisting solely of carbon and hydrogen atoms, having from one to twelve carbon atoms (C₁-C₁₂ alkyl), one to eight carbon atoms (C₁-C₈ alkyl) or one to six carbon atoms (C₁-C₆ alkyl), or any value within these ranges, such as C₄-C₆ alkyl and the like, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl and the like. The number of carbons referred to relates to the carbon backbone and carbon branching but does not include carbon atoms belonging to any substituents. Unless stated otherwise specifically in the specification, an alkyl group is optionally substituted.

“Alkoxy” refers to a radical of the formula —ORₐ where Rₐ is an alkyl radical as defined above containing one to twelve carbon atoms (C₁-C₁₂ alkoxy), one to eight carbon atoms (C₁-C₈ alkoxy) or one to six carbon atoms (C₁-C₆ alkoxy), or any value within these ranges. Unless stated otherwise specifically in the specification, an alkoxy group is optionally substituted.

“Aminyl” refers to a radical of the formula —NRₐR_{b}, where Rₐ and Rb are each independently H or C₁-C₆ alkyl as defined above. When both of Rₐ and R_{b} are H, an “aminyl” group is the same as an “amino” group as defined above. The C₁-C₆ alkyl portion of an aminyl group is optionally substituted unless stated otherwise.

“Aromatic ring” refers to a cyclic planar molecule or portion of a molecule (i.e., a radical) with a ring of resonance bonds that exhibits increased stability relative to other connective arrangements with the same sets of atoms. Generally, aromatic rings contain a set of covalently bound co-planar atoms and comprises a number of π-electrons (for example, alternating double and single bonds) that is even but not a multiple of 4 (i.e., 4n+2 π-electrons, where n=0, 1, 2, 3, etc.). Aromatic rings include, but are not limited to, phenyl, naphthenyl, imidazolyl, pyrrolyl, pyridinyl, pyrimidinyl, pyrazinyl, pyridonyl, pyridazinyl, pyrimidonyl. Unless stated otherwise specifically in the specification, an “aromatic ring” includes all radicals that are optionally substituted.

“Aryl” refers to a carbocyclic ring system radical comprising 6 to 18 carbon atoms, for example 6 to 10 carbon atoms (C₆-C₁₀ aryl) and at least one carbocyclic aromatic ring. For purposes of embodiments of this disclosure, the aryl radical is a monocyclic, bicyclic, tricyclic, or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl group is optionally substituted.

“Carbocyclic” or “carbocycle” refers to a ring system, wherein each of the ring atoms are carbon.

“Cycloalkyl” refers to a non-aromatic monocyclic or polycyclic carbocyclic radical consisting solely of carbon and hydrogen atoms, which may include fused or bridged ring systems, having from three to fifteen ring carbon atoms (C₃-C₁₅ cycloalkyl), from three to ten ring carbon atoms (C₃-C₁₀ cycloalkyl), or from three to eight ring carbon atoms (C₃-C₈ cycloalkyl), or any value within these ranges such as three to four carbon atoms (C₃-C₄ cycloalkyl), and which is saturated or partially unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, (is, 2s, 3s, 4s, 6s, 7s)-cubanyl, and the like. Unless otherwise stated specifically in the specification, a cycloalkyl group is optionally substituted.

“Fused” refers to any ring structure with two or more rings and at least two rings share two adjacent atoms. Unless otherwise stated specifically in the specification, a fused ring group is optionally substituted.

“Bridged” or refers to any ring structure described herein having two or more joined rings that share three or more atoms and can be carbocyclic (i.e., all ring atoms are carbons) or heterocyclic (i.e., the rings comprise carbon and one or more heteroatoms). Unless otherwise stated specifically in the specification, a bridged ring group is optionally substituted.

“Spiro” refers to any ring structure described herein having at least 2 molecular rings with only one common atom. Unless otherwise stated specifically in the specification, a spiro ring group is optionally substituted.

“Halo” refers to bromo, chloro, fluoro, or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group is optionally substituted.

“Halocycloalkyl” refers to a cycloalkyl radical, as defined above, that is substituted by one or more halo radicals, as defined above, e.g., trifluorocyclopropyl, difluorocyclobutyl, trichlorocyclopentyl, 2,2,2-trifluorocyclohexyl, 1,2-difluorocyclopropyl, 3-bromo-2-fluorocyclopropyl, 1,2-dibromocyclopentyl, and the like. Unless stated otherwise specifically in the specification, a halocycloalkyl group is optionally substituted.

“Hydroxylalkyl” refers to an alkyl radical, as defined above that is substituted by one or more hydroxyl radical. The hydroxyalkyl radical is joined at the main chain through the alkyl carbon atom. Unless stated otherwise specifically in the specification, a hydroxylalkyl group is optionally substituted.

“Alkylaminyl” refers to a radical of the formula —NHRₐ or —NRₐRₐ where each Rₐ is, independently, an alkyl radical as defined above containing one to twelve carbon atoms. A “haloalkylaminyl” group is an alkylaminyl group comprising at least one halo substituent on the alkyl group. A “hydroxylalkylaminyl” group is an alkylaminyl group comprising at least one hydroxyl substituent on the alkyl group. An “amidinylalkylaminyl” group is an alkylaminyl group comprising at least one amidinyl substituent on the alkyl group. Unless stated otherwise specifically in the specification, an alkylaminyl, haloalkylaminyl, hydroxylalkylaminyl, and/or amidinylalkylaminyl group is optionally substituted.

“Alkylaminylalkyl” refers to an alkyl group comprising at least one alkylaminyl substituent. The alkylaminyl substituent can be on a tertiary, secondary, or primary carbon. Unless stated otherwise specifically in the specification, an alkylaminylalkyl group is optionally substituted.

“Alkylcarbonyl” refers to a radical of the formula —C(═O)Rₐ where Rₐ is an alkyl radical as defined above containing one to twelve carbon atoms. Unless stated otherwise specifically in the specification, an alkylcarbonyl group is optionally substituted.

“Haloalkylcarbonyl” refers to a radical of the formula —C(═O)Rₐ where Rₐ is an haloalkyl radical as defined above containing one to twelve carbon atoms. Unless stated otherwise specifically in the specification, a haloalkylcarbonyl group is optionally substituted.

“Cycloalkylcarbonyl” refers to a radical of the formula —C(═O)Rₐ where Rₐ is cycloalkyl as defined above. Unless stated otherwise specifically in the specification, a cycloalkylcarbonyl group is optionally substituted.

“Halocycloalkylaminyl” refers to a radical of the formula —NRₐR_{b}, where Rₐ and R_{b} are each independently H or C₁-C₆ alkyl and the other comprises a halocycloalkyl radical as defined above. The C₁-C₆ alkyl and/or halocycloalkyl portion of a halocycloalkylaminyl group is optionally substituted unless stated otherwise.

“Alkylcarbonylaminyl” refers to a radical of the formula —NRₐR_{b} where Rₐ is H or C₁-C₆ alkyl and R_{b} is an alkylcarbonyl radical as defined above. The C₁-C₆ alkyl and/or alkylcarbonyl portion of an alkylcarbonylaminyl group is optionally substituted unless stated otherwise.

“Haloalkylcarbonylaminyl” refers to a radical of the formula —NRₐR_{b} where Rₐ is H or C₁-C₆ alkyl and R_{b} is a haloalkylcarbonyl radical as defined above. The C₁-C₆ alkyl and/or haloalkylcarbonyl portion of a haloalkylcarbonylaminyl group is optionally substituted unless stated otherwise.

“Cycloalkylcarbonylaminyl” refers to a radical of the formula —NRₐR_{b} where Rₐ is H or C₁-C₆alkyl and R_{b} is a cycloalkylcarbonyl radical as defined above. The C₁-C₆ alkyl and/or cycloalkylcarbonyl portion of a cycloalkylcarbonylaminyl group is optionally substituted unless stated otherwise.

“Heterocyclyl” refers to a 3- to 18-membered, for example 3- to 10-membered or 3- to 8-membered, non-aromatic ring radical having one to ten ring carbon atoms (e.g., two to ten) and from one to six ring heteroatoms selected from the group consisting of nitrogen, oxygen, and sulfur. Unless stated otherwise specifically in the specification, the heterocyclyl radical is partially or fully saturated and is a monocyclic, bicyclic, tricyclic, or tetracyclic ring system, which may include fused, spirocyclic and/or bridged ring systems. Nitrogen and sulfur atoms in a heterocyclyl radical are optionally oxidized, and nitrogen atoms may be optionally quaternized. Examples of such heterocyclyl radicals include, but are not limited to, dioxolanyl, thienyl[1,3]dithianyl, decahydroisoquinolyl, furanonyl, imidazolinyl, imidazolidinyl, isothiazolidinyl, isoxazolidinyl, morpholinyl, octahydroindolyl, octahydroisoindolyl, hexahydro-1H-pyrrolizine, 2-oxopiperazinyl, 2-oxopiperidinyl, 2-oxopyrrolidinyl, oxazolidinyl, oxiranyl, piperidinyl, piperazinyl, 4-piperidonyl, azetidinyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxaspiro[3.3]heptanyl, and 1,1-dioxo-thiomorpholinyl. Unless stated otherwise specifically in the specification, a heterocyclyl group is optionally substituted.

“Heterocyclylalkyl” refers to an alkyl group comprising at least one heterocyclyl substituent. Unless stated otherwise specifically in the specification, a heterocyclylalkyl group is optionally substituted.

“Heteroaryl” refers to a 5- to 18-membered, for example 5- to 6-membered, ring system radical comprising one to thirteen ring carbon atoms, one to six ring heteroatoms selected from the group consisting of nitrogen, oxygen, and sulfur, and at least one aromatic ring. Heteroaryl radicals may be a monocyclic, bicyclic, tricyclic, or tetracyclic ring system, which may include fused or bridged ring systems; and the nitrogen, carbon, or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, heteroaryl groups are optionally substituted.

The term “substituted” as used herein means any of the above groups (e.g., alkyl, alkenyl, alkylene, alkylcarbonyl, alkoxy, alkoxyalkyl, aminylalkyl, aryl, cyanoalkyl, cycloalkyl, haloalkyl, heterocyclyl, heterocyclene, heterocyclylalkyl, heteroaryl, heteroarylalkyl and/or hydroxylalkyl) wherein at least one hydrogen atom (e.g., 1, 2, 3 or all hydrogen atoms) is replaced by a bond to a non-hydrogen substituent. Examples of non-hydrogen substituents include, but are not limited to amino, carboxyl, cyano, hydroxyl, halo, nitro, oxo, thiol, thioxo, alkyl, alkenyl, alkylcarbonyl, alkoxy, aryl, cyanoalkyl, cycloalkyl, haloalkyl, heterocyclyl, heterocyclylalkyl, heteroaryl, heteroarylalkyl and/or hydroxylalkyl substituents, each of which may also be optionally substituted with one or more of the above substituents.

In some specific embodiments, the optional substitutions are independently selected from the group consisting of halo, C₁-C₆ alkyl and C₁-C₆ alkoxy. In other embodiments, the optional substituents are independently selected from the group consisting of amino, halo, hydroxyl, oxo, cyano, C₁-C₆ alkyl, C₁-C₆ haloalkyl, C₁-C₆ hydroxylalkyl, C₁-C₆ aminoalkyl, C₁-C₆ alkylaminylalkyl, C₁-C₆ alkoxy; C₁-C₆ haloalkoxy C₁-C₆ alkylcarbonyl, C₁-C₆ haloalkylcarbonyl, C₁-C₆ alkylaminyl, C₁-C₆ haloalkylaminyl, C₁-C₆ alkylcarbonylaminyl, C₁-C₆ haloalkylcarbonylaminyl, C₃-C₈ cycloalkyl, C₃-C₈ cycloalkylcarbonyl, C₃-C₈ halocycloalkylaminyl, and C₃-C₈ cycloalkylcarbonylaminyl. In some more specific embodiments, the optional substituents are independently selected from the group consisting of halo, cyano, C₁-C₆ alkyl, and C₁-C₆ haloalkyl.

The term “effective amount” or “therapeutically effective amount” refers to that amount of a compound described herein that is sufficient to affect the intended application including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the compounds chosen, the dosing regimen to be followed, whether it is administered in combination with other compounds, timing of administration, the tissue to which it is administered, and the physical delivery system in which it is carried.

As used herein, “treatment” or “treating” refer to an approach for obtaining beneficial or desired results with respect to a disease, disorder or medical condition including but not limited to a therapeutic effect and/or a prophylactic effect. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. A prophylactic effect includes delaying or eliminating the appearance of a disease or condition, delaying, or eliminating the onset of symptoms of a disease or condition, slowing, halting, or reversing the progression of a disease or condition, or any combination thereof. In certain embodiments, for prophylactic benefit, the compositions are administered to a subject at risk of developing a particular disease, or to a subject reporting one or more of the physiological symptoms of a disease, even though a diagnosis of this disease may not have been made.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

“Pharmaceutically acceptable salt” includes both acid and base addition salts.

“Pharmaceutically acceptable acid addition salt” refers to those salts which retain the biological effectiveness of the free bases, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable acid addition salts are those that are pharmacologically effective and suitable for contact with the tissues of patients without undue toxicity, irritation, or allergic response. Pharmaceutically acceptable acid addition salts which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness of the free acids, which are biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable base addition salts are those that are pharmacologically effective and suitable for contact with the tissues of patients without undue toxicity, irritation, or allergic response. Pharmaceutically acceptable base addition salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

In some embodiments, pharmaceutically acceptable salts include quaternary ammonium salts such as quaternary amine alkyl halide salts (e.g., methyl bromide).

“Subject” refers to an animal, such as a mammal, for example a human. The methods described herein can be useful in both human therapeutics and veterinary applications. In some embodiments, the subject is a mammal, and in some embodiments, the subject is human.

“Mammal” includes humans and both domestic animals such as laboratory animals and household pets (e.g., cats, dogs, swine, cattle, sheep, goats, horses, rabbits), and non-domestic animals such as wildlife and the like.

“Prodrug” is meant to indicate a compound that may be converted under physiological conditions or by solvolysis to a biologically active compound described herein (e.g., compounds of Structure (I) or (II)). Thus, the term “prodrug” refers to a precursor of a biologically active compound that is pharmaceutically acceptable. In some embodiments, a prodrug is inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or thiol group is bonded to any group that, when the prodrug of the active compound is administered to a mammalian subject, cleaves to form a free hydroxy, free amino or free mercapto group, respectively. Examples of prodrugs include, but are not limited to, acetate, formate, and benzoate derivatives of a hydroxy functional group, or acetamide, formamide and benzamide derivatives of an amine functional group in the active compound and the like.

The term “in vivo” refers to an event that takes place in a subject's body.

Embodiments disclosed herein are also meant to encompass all pharmaceutically acceptable compounds of Structure (I) or (II).

Certain embodiments are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, embodiments include compounds produced by a process comprising administering a compound of this disclosure to a mammal for a period sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood, or other biological samples.

“Stable compound” and “stable structure” are meant to indicate a compound that is sufficiently robust to survive isolation to a useful degree of purity from a reaction mixture, and formulation into an efficacious therapeutic agent.

Often crystallizations produce a solvate of the compounds disclosed herein. As used herein, the term “solvate” refers to an aggregate that comprises one or more compounds of the disclosure with one or more molecules of solvent. In some embodiments, the solvent is water, in which case the solvate is a hydrate. Alternatively, in other embodiments, the solvent is an organic solvent. Thus, the compounds of the present disclosure may exist as a hydrate, including a monohydrate, dihydrate, hemihydrate, sesquihydrate, trihydrate, tetrahydrate and the like, as well as the corresponding solvated forms. In some embodiments, the compounds of the disclosure are a true solvate, while in other cases, the compounds of the disclosure merely retain adventitious water or is a mixture of water plus some adventitious solvent.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

A “pharmaceutical composition” refers to formulations of compounds of the disclosure and a medium generally accepted in the art for the delivery of compounds of the disclosure to mammals, e.g., humans. Such a medium includes all pharmaceutically acceptable carriers, diluents or excipients therefor.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

The compounds of the disclosure (i.e., compounds of Structure (I) or (II)) or their pharmaceutically acceptable salts may contain one or more centers of geometric asymmetry and may thus give rise to stereoisomers such as enantiomers, diastereomers, and other stereoisomeric forms that are defined, in terms of absolute stereochemistry, as (R)- or (S)- or, as (D)- or (L)- for amino acids. Embodiments thus include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Embodiments of the present disclosure include all manner of rotamers and conformationally restricted states of a compound of the disclosure. Atropisomers, which are stereoisomers arising because of hindered rotation about a single bond, where energy differences due to steric strain or other contributors create a barrier to rotation that is high enough to allow for isolation of individual conformers, are also included. As an example, certain compounds of the disclosure may exist as mixtures of atropisomers or purified or enriched for the presence of one atropisomer.

In some embodiments, the compounds of Structure (I) or (II) are a mixture of enantiomers or diastereomers. In other embodiments, the compounds of Structure (I) or (II) are substantially one enantiomer or diastereomer.

A “tautomer” refers to a proton shift from one atom of a molecule to another atom of the same molecule. Embodiments thus include tautomers of the disclosed compounds.

The chemical naming protocol and structure diagrams used herein are a modified form of the I.U.P.A.C. nomenclature system, using the ACD/Name Version 9.07 software program and/or ChemDraw Professional Version 17.0.0.206 software naming program (CambridgeSoft). For complex chemical names employed herein, a substituent group is typically named before the group to which it attaches. For example, cyclopropylethyl comprises an ethyl backbone with a cyclopropyl substituent. Except as described below, all bonds are identified in the chemical structure diagrams herein, except for all bonds on some carbon atoms, which are assumed to be bonded to sufficient hydrogen atoms to complete the valency.

### Compounds

The disclosure provides compounds including pharmaceutically acceptable salts, stereoisomers, and prodrugs thereof, which are capable of modulating PDE 7A, 7B, or both.

Accordingly, one embodiment provides a compound of Structure (I):

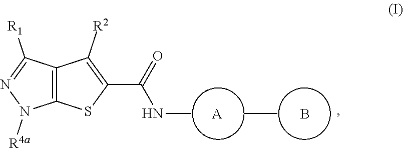

wherein:
ring A is optionally substituted C₃-C₆ cycloalkyl;
ring B is optionally substituted C₄-C₈ cycloalkyl, optionally substituted C₆-C₁₀ aryl, or optionally substituted 4-10 membered heterocyclyl;
R¹ is hydrogen or optionally substituted C₁₋₃ alkyl;
R² is hydrogen, optionally substituted C₁₋₃ alkyl, or halo; and
R^{4a} is optionally substituted phenyl or optionally substituted 5-10 membered heteroaryl; provided that:
when R^{4a} is unsubstituted phenyl, then ring A is C₅-C₆ cycloalkyl and ring B optionally substituted C₄-C₈ cycloalkyl, optionally substituted C₆-C₁₀ aryl, or optionally substituted 6-10 membered heterocyclyl,
as a stereoisomer, enantiomer, or tautomer thereof or a mixture thereof; or a pharmaceutically acceptable salt, solvate, or prodrug thereof.

In some embodiments, when R^{4a} is unsubstituted phenyl, then ring A is not unsubstituted cyclobutyl. In certain embodiments, when R^{4a} is unsubstituted phenyl, then ring B is not pyrrolidinyl-2,5-dione.

In certain embodiments, R¹ is hydrogen. In some embodiments, R¹ is unsubstituted C₁₋₃ alkyl. In some embodiments, R¹ is unsubstituted methyl, ethyl, n-propyl, or isopropyl. In some embodiments, R¹ is methyl. In certain embodiments, R¹ is ethyl. In some embodiments, R¹ is n-propyl. In some embodiments, R¹ is isopropyl.

In some embodiments, R² is hydrogen. In some embodiments, R² is unsubstituted C₁₋₃ alkyl. In some embodiments, R² is halo. In some embodiments, R² is fluoro, chloro, or bromo. In certain embodiments, R² is fluoro. In certain embodiments, R² is unsubstituted methyl, ethyl, n-propyl, or isopropyl.

In some embodiments of Structure (I), the compound has one of the following Structures (IB) or (IC):

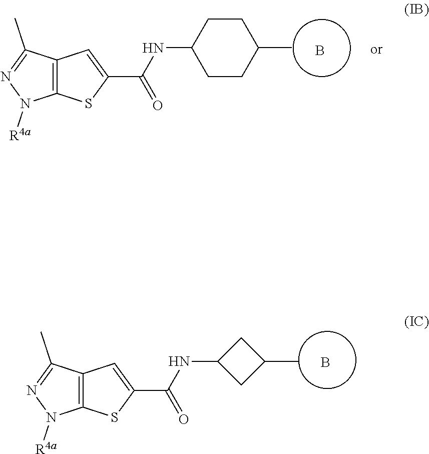

as a stereoisomer, enantiomer, or tautomer thereof or a mixture thereof; or a pharmaceutically acceptable salt, solvate, or prodrug thereof.

In some embodiments, ring B is optionally substituted 4-10 membered heterocyclyl. In some embodiments, ring B is an optionally substituted 4-membered heterocyclyl. In certain embodiments, ring B is an optionally substituted 5-membered heterocyclyl. In some embodiments, ring B is an optionally substituted 6-membered heterocyclyl.

In some embodiments, ring B comprises at least one ring nitrogen. In some embodiments, ring B comprises at least two ring nitrogens. In certain embodiments, ring B comprises at least one ring oxygen. In some embodiments, ring B comprises at least one ring nitrogen and at least one ring oxygen.

In some embodiments, ring B is optionally substituted morpholino, piperidinyl, piperazinyl, or azetidinyl. In some embodiments, the 4-10 membered heterocyclyl is optionally substituted with one or more substituents selected from the group consisting of C₁-C₃ alkyl, C₁-C₃ alkoxy, C₁-C₃ haloalkyl, C₁-C₃ haloalkoxy, C₃-C₈ cycloalkyl, or —C(═O)—C₁-C₃ alkyl.

In some embodiments, ring B is optionally substituted morpholino, piperidinyl, piperazinyl, or azetidinyl. In some embodiments, the 4-10 membered heterocyclyl is optionally substituted with one or more substituents selected from the group consisting of C₁-C₃ alkyl (e.g., methyl, ethyl, n-propyl, or isopropyl), C₁-C₃ alkoxy (e.g., methoxy, ethoxy, n-propoxy, or iso-propoxy), C₁-C₃ haloalkyl, halo, cyano, C₁-C₃ haloalkoxy, C₃-C₈ cycloalkyl (e.g., cyclopropyl, cyclobutyl, cyclopentyl, cyclohexyl, etc.), —C(═O)—C₁-C₃ alkyl, —C(═S)—C₁-C₃ alkyl, or —C(═O)—N(C₁-C₃ alkyl)₂.

In some embodiments, the 4-10 membered heterocyclyl is substituted with one iso-propyl substituent, one or two halo substituents, one difluoromethyl substituent, one methoxy substituent, one difluoromethoxy substituent, one trifluoroethyl substituent, one difluoroethyl substituent, one —C(═O)—CH₃ substituent, one —C(═S)—CH₃ substituent, one cyano substituent, one cyclopropyl substituent (unsubstituted), or one —C(═O)—N(CH₃)₂ substituent.

In some embodiments, the 4-10 membered heterocyclyl is unsubstituted.

In certain embodiments, ring B has one of the following structures:

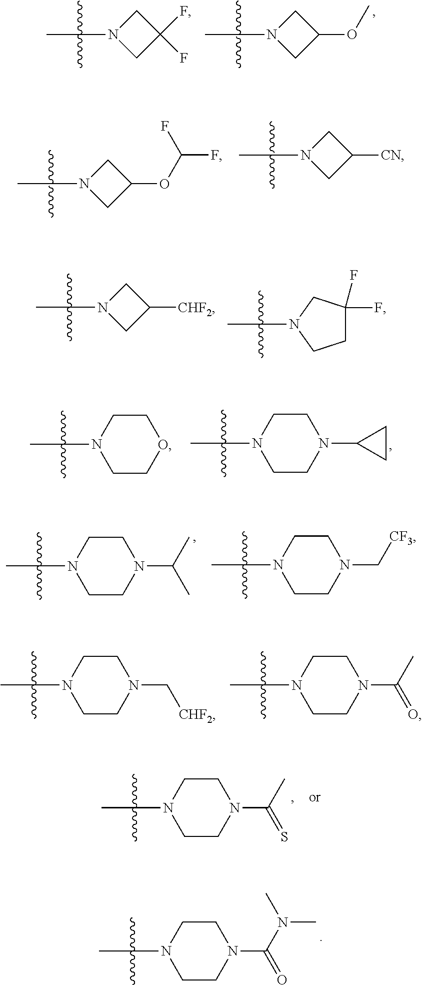

In some embodiments, ring B has one of the following structures:

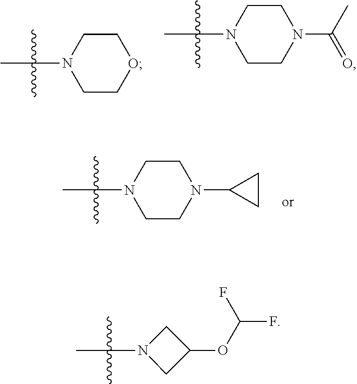

In certain embodiments, R^{4a} is optionally substituted with one or more substituents selected from C₁-C₃ alkyl, C₁-C₃ alkoxy, C₁-C₃ haloalkyl, C₁-C₃ haloalkoxy, C₃-C₈ cycloalkyl, halo, and cyano. In some embodiments, R^{4a} is substituted with one halo substituent. In certain embodiments, R^{4a} is substituted with one chloro substituent.

In some embodiments, R^{4a} has one of the following structures:

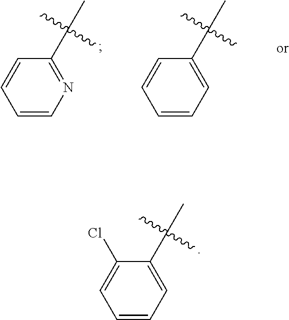 [SMILES: *c1ccccn1 ; *c1ccccc1 ; *c1ccccc1Cl]

In some embodiments, R^{4a} has the following structure:

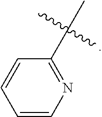 [SMILES: *c1ccccn1]

In some embodiments, R^{4a} has the following structure:

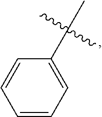 [SMILES: *c1ccccc1]

In some embodiments, R^{4a} has the following structure:

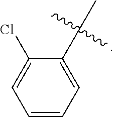 [SMILES: *c1ccccc1Cl]

In some embodiments, the compound is a free base form. In certain embodiments, the compound is a pharmaceutically acceptable salt. In some embodiments, the compound is a trifluoroacetic acid salt. In some embodiments, the compound is a hydrochloric acid salt. In some embodiments, the compound is a formic acid salt. In certain embodiments, the compound is a tautomer.

In various embodiments, the compound has one of the structures set forth in Table 1 below, as a stereoisomer, enantiomer, or tautomer thereof or a mixture thereof; or a pharmaceutically acceptable salt, solvate, or prodrug thereof. Compounds in Table 1 were prepared as described in the Examples or methods known in the art and analyzed by mass spectrometry and/or ¹H NMR spectroscopy.

TABLE 1
Representative Compounds of Structure (I)

| No. | Structure | Name | Mass^{†} |
|---|---|---|---|
| I-1 | 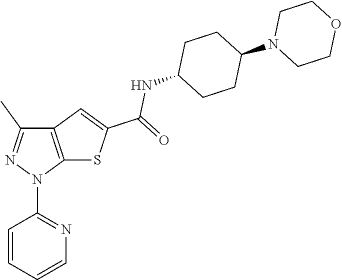 [SMILES: Cc1nn(-c2ccccn2)c2sc(C(=O)N[C@H]3CC[C@H](N4CCOCC4)CC3)cc12] | 3-methyl-N-((1r,4r)-4- morpholinocyclohex- yl)-1-(pyridin-2-yl)- 1H-thieno[2,3-c]pyr- azole-5-carboxamide | 426.67 |
| I-2^{‡} | 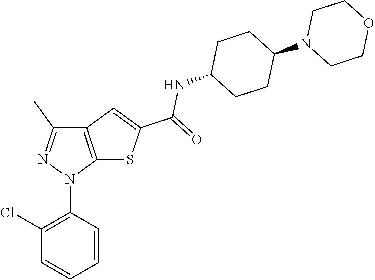 [SMILES: Cc1nn(-c2ccccc2Cl)c2sc(C(=O)N[C@H]3CC[C@H](N4CCOCC4)CC3)cc12] | 1-(2-chlorophenyl)-3- methyl-N-((1r,4r)-4- morpholinocyclohex- yl)-1H-thieno[2,3-c] pyrazole-5-carbox- amide | 459.65 |
| I-3 | 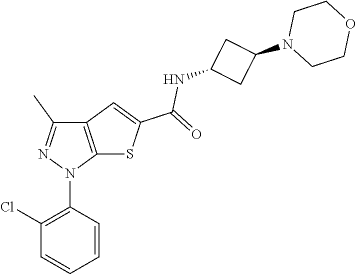 [SMILES: Cc1nn(-c2ccccc2Cl)c2sc(C(=O)N[C@H]3C[C@H](N4CCOCC4)C3)cc12] | 1-(2-chlorophenyl)-3- methyl-N-((1r,3r)-3- morpholinocyclobut- yl)-1H-thieno[2,3-c] pyrazole-5-carbox- amide | 431.57 |
| I-4 | 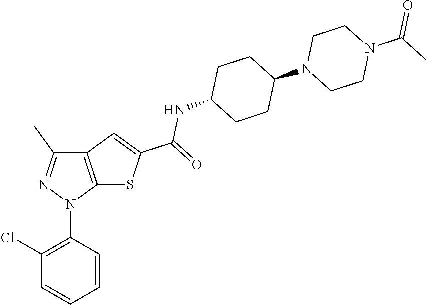 [SMILES: CC(=O)N1CCN([C@H]2CC[C@H](NC(=O)c3cc4c(C)nn(-c5ccccc5Cl)c4s3)CC2)CC1] | N-((1r,4r)-4-(4-acetyl- piperazin-1-yl)cyclo- hexyl)-1-(2-chloro- phenyl)-3-methyl-1H- thieno[2,3-c]pyrazole- 5-carboxamide | 500.63 |
| I-5 | 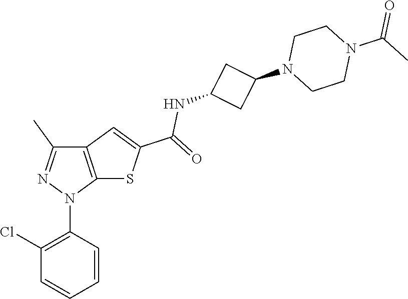 [SMILES: CC(=O)N1CCN([C@H]2C[C@H](NC(=O)c3cc4c(C)nn(-c5ccccc5Cl)c4s3)C2)CC1] | N-((1r,3r)-3-(4-acetyl- piperazin-1-yl)cyclo- butyl)-1-(2-chlorophen- yl)-3-methyl-1H-thieno [2,3-c]pyrazole-5- carboxamide | 472.65 |
| I-6^{‡} | 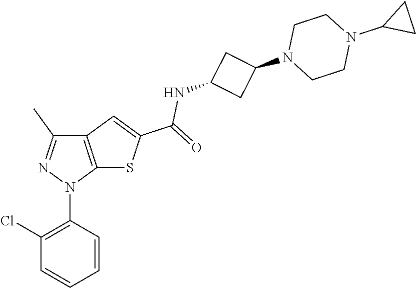 [SMILES: Cc1nn(-c2ccccc2Cl)c2sc(C(=O)N[C@H]3C[C@H](N4CCN(C5CC5)CC4)C3)cc12] | 1-(2-chlorophenyl)-N- ((1r,3r)-3-(4-cyclo- propylpiperazin-1-yl)- cyclobutyl)-3-methyl- 1H-thieno[2,3-c]pyr- azole-5-carboxamide | 470.65 |
| I-7 | 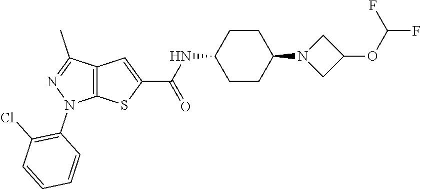 [SMILES: Cc1nn(-c2ccccc2Cl)c2sc(C(=O)N[C@H]3CC[C@H](N4CC(OC(F)F)C4)CC3)cc12] | 1-(2-chlorophenyl)-N- ((1r,4r)-4-(3-(difluoro- methoxy)azetidin-1-yl)- cyclohexyl)-3-methyl- 1H-thieno[2,3-c]pyr- azole-5-carboxamide | 495.54 |
| I-8 | 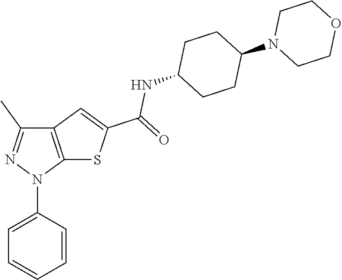 [SMILES: Cc1nn(-c2ccccc2)c2sc(C(=O)N[C@H]3CC[C@H](N4CCOCC4)CC3)cc12] | 3-methyl-N-((1r,4r)-4- morpholinocyclohex- yl)-1-phenyl-1H-thieno [2,3-c]pyrazole-5- carboxamide | 425.57 |

^{†}Observed mass: Positive Electrospray [M + H]⁺
^{‡}Also obtained as a trifluoroacetate salt

One embodiment provides a compound of Structure (II):

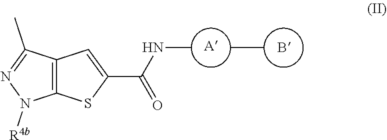

wherein:
ring A′ is optionally substituted C₃-C₈ cycloalkyl, or has one of the following structures:

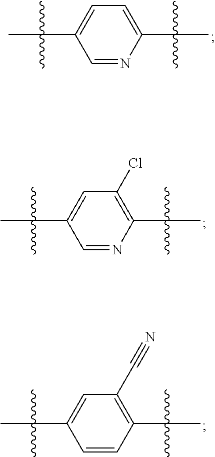 [SMILES: *c1ccc(*)nc1 ; *c1cnc(*)c(Cl)c1 ; *c1ccc(*)c(C#N)c1]

ring B′ is optionally substituted 4-10 membered heterocyclyl; and
R^{4b} is —CH₂CH(CH₃)₂, —CH₂C(CH₃)₃

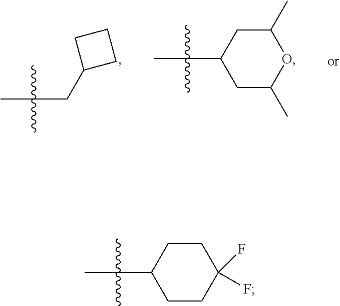

provided that:
when R^{4b} is —CH₂CH(CH₃)₂ and ring A′ has the following structure:

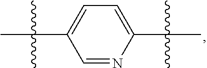 [SMILES: *c1ccc(*)nc1]

then ring B′ is not piperidinyl or 2,6-dimethyl-4-morpholinyl,
as a stereoisomer, enantiomer, or tautomer thereof or a mixture thereof; or a pharmaceutically acceptable salt, solvate, or prodrug thereof.

In some embodiments, R^{4b} is —CH₂CH(CH₃)₂ or —CH₂C(CH₃)₃. In some embodiments, R^{4b} has one of the following structures:

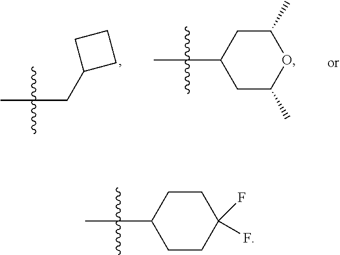

In some embodiments, R^{4b} has the following structure:

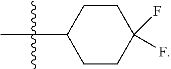 [SMILES: *C1CCC(F)(F)CC1]

In certain embodiments, ring A′ has one of the following structures:

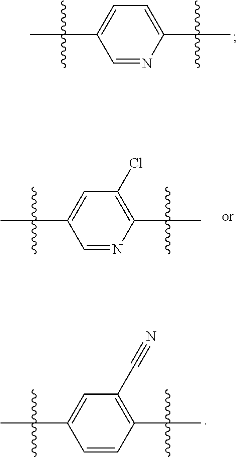 [SMILES: *c1ccc(*)nc1 ; *c1cnc(*)c(Cl)c1 ; *c1ccc(*)c(C#N)c1]

In some embodiments, ring A′ is cyclopropyl. In certain embodiments, ring A′ is cyclobutyl. In some embodiments, ring A′ is cyclopentyl. In certain embodiments, ring A′ is cyclohexyl. In some embodiments, ring A′ is cycloheptyl or cyclooctyl.

In certain embodiments, ring A′ is cyclobutyl, cyclopentyl, or cyclohexyl. In some embodiments, ring A′ is cyclobutyl or cyclohexyl.

In some embodiments, the compound has one of the following Structures (IIB) or (IIC):

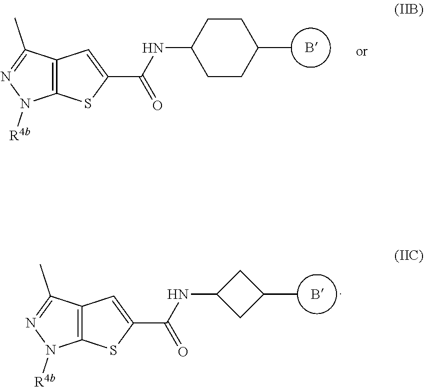

In certain embodiments, ring B′ is optionally substituted with one or more substituents selected from the group consisting of C₁-C₆ alkyl, halo, C₁-C₆ haloalkyl, cyano, C₁-C₃ alkoxy, C₁-C₃ haloalkoxy, —C(═O)alkyl, C(═S)alkyl, C₃-C₆ cycloalkyl, and C(═O)NR^{b1}R^{b2}, wherein:
R^{b1} and R^{b2} are each independently hydrogen or C₁-C₃ alkyl.

In certain embodiments, ring B′ is optionally substituted with one or more substituents selected from the group consisting of C₁-C₃ alkyl (e.g., methyl, ethyl, n-propyl, or isopropyl), halo (e.g., fluoro, chloro, or bromo), C₁-C₃ haloalkyl (e.g., trifluoromethyl, difluoromethyl, trifluoroethyl, difluoroethyl, etc.), cyano, C₁-C₃ alkoxy (e.g., methoxy, ethoxy, n-propoxy, iso-propoxy), C₁-C₃ haloalkoxy, —C(═O)alkyl (e.g., C₁-C₆ alkyl), C(═S)alkyl (e.g., C₁-C₆ alkyl), C₃-C₆ cycloalkyl (e.g., cyclopropyl, cyclobutyl, cyclopentyl, cyclohexyl), and C(═O)NR^{b1}R^{b2}, wherein:
R^{b1} and R^{b2} are each independently hydrogen or C₁-C₃ alkyl (e.g., methyl, ethyl, n-propyl, or isopropyl).

In some embodiments, ring B′ is optionally substituted with one or more substituents selected from the group consisting of —CH(CH₃)₂, —C(═O)CH₃, fluoro, cyclopropyl, —CH₂CF₃, —CH₂CHF₂, C(═O)N(CH₃)₂, —C(═S)CH₃, cyano, —OCHF₂, and —OCH₃.

In some embodiments, ring B′ is azetidinyl, pyrrolidinyl, piperidinyl, morpholinyl, or piperazinyl.

In certain embodiments, ring B′ has one of the following structures:

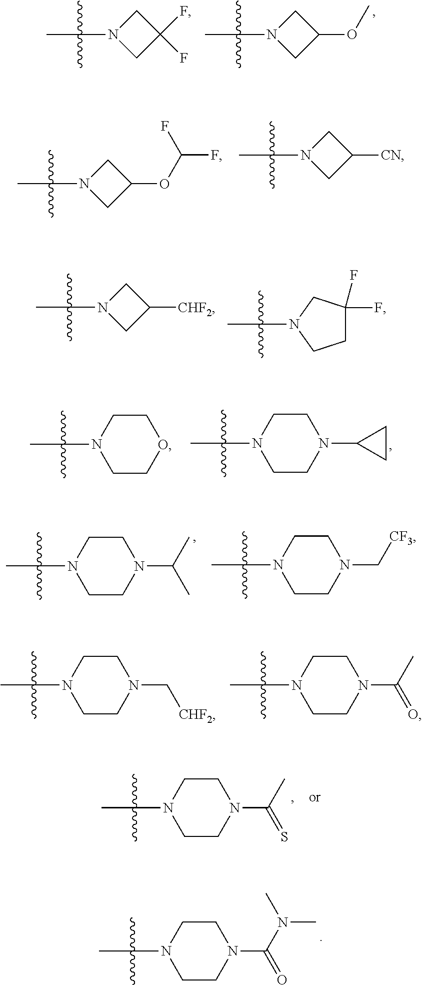

In some embodiments, ring B′ has one of the following structures:

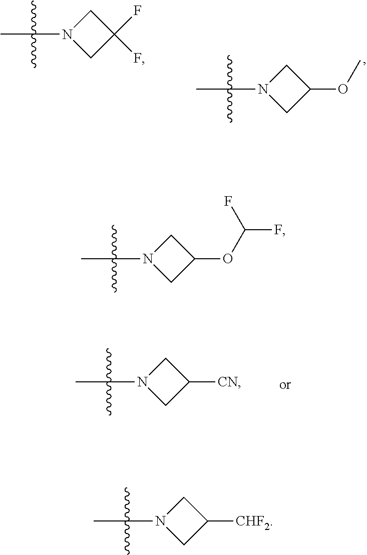

In certain embodiments, ring B′ has one of the following structures:

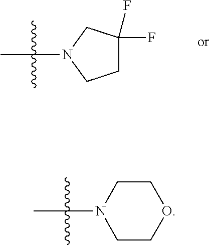 [SMILES: *N1CCC(F)(F)C1 ; *N1CCOCC1]

In some embodiments, ring B′ has one of the following structures:

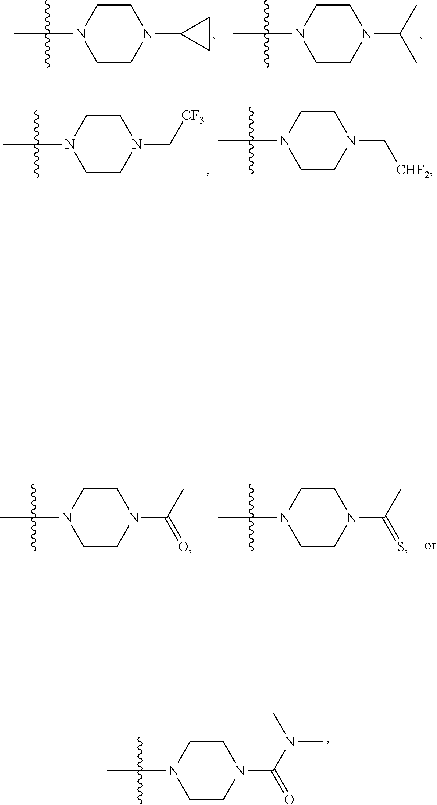

In some embodiments, ring A′ is cyclobutyl, cyclopentyl, or cyclohexyl and ring B′ is azetidinyl, pyrrolidinyl, piperidinyl, morpholinyl, or piperazinyl. In some embodiments, ring A′ is cyclobutyl and ring B′ is azetidinyl, pyrrolidinyl, piperidinyl, morpholinyl, or piperazinyl. In some embodiments, ring A′ is cyclohexyl and ring B′ is azetidinyl, pyrrolidinyl, piperidinyl, morpholinyl, or piperazinyl. In some embodiments, ring A′ is cyclobutyl and ring B′ is azetidinyl, pyrrolidinyl, morpholinyl, or piperazinyl. In certain embodiments, ring A′ is cyclohexyl and ring B′ is azetidinyl, pyrrolidinyl, morpholinyl, or piperazinyl.

In certain embodiments,

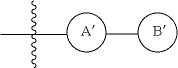

has one of the following structures:

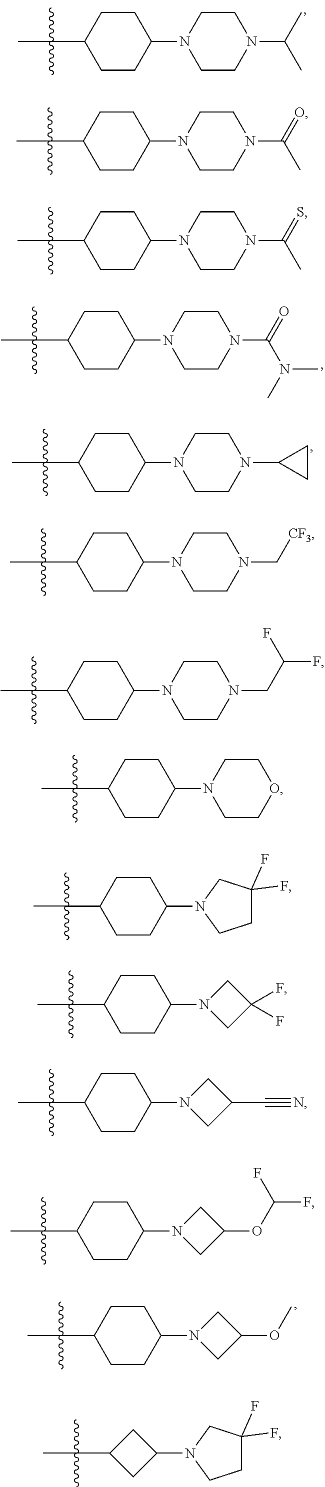

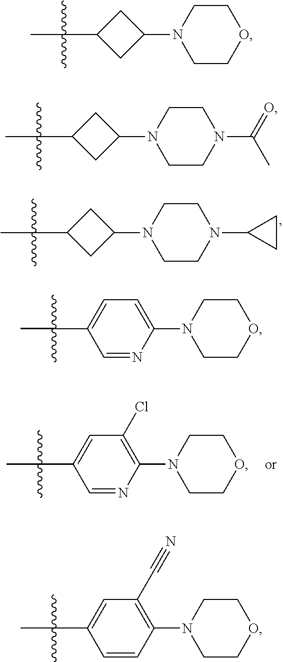

In some embodiments,

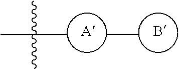

has one of the following structures:

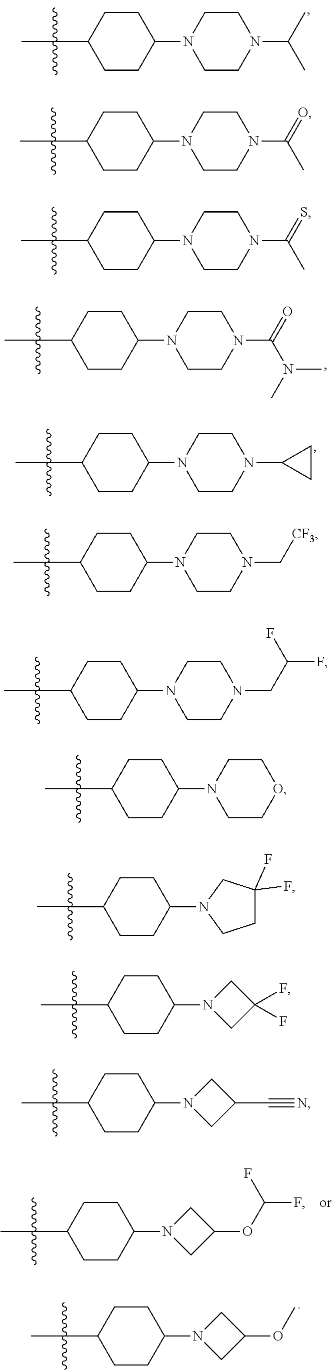

In certain embodiments,

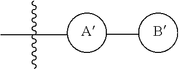

has one of the following structures:

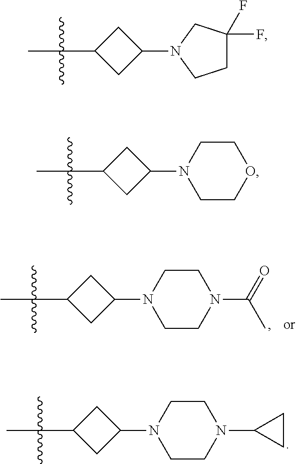 [SMILES: *C1CC(N2CCC(F)(F)C2)C1 ; *C1CC(N2CCOCC2)C1 ; *C1CC(N2CCN(C(C)=O)CC2)C1 ; *C1CC(N2CCN(C3CC3)CC2)C1]

In some embodiments,

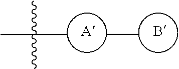

has one of the following structures:

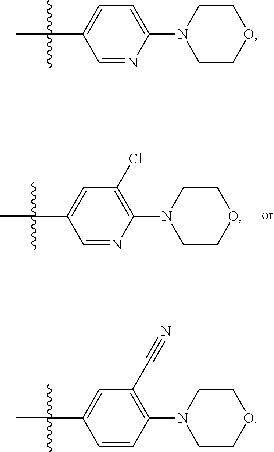 [SMILES: *c1ccc(N2CCOCC2)nc1 ; *c1cnc(N2CCOCC2)c(Cl)c1 ; "or" ; *c1ccc(N2CCOCC2)c(C#N)c1]

In some embodiments, the compound is a free base form. In certain embodiments, the compound is a pharmaceutically acceptable salt. In some embodiments, the compound is a trifluoroacetic acid salt. In some embodiments, the compound is a hydrochloric acid salt. In some embodiments, the compound is a formic acid salt. In certain embodiments, the compound is a tautomer.

In various embodiments, the compound has one of the structures set forth in Table 1 below, as a stereoisomer, enantiomer, or tautomer thereof or a mixture thereof; or a pharmaceutically acceptable salt, solvate, or prodrug thereof. Compounds in Table 1 were prepared as described in the Examples or methods known in the art and analyzed by mass spectrometry and/or ¹H NMR spectroscopy.

TABLE 2
Representative Compounds of Structure (II)

| No. | Structure | Name | Mass^{†} |
|---|---|---|---|
| II-1 | 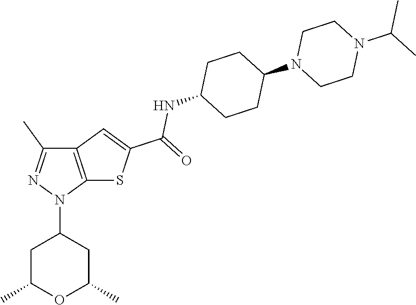 [SMILES: Cc1nn(C2C[C@@H](C)O[C@@H](C)C2)c2sc(C(=O)N[C@H]3CC[C@H](N4CCN(C(C)C)CC4)CC3)cc12] | 1-((2r,6s)-2,6-dimeth- yltetrahydro-2H-pyr- an-4-yl)-N-((1r,4r)-4- (4-isopropylpiperazin- 1-yl)cyclohexyl)-3- methyl-1H-thieno [2,3-c]pyrazole-5- carboxamide | 502.25 |
| II-2 | 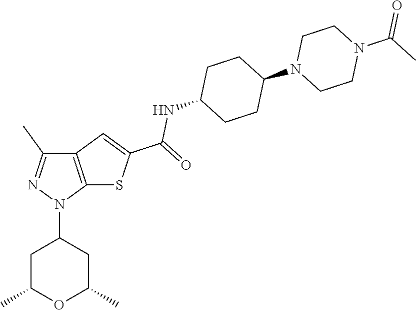 [SMILES: CC(=O)N1CCN([C@H]2CC[C@H](NC(=O)c3cc4c(C)nn(C5C[C@@H](C)O[C@@H](C)C5)c4s3)CC2)CC1] | N-((1r,4r)-4-(4- acetylpiperazin-1- yl)-cyclohexyl)-1- ((2r,6s)-2,6-dimeth- yltetrahydro-2H-pyr- ran-4-yl)-3-methyl- 1H-thieno[2,3-c]pyr- azole-5-carboxamide | 502.21 |
| II-3 | 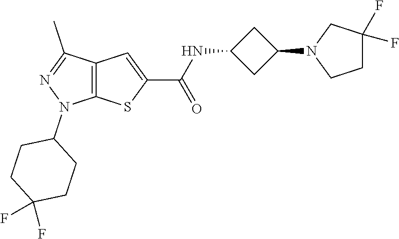 [SMILES: Cc1nn(C2CCC(F)(F)CC2)c2sc(C(=O)N[C@H]3C[C@H](N4CCC(F)(F)C4)C3)cc12] | 1-(4,4-difluorocyclo- hexyl)-N-(3-(3,3- difluoropyrrolidin-1- yl)cyclobutyl)-3- methyl-1H-thieno [2,3-c]pyrazole-5- carboxamide | 459.65 |
| II-4 | 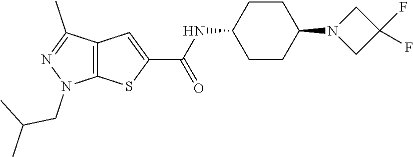 [SMILES: Cc1nn(CC(C)C)c2sc(C(=O)N[C@H]3CC[C@H](N4CC(F)(F)C4)CC3)cc12] | N-((1r,4r)-4-(3,3- difluoroazetidin-1-yl)- cyclohexyl)-1-isobut- yl-3-methyl-1H-thieno [2,3-c]pyrazole-5- carboxamide | 411.15 |
| II-5 | 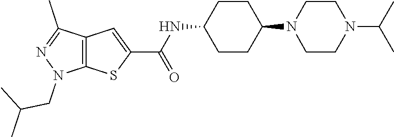 [SMILES: Cc1nn(CC(C)C)c2sc(C(=O)N[C@H]3CC[C@H](N4CCN(C(C)C)CC4)CC3)cc12] | 1-isobutyl-N-((1r,4r)- 4-(4-isopropylpiper- azin-1-yl)cyclohexyl)- 3-methyl-1H-thieno [2,3-c]pyrazole-5- carboxamide | 446.66 |
| II-6 | 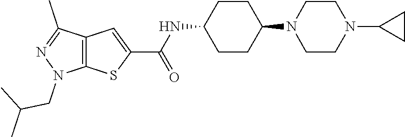 [SMILES: Cc1nn(CC(C)C)c2sc(C(=O)N[C@H]3CC[C@H](N4CCN(C5CC5)CC4)CC3)cc12] | N-((1r,4r)-4-(4-cyclo- propylpiperazin-1-yl)- cyclohexyl)-1-isobut- yl-3-methyl-1H- thieno[2,3-c]pyrazole- 5-carboxamide | 444.76 |
| II-7 | 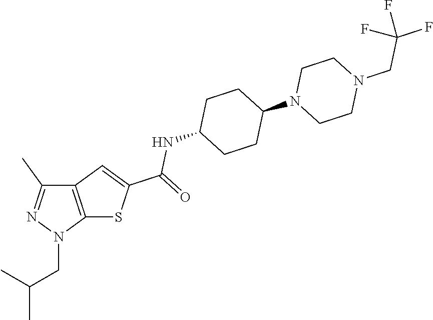 [SMILES: Cc1nn(CC(C)C)c2sc(C(=O)N[C@H]3CC[C@H](N4CCN(CC(F)(F)F)CC4)CC3)cc12] | 1-isobutyl-3-methyl- N-((1r,4r)-4-(4-(2,2,2- trifluoroethyl)piper- azin-1-yl)cyclohexyl)- 1H-thieno[2,3-c]pyr- azole-5-carboxamide | 486.74 |
| II-8 | 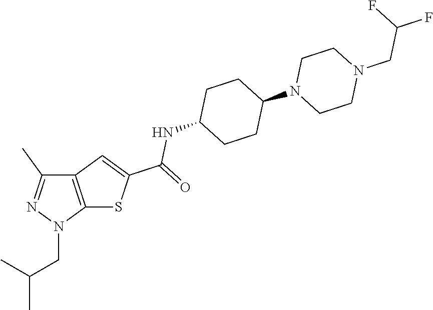 [SMILES: Cc1nn(CC(C)C)c2sc(C(=O)N[C@H]3CC[C@H](N4CCN(CC(F)F)CC4)CC3)cc12] | N-((1r,4r)-4-(4-(2,2- difluoroethyl)piper- azin-1-yl)cyclohexyl)- 1-isobutyl-3-methyl- 1H-thieno[2,3-c]pyr- azole-5-carboxamide | 468.65 |
| II-9 | 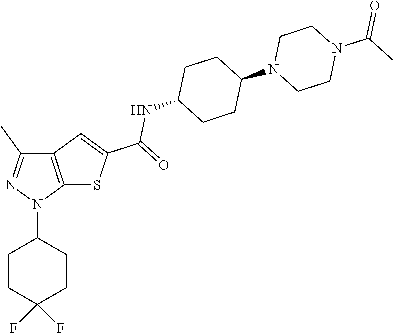 [SMILES: CC(=O)N1CCN([C@H]2CC[C@H](NC(=O)c3cc4c(C)nn(C5CCC(F)(F)CC5)c4s3)CC2)CC1] | N-((1r,4r)-4-(4-acet- ylpiperazin-1-yl)- cyclohexyl)-1-(4,4- difluorocyclohexyl)- 3-methyl-1H-thieno [2,3-c]pyrazole-5- carboxamide | 508.73 |
| II-10 | 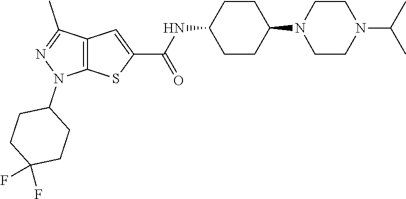 [SMILES: Cc1nn(C2CCC(F)(F)CC2)c2sc(C(=O)N[C@H]3CC[C@H](N4CCN(C(C)C)CC4)CC3)cc12] | 1-(4,4-difluorocyclo- hexyl)-N-((1r,4r)-4- (4-isopropylpiper- azin-1-yl)cyclohex- yl)-3-methyl-1H- thieno[2,3-c]pyra- zole-5-carboxamide | 508.83 |
| II-11 | 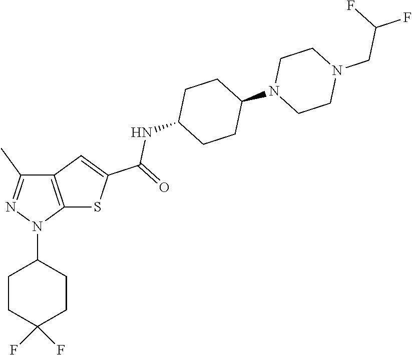 [SMILES: Cc1nn(C2CCC(F)(F)CC2)c2sc(C(=O)N[C@H]3CC[C@H](N4CCN(CC(F)F)CC4)CC3)cc12] | 1-(4,4-difluorocyclo- hexyl)-N-((1r,4r)-4- (4-(2,2-difluoroeth- yl)piperazin-1-yl)- cyclohexyl)-3-meth- yl-1H-thieno[2,3-c] pyrazole-5-carbox- amide | 530.72 |
| II-12 | 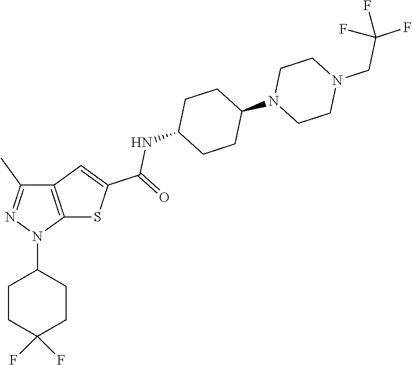 [SMILES: Cc1nn(C2CCC(F)(F)CC2)c2sc(C(=O)N[C@H]3CC[C@H](N4CCN(CC(F)(F)F)CC4)CC3)cc12] | 1-(4,4-difluorocyclo- hexyl)-N-((1r,4r)-4- (4-(2,2-trifluoroeth- yl)piperazin-1-yl)- cyclohexyl)-3-meth- yl-1H-thieno[2,3-c] pyrazole-5-carbox- amide | 548.61 |
| II-13 | 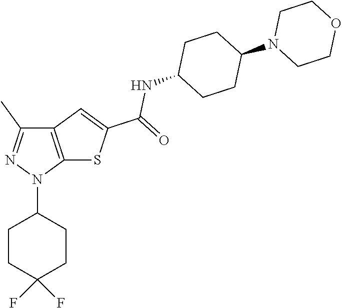 [SMILES: Cc1nn(C2CCC(F)(F)CC2)c2sc(C(=O)N[C@H]3CC[C@H](N4CCOCC4)CC3)cc12] | 1-(4,4-difluorocyclo- hexyl)-3-methyl-N- ((1r,4r)-4-morpho- linocyclohexyl)-1H- thieno[2,3-c]pyrazole- 5-carboxamide | 467.65 |
| II-14 | 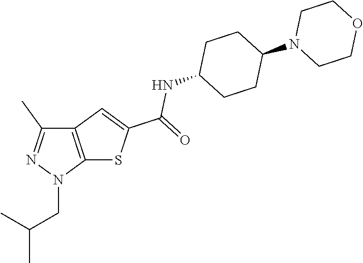 [SMILES: Cc1nn(CC(C)C)c2sc(C(=O)N[C@H]3CC[C@H](N4CCOCC4)CC3)cc12] | 1-(4,4-difluorocyclo- hexyl)-3-methyl-N- ((1r,4r)-4-morpho- lino-cyclohexyl)-1H- thieno[2,3-c]pyrazole- 5-carboxamide | 405.68 |
| II-15 | 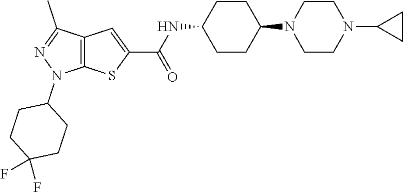 [SMILES: Cc1nn(C2CCC(F)(F)CC2)c2sc(C(=O)N[C@H]3CC[C@H](N4CCN(C5CC5)CC4)CC3)cc12] | N-((1r,4r)-4-(4-cyclo- propylpiperazin-1- yl)cyclohexyl)-1-(4,4- difluorocyclohexyl)- 3-methyl-1H-thieno [2,3-c]pyrazole-5- carboxamide | 506.73 |
| II-16 | 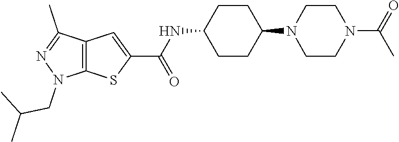 [SMILES: CC(=O)N1CCN([C@H]2CC[C@H](NC(=O)c3cc4c(C)nn(CC(C)C)c4s3)CC2)CC1] | N-((1r,4r)-4-(4-acet- ylpiperazin-1-yl)- cyclohexyl)-1-iso- butyl-3-methyl-1H thieno[2,3-c]pyrazole- 5-carboxamide | 446.76 |
| II-17 | 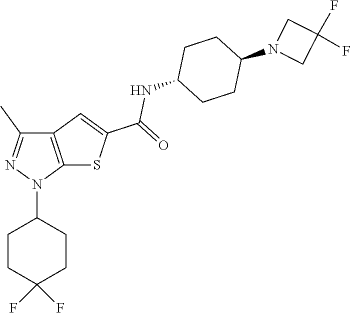 [SMILES: Cc1nn(C2CCC(F)(F)CC2)c2sc(C(=O)N[C@H]3CC[C@H](N4CC(F)(F)C4)CC3)cc12] | N-((1r,4r)-4-(3,3- difluoroazetidin-1- yl)cyclohexyl)-1-(4,4- difluorocyclohexyl)- 3-methyl-1H-thieno [2,3-c]pyrazole-5- carboxamide | 473.65 |
| II-18 | 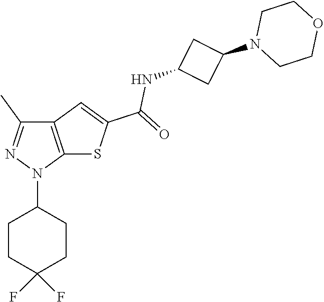 [SMILES: Cc1nn(C2CCC(F)(F)CC2)c2sc(C(=O)N[C@H]3C[C@H](N4CCOCC4)C3)cc12] | 1-(4,4-difluorocyclo- hexyl)-3-methyl-N- ((1r,3r)-3-morpho- linocyclobutyl)-1H- thieno[2,3-c]pyrazole- 5-carboxamide | 439.66 |
| II-19 | 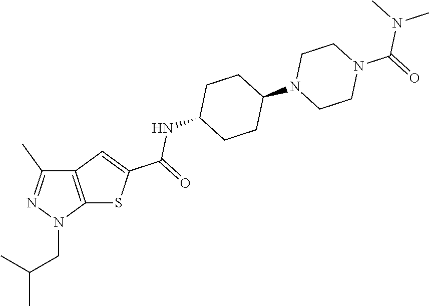 [SMILES: Cc1nn(CC(C)C)c2sc(C(=O)N[C@H]3CC[C@H](N4CCN(C(=O)N(C)C)CC4)CC3)cc12] | N-((1r,4r)-4-(4- (dimethylcarbamo- yl)piperazin-1-yl)- cyclohexyl)-1-iso- butyl-3-methyl-1H- thieno[2,3-c]pyrazole- 5-carboxamide | 475.75 |
| II-20 | 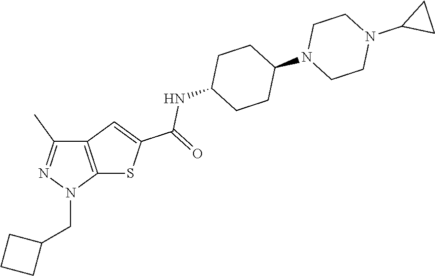 [SMILES: Cc1nn(CC2CCC2)c2sc(C(=O)N[C@H]3CC[C@H](N4CCN(C5CC5)CC4)CC3)cc12] | 1-(cyclobutylmethyl)- N-((1r,4r)-4-(4- cyclopropylpiperazin- 1-yl)cyclohexyl)-3- methyl-1H-thieno [2,3-c]pyrazole-5- carboxamide | 456.75 |
| II-21 | 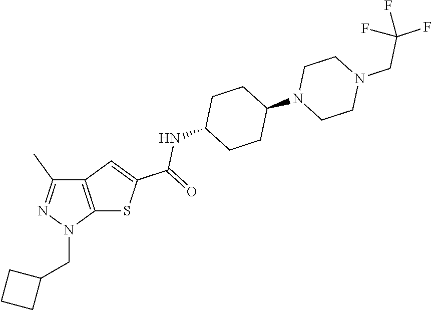 [SMILES: Cc1nn(CC2CCC2)c2sc(C(=O)N[C@H]3CC[C@H](N4CCN(CC(F)(F)F)CC4)CC3)cc12] | 1-(cyclobutylmethyl)- 3-methyl-N-((1r,4r)- 4-(4-(2,2,2-trifluoro- ethyl)piperazin-1-yl)- cyclohexyl)-1H- thieno[2,3-c]pyrazole- 5-carboxamide | 498.73 |
| II-22 | 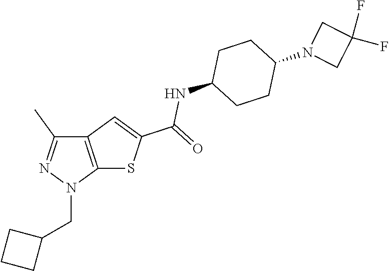 [SMILES: Cc1nn(CC2CCC2)c2sc(C(=O)N[C@H]3CC[C@H](N4CC(F)(F)C4)CC3)cc12] | 1-(cyclobutylmethyl)- N-((1r,4r)-4-(3,3- difluoroazetidin-1- yl)cyclohexyl)-3- methyl-1H-thieno [2,3-c]pyrazole-5- carboxamide | 423.67 |
| II-23 | 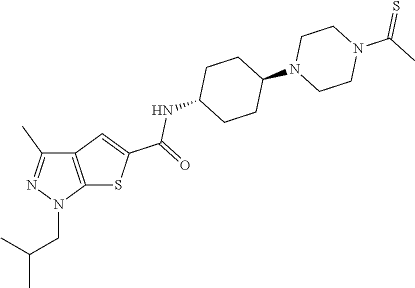 [SMILES: CC(=S)N1CCN([C@H]2CC[C@H](NC(=O)c3cc4c(C)nn(CC(C)C)c4s3)CC2)CC1] | N-((1r,4r)-4-(4- ethanethioylpiperazin- 1-yl)cyclohexyl)-1- isobutyl-3-methyl-1H- thieno[2,3-c]pyrazole- 5-carboxamide | 462.20 |
| II-24 | 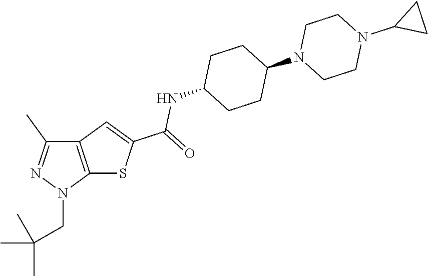 [SMILES: Cc1nn(CC(C)(C)C)c2sc(C(=O)N[C@H]3CC[C@H](N4CCN(C5CC5)CC4)CC3)cc12] | N-((1r,4r)-4-(4-cyclo- propylpiperazin-1-yl)- cyclohexyl)-3-meth- yl-1-neopentyl-1H- thieno[2,3-c]pyrazole- 5-carboxamide | 458.75 |
| II-25 | 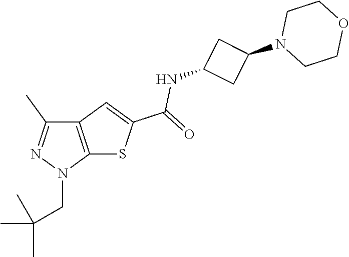 [SMILES: Cc1nn(CC(C)(C)C)c2sc(C(=O)N[C@H]3C[C@H](N4CCOCC4)C3)cc12] | 3-methyl-N-((1r,3r)- 3-morpholinocyclo- butyl)-1-neopentyl- 1H-thieno[2,3-c]pyr- azole-5-carboxamide | 391.68 |
| II-26 | 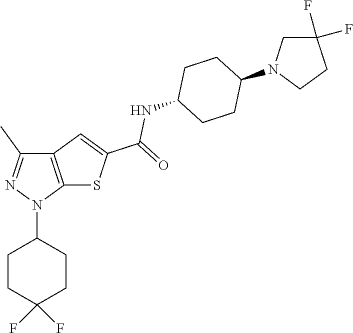 [SMILES: Cc1nn(C2CCC(F)(F)CC2)c2sc(C(=O)N[C@H]3CC[C@H](N4CCC(F)(F)C4)CC3)cc12] | 1-(4,4-difluorocyclo- hexyl)-N-((1r,4r)-4- (3,3-difluoropyrrol- idin-1-yl)cyclohexyl)- 3-methyl-1H-thieno [2,3-c]pyrazole-5- carboxamide | 487.64 |
| II-27 | 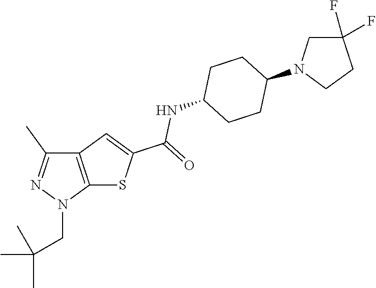 [SMILES: Cc1nn(CC(C)(C)C)c2sc(C(=O)N[C@H]3CC[C@H](N4CCC(F)(F)C4)CC3)cc12] | N-((1r,4r)-4-(3,3- difluoropyrrolidin-1- yl)cyclohexyl)-3- methyl-1-neopentyl- 1H-thieno[2,3-c]pyr- azole-5-carboxamide | 439.66 |
| II-28 | 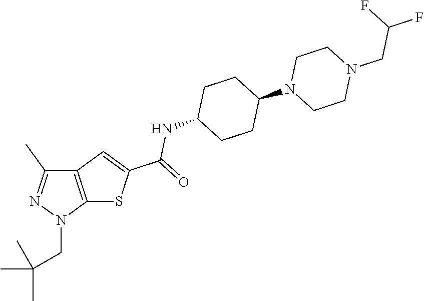 [SMILES: Cc1nn(CC(C)(C)C)c2sc(C(=O)N[C@H]3CC[C@H](N4CCN(CC(F)F)CC4)CC3)cc12] | N-((1r,4r)-4-(4-(2,2- difluoroethyl)piper- azin-1-yl)cyclohex- yl)-3-methyl-1-neo- pentyl-1H-thieno [2,3-c]pyrazole-5- carboxamide | 482.74 |
| II-29 | 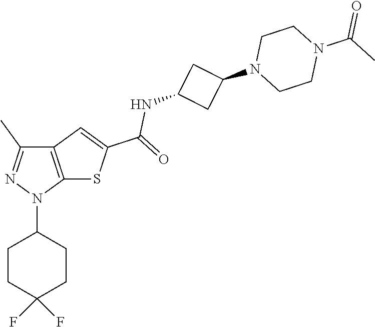 [SMILES: CC(=O)N1CCN([C@H]2C[C@H](NC(=O)c3cc4c(C)nn(C5CCC(F)(F)CC5)c4s3)C2)CC1] | N-((1r,3r)-3-(4-acet- ylpiperazin-1-yl)- cyclobutyl)-1-(4,4- difluorocyclohexyl)- 3-methyl-1H-thieno [2,3-c]pyrazole-5- carboxamide | 480.64 |
| II-30 | 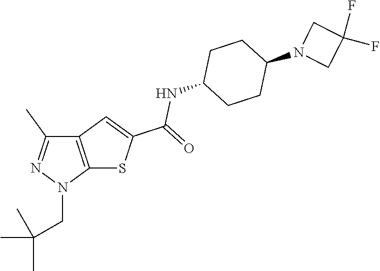 [SMILES: Cc1nn(CC(C)(C)C)c2sc(C(=O)N[C@H]3CC[C@H](N4CC(F)(F)C4)CC3)cc12] | N-((1r,4r)-4-(3,3- difluoroazetidin-1- yl)cyclohexyl)-3- methyl-1-neopentyl- 1H-thieno[2,3-c]pyr- azole-5-carboxamide | 425.67 |
| II-31 | 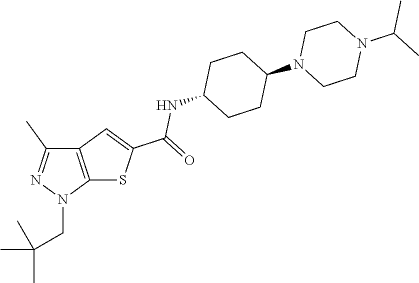 [SMILES: Cc1nn(CC(C)(C)C)c2sc(C(=O)N[C@H]3CC[C@H](N4CCN(C(C)C)CC4)CC3)cc12] | N-((1r,4r)-4-(4-iso- propylpiperazin-1- yl)cyclohexyl)-3- methyl-1-neopentyl- 1H-thieno[2,3-c]pyr- azole-5-carboxamide | 460.75 |
| II-32 | 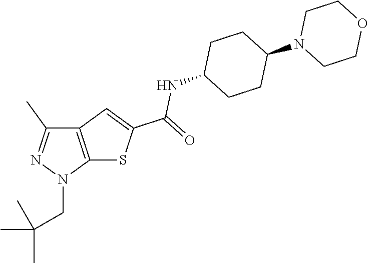 [SMILES: Cc1nn(CC(C)(C)C)c2sc(C(=O)N[C@H]3CC[C@H](N4CCOCC4)CC3)cc12] | 3-methyl-N-((1r,4r)- 4-morpholinocyclo- hexyl)-1-neopentyl- 1H-thieno[2,3-c]pyr- azole-5-carboxamide | 419.67 |
| II-33^{‡} | 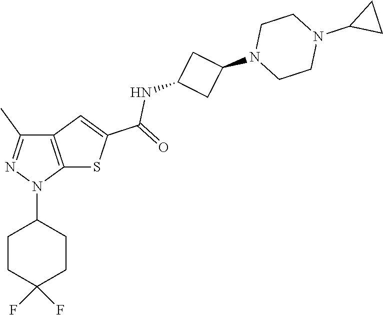 [SMILES: Cc1nn(C2CCC(F)(F)CC2)c2sc(C(=O)N[C@H]3C[C@H](N4CCN(C5CC5)CC4)C3)cc12] | N-((1r,3r)-3-(4-cyclo- propylpiperazin-1- yl)cyclobutyl)-1-(4,4- difluorocyclohexyl)- 3-methyl-1H-thieno [2,3-c]pyrazole-5- carboxamide | 478.74 |
| II-34^{‡} | 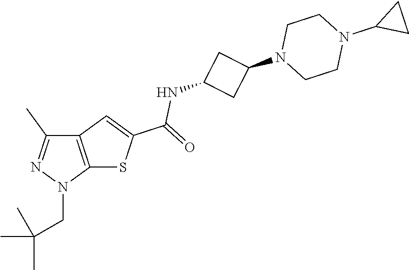 [SMILES: Cc1nn(CC(C)(C)C)c2sc(C(=O)N[C@H]3C[C@H](N4CCN(C5CC5)CC4)C3)cc12] | N-((1r,3r)-3-(4-cyclo- propylpiperazin-1- yl)cyclobutyl)-3- methyl-1-neopentyl- 1H-thieno[2,3-c] pyrazole-5-carbox- amide | 430.77 |
| II-35 | 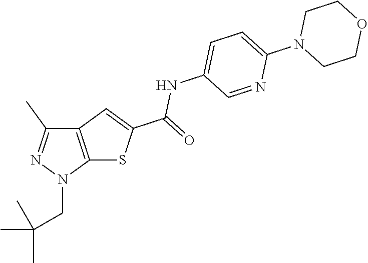 [SMILES: Cc1nn(CC(C)(C)C)c2sc(C(=O)Nc3ccc(N4CCOCC4)nc3)cc12] | 3-methyl-N-(6- morpholinopyridin- 3-yl)-1-neopentyl- 1H-thieno[2,3-c]pyr- azole-5-carboxamide | 414.67 |
| II-36 | 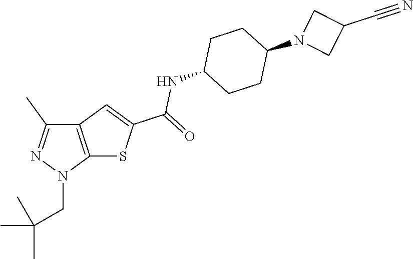 [SMILES: Cc1nn(CC(C)(C)C)c2sc(C(=O)N[C@H]3CC[C@H](N4CC(C#N)C4)CC3)cc12] | N-((1r,4r)-4-(3- cyanoazetidin-1-yl)- cyclohexyl)-3-meth- yl-1-neopentyl-1H- thieno[2,3-c]pyr- azole-5-carboxamide | 414.67 |
| II-37^{‡} | 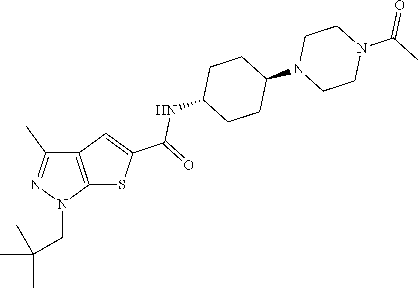 [SMILES: CC(=O)N1CCN([C@H]2CC[C@H](NC(=O)c3cc4c(C)nn(CC(C)(C)C)c4s3)CC2)CC1] | N-((1r,4r)-4-(4-acet- ylpiperazin-1-yl)- cyclohexyl)-3-meth- yl-1-neopentyl-1H- thieno[2,3-c]pyr- azole-5-carboxamide | 460.75 |
| II-38 | 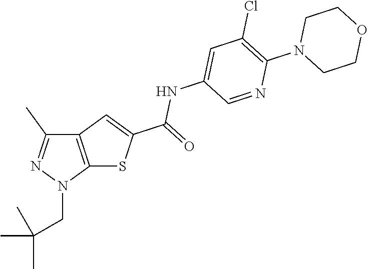 [SMILES: Cc1nn(CC(C)(C)C)c2sc(C(=O)Nc3cnc(N4CCOCC4)c(Cl)c3)cc12] | N-(5-chloro-6- morpholinopyridin- 3-yl)-3-methyl-1- neopentyl-1H-thieno [2,3-c]pyrazole-5- carboxamide | 448.66 |
| II-39 | 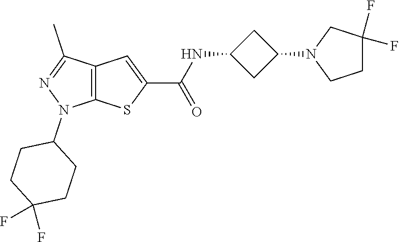 [SMILES: Cc1nn(C2CCC(F)(F)CC2)c2sc(C(=O)N[C@H]3C[C@@H](N4CCC(F)(F)C4)C3)cc12] | 1-(4,4-difluorocyclo- hexyl)-N-((1s,3s)-3- (3,3-difluoropyrrol- idin-1-yl)cyclobutyl)- 3-methyl-1H-thieno [2,3-c]pyrazole-5- carboxamide | 459.65 |
| II-40 | 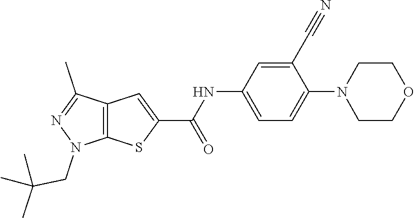 [SMILES: Cc1nn(CC(C)(C)C)c2sc(C(=O)Nc3ccc(N4CCOCC4)c(C#N)c3)cc12] | N-(3-cyano-4-mor- pholinophenyl)-3- methyl-1-neopentyl- 1H-thieno[2,3-c]pyr- azole-5-carboxamide | 438.66 |
| II-41 | 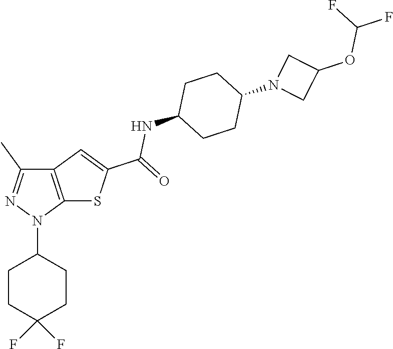 [SMILES: Cc1nn(C2CCC(F)(F)CC2)c2sc(C(=O)N[C@H]3CC[C@H](N4CC(OC(F)F)C4)CC3)cc12] | 1-(4,4-difluorocyclo- hexyl)-N-((1r,4r)-4- (3-(difluorometh- oxy)azetidin-1-yl)- cyclohexyl)-3- methyl-1H-thieno [2,3-c]pyrazole-5- carboxamide | 503.63 |
| II-42 | 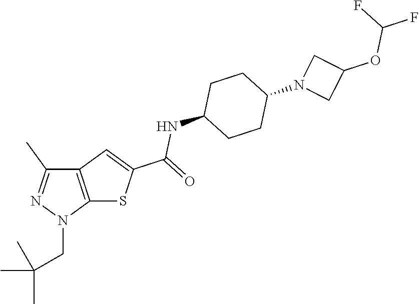 [SMILES: Cc1nn(CC(C)(C)C)c2sc(C(=O)N[C@H]3CC[C@H](N4CC(OC(F)F)C4)CC3)cc12] | N-((1r,4r)-4-(3- (difluoromethoxy)- azetidin-1-yl)cyclo- hexyl)-3-methyl-1- neopentyl-1H-thieno [2,3-c]pyrazole-5- carboxamide | 455.66 |
| II-43 | 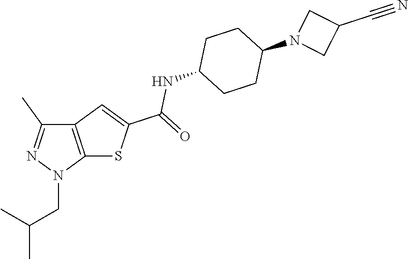 [SMILES: Cc1nn(CC(C)C)c2sc(C(=O)N[C@H]3CC[C@H](N4CC(C#N)C4)CC3)cc12] | N-((1r,4r)-4-(3- cyanoazetidin-1-yl)- cyclohexyl)-1-iso- butyl-3-methyl-1H- thieno[2,3-c]pyrazole- 5-carboxamide | 400.68 |
| II-44 | 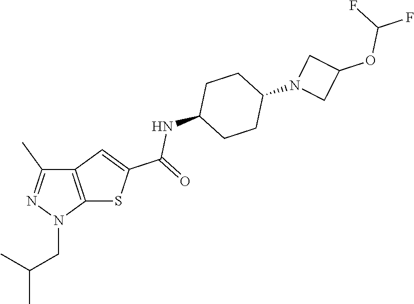 [SMILES: Cc1nn(CC(C)C)c2sc(C(=O)N[C@H]3CC[C@H](N4CC(OC(F)F)C4)CC3)cc12] | N-((1r,4r)-4-(3- (difluoromethoxy)- azetidin-1-yl)cyclo- hexyl)-1-isobutyl-3- methyl-1H-thieno [2,3-c]pyrazole-5- carboxamide | 441.66 |
| II-45 | 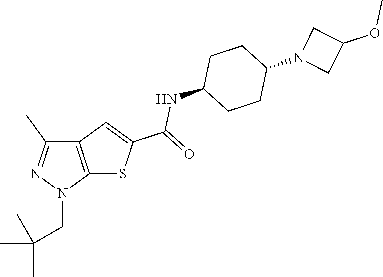 [SMILES: COC1CN([C@H]2CC[C@H](NC(=O)c3cc4c(C)nn(CC(C)(C)C)c4s3)CC2)C1] | N-((1r,4r)-4-(3- methoxyazetidin-1- yl)cyclohexyl)-3- methyl-1-neopentyl- 1H-thieno[2,3-c]pyr- azole-5-carboxamide | 419.67 |
| II-46 | 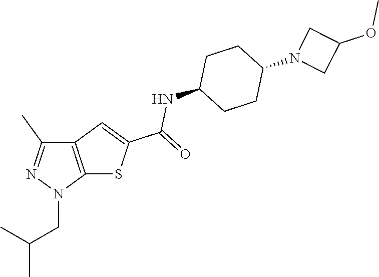 [SMILES: COC1CN([C@H]2CC[C@H](NC(=O)c3cc4c(C)nn(CC(C)C)c4s3)CC2)C1] | 1-isobutyl-N-((1r,4r)- 4-(3-methoxyazeti- din-1-yl)cyclohexyl)- 3-methyl-1H-thieno [2,3-c]pyrazole-5- carboxamide | 405.58 |

^{†}Observed mass: Positive Electrospray [M + H]⁺
^{‡}Also obtained as a trifluoroacetate salt

In some embodiments, the compound has a molecular weight of less than about 500 g/mole. In some embodiments, the compound has a molecular weight of less than about 450 g/mole. In certain embodiments, the compound has an IC₅₀ for inhibiting PDE 7A and/or PDE 7B activity of less than about 1 μM. In some embodiments, the compound has an IC₅₀ for inhibiting PDE 7A and/or PDE 7B activity of less than about 100 nM. In certain embodiments, the compound is a selective PDE 7 inhibitor for which the lesser of the IC₅₀ for inhibiting PDE 7A activity and the IC₅₀ for inhibiting PDE 7B activity is less than one-tenth the IC₅₀ that the compound has for inhibiting the activity of any other PDE enzyme from the PDE 1-6 and PDE 8-11 enzyme families. In certain embodiments, the compound is a highly selective PDE 7 inhibitor for which the lesser of the IC₅₀ for inhibiting PDE 7A activity and the IC₅₀ for inhibiting PDE 7B activity is less than one-fiftieth the IC₅₀ that the compound has for inhibiting the activity of any other PDE enzyme from the PDE 1-6 and PDE 8-11 enzyme families.

It is understood that in the present description, combinations of substituents and/or variables of the depicted formulae are permissible only if such contributions result in stable compounds.

In an additional embodiment, various compounds of the disclosure which exist in free base or acid form can be converted to their pharmaceutically acceptable salts by treatment with the appropriate inorganic or organic base or acid by methods known to one skilled in the art. Salts of the compounds of the disclosure can be converted to their free base or acid form by standard techniques.

Methods for producing the compounds described herein are provided in the Examples that follow. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5^{th} edition (Wiley, December 2000)) or prepared as described herein.

### Pharmaceutical Compositions

Other embodiments are directed to pharmaceutical compositions. The pharmaceutical composition comprises anyone (or more) of the foregoing compounds and a pharmaceutically acceptable carrier. In some embodiments, the pharmaceutical composition is formulated for oral administration. In other embodiments, the pharmaceutical composition is formulated for injection. In still more embodiments, the pharmaceutical compositions comprise a compound as disclosed herein and an additional therapeutic agent (e.g., anticancer agent). Non-limiting examples of such therapeutic agents are described herein below.

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long-acting formulations are administered by implantation (for example subcutaneously or intramuscularly) or by intramuscular injection. Furthermore, in other embodiments, the compound is delivered in a targeted drug delivery system, for example, in a liposome coated with and organ-specific antibody. In such embodiments, the liposomes are targeted to and taken up selectively by the organ. In yet other embodiments, the compound as described herein is provided in the form of a rapid release formulation, in the form of an extended-release formulation, or in the form of an intermediate release formulation. In yet other embodiments, the compound described herein is administered topically.

In treatment methods according to embodiments of the disclosure, an effective amount of at least one compound of Structure (I) or (II) is administered to a subject suffering from or diagnosed as having such a disease, disorder, or medical condition. Effective amounts or doses may be ascertained by methods such as modeling, dose escalation studies or clinical trials, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician.

The compounds according to the disclosure are effective over a wide dosage range. For example, in the treatment of adult humans, dosages from 10 to 5000 mg, from 100 to 5000 mg, from 1000 mg to 4000 mg per day, and from 1000 to 3000 mg per day are examples of dosages that are used in some embodiments. The exact dosage will depend upon the route of administration, the form in which the compound is administered, the subject to be treated, the body weight of the subject to be treated, and the preference and experience of the attending physician.

In some embodiments, compounds of the disclosure are administered in a single dose. Typically, such administration will be by injection, e.g., intravenous injection, in order to introduce the agent quickly. However, other routes are used as appropriate. A single dose of a compound of the disclosure may also be used for treatment of an acute condition.

In some embodiments, compounds of the disclosure are administered in multiple doses. In some embodiments, dosing is about once, twice, three times, four times, five times, six times, or more than six times per day. In other embodiments, dosing is about once a month, once every two weeks, once a week, or once every other day. In another embodiment compounds of the disclosure and another agent (e.g., anti-cancer agent) are administered together about once per day to about 6 times per day. In another embodiment the administration of compounds of the disclosure and an agent continues for less than about 7 days. In yet another embodiment the administration continues for more than about 6, 10, 14, 28 days, two months, six months, or one year. In some cases, continuous dosing is achieved and maintained as long as necessary.

Administration of compounds of the disclosure may continue as long as necessary. In some embodiments, compounds of the disclosure are administered for more than 1, 2, 3, 4, 5, 6, 7, 14, or 28 days. In some embodiments, compounds of the disclosure are administered for less than 28, 14, 7, 6, 5, 4, 3, 2, or 1 day. In some embodiments, compounds of the disclosure are administered chronically on an ongoing basis, e.g., for the treatment of chronic effects.

In some embodiments, the compounds of the disclosure are administered in individual dosage forms. It is known in the art that due to intersubject variability in compound pharmacokinetics, individualization of dosing regimen is necessary for optimal therapy.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the disclosed compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more compounds of Structure (I) or (II), and a pharmaceutically acceptable carrier.

Provided herein are pharmaceutical compositions comprising one or more compounds selected from compounds of Structure (I) or (II) and pharmaceutically acceptable diluent(s), excipient(s), and carrier(s). In certain embodiments, the compounds described are administered as pharmaceutical compositions in which one or more compounds selected from compounds of Structure (I) or (II) are mixed with other active ingredients, as in combination therapy. Encompassed herein are all combinations of actives set forth in the combination therapies section below and throughout this disclosure. In specific embodiments, the pharmaceutical compositions include one or more compounds of Structure (I) or (II).

In a specific embodiment, pharmaceutical compositions of the compounds of Structure (I) or (II) inhibit PDE 7 activity when administered to a patient or a biological sample.

A pharmaceutical composition, as used herein, refers to a mixture of one or more compounds selected from compounds of Structure (I) or (II) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, therapeutically effective amounts of one or more compounds selected from compounds of Structure (I) or (II) provided herein are administered in a pharmaceutical composition to a mammal having a disease, disorder, or medical condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, one or more compounds selected from compounds of Structure (I) or (II) are formulated in aqueous solutions. In specific embodiments, the aqueous solution is selected from, by way of example only, a physiologically compatible buffer, such as Hank's solution, Ringer's solution, or physiological saline buffer. In other embodiments, one or more compounds selected from compounds of Structure (I) or (II) are formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or non-aqueous solutions. In specific embodiments, such solutions include physiologically compatible buffers and/or excipients.

In another embodiment, compounds described herein are formulated for oral administration. Compounds described herein are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions, and the like.

In certain embodiments, pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

One embodiment provides a method of administering a therapeutically effective amount of a PDE 7 inhibitor to a subject in need thereof, wherein the administering is oral administration.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

Another embodiment provides a method of administering a therapeutically effective amount of a PDE 7 inhibitor to a subject in need thereof, wherein the administering is via an injection (e.g., intravenous, intramuscular, subcutaneous, intraosseous, or intradermal injection).

In still other embodiments, the compounds described herein are formulated for parenteral injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions are formulated in a form suitable for parenteral injection as sterile suspensions, solutions, or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of one or more compounds selected from compounds of Structure (I) or (II) are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

One embodiment provides a PDE 7 inhibitor as part of a pharmaceutical composition that further comprises a pharmaceutically acceptable carrier, diluent, excipient, etc. or combinations thereof. Pharmaceutical compositions include at least one pharmaceutically acceptable carrier, diluent, or excipient, and one or more compounds selected from compounds of Structure (I) or (II), described herein as an active ingredient. The active ingredient is in free-acid or free-base form, or in a pharmaceutically acceptable salt form. In addition, the methods and pharmaceutical compositions described herein include the use of N-oxides, crystalline forms (also known as polymorphs), solvates, isotopologues, as well as active metabolites of these compounds having the same type of activity. All tautomers of the compounds described herein are included within the scope of the compounds presented herein. Additionally, the compounds described herein encompass un-solvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting, or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions, and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical compositions comprising one or more compounds selected from compounds of Structure (I) or (II) illustratively takes the form of a liquid where the agents are present in solution, in suspension or both. Typically, when the composition is administered as a suspension, a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, aqueous suspensions contain one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally, include solubilizing agents to aid in the solubility of one or more compounds selected from compounds of Structure (I) or (II). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric, and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Compositions may include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Compositions may include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers useful herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few weeks up to over 100 days. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, the concentration of one or more compounds selected from compounds of Structure (I) or (II) provided in the pharmaceutical compositions of the present disclosure is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more compounds selected from compounds of Structure (I) or (II) provided in the pharmaceutical compositions of the present disclosure is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the amount the one or more compounds selected from compounds of Structure (I) or (II) provided in the pharmaceutical compositions of the present disclosure is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount the one or more compounds selected from compounds of Structure (I) or (II) provided in the pharmaceutical compositions of the present disclosure is equal to or greater than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the one or more compounds selected from compounds of Structure (I) or (II) provided in the pharmaceutical compositions of the present disclosure is in the range of 0.0001-10 g, 0.0005-9 g, 0.001-8 g, 0.005-7 g, 0.01-6 g, 0.05-5 g, 0.1-4 g, 0.5-4 g, or 1-3 g.

In some embodiments, the amount of the one or more compounds selected from compounds of Structure (I) or (II) provided in the pharmaceutical compositions of the present disclosure is in the range of 0.0001-3 g, 0.0005-3 g, 0.001-3 g, 0.005-3 g, 0.01-3 g, 0.05-3 g, 0.1-3 g, or 0.5-3 g.

In some embodiments, the amount of the one or more compounds selected from compounds of Structure (I) or (II) provided in the pharmaceutical compositions of the present disclosure is in the range of 0.0001-10 g, 0.0001-9 g, 0.0001-8 g, 0.0001-7 g, 0.0001-6 g, 0.0001-5 g, 0.0001-4 g, 0.0001-4 g, or 0.0001-3 g.

Packaging materials for use in packaging pharmaceutical compositions described herein include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more compounds described herein, optionally in a composition or in combination with another agent as disclosed herein. The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded, or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical compositions are presented in a pack or dispenser device which contains one or more unit dosage forms containing a compound provided herein. The pack for example contains metal or plastic foil, such as a blister pack. Or the pack or dispenser device is accompanied by instructions for administration. Or the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions containing a compound provided herein formulated in a compatible pharmaceutical carrier are prepared, placed in an appropriate container, and labeled for treatment of an indicated condition.

### Methods of Treatment

The compounds of the present disclosure are useful for treating conditions, diseases, and disorders that are modulated at least in part by phosphodiesterase 7 (PDE 7). Accordingly, one embodiment provides a use of a compound of Structure (I) for the manufacture of a medicament for treatment of a condition, disease, or disorder modulated in part by PDE 7. Another embodiment provides a use of a compound of Structure (II) for the manufacture of a medicament for treatment of a condition, disease, or disorder modulated in part by PDE 7.

One embodiment provides a method of specifically hydrolyzing cyclic 3′, 5′-adenosine monophosphate (cAMP) comprising administering a PDE 7 inhibitor (e.g., a compound of Structure (I) or Structure (II)) to a subject in need thereof. Another embodiment provides a method for regulating intracellular cAMP signaling comprising administering a PDE 7 inhibitor (e.g., a compound of Structure (I) or Structure (II)) to a subject in need thereof.

In some more specific embodiments, the condition, disease, or disorder is addiction. Still another embodiment provides a method of treating an addiction to an addictive agent, comprising administering to a subject in need thereof an amount of an inhibitor of a phosphodiesterase 7 (PDE 7) of a compound of Structure (I) or (II) effective for the treatment of the addiction, as a stereoisomer, enantiomer, or tautomer thereof or a mixture thereof; or a pharmaceutically acceptable salt, solvate, or prodrug thereof.

In some embodiments, the subject is addicted to an addictive agent selected from the group consisting of alcohol, nicotine, marijuana, marijuana derivatives, opioid agonists, benzodiazepines, barbiturates, and psychostimulants. In some embodiments, the addictive agent is alcohol. In certain embodiments, the addictive agent is nicotine. In some embodiments, the opioid agonist is selected from the group consisting of morphine, methadone, fentanyl, sufentanil, and heroin. In certain embodiments, the psychostimulant is cocaine, amphetamines, or amphetamine derivatives. In some embodiments, the psychostimulant is cocaine.

In some embodiments, the condition, disease, or disorder is a movement disorder (e.g., a neurological condition that causes problems with movement). In some embodiments, the movement disorder is a tremor, Tourette syndrome, dystonia, Parkinson's disease, Huntington's disease, multiple system atrophy (MSA), myoclonus, progressive supranuclear palsy, Rett syndrome, secondary parkinsonism, spasticity, or Wilson's disease.

In some embodiments, the condition, disease, or disorder is selected from the group consisting of allergic diseases, inflammatory diseases, or immunological diseases. In some embodiments, a condition, disease, or disorder is bronchial asthma, chronic bronchitis, chronic obstructive pulmonary disease, allergic rhinitis, psoriasis, atopic dermatitis, conjunctivitis, osteoarthritis, rheumatoid arthritis, multiple sclerosis, systemic lupus erythematosus, inflammatory bowel disease, hepatitis, pancreatitis, encephalomyelitis, sepsis, Crohn's disease, rejection reaction in transplantation, graft versus host disease (GVH disease), and restenosis after angioplasty.

In some embodiments, the condition, disease, or disorder is a primary impulse control disorder is a food addiction, binge eating, pathological gambling, pathological use of electronic devices, pathological use of electronic video games, pathological use of electronic communication devices, pathological use of cellular telephones, addiction to pornography, sex addiction, compulsive spending, anorexia, bulimia, intermittent explosive disorder, kleptomania, pyromania, trichotillomania, compulsive over-exercising, and compulsive overworking. In another embodiment, condition, disease, or disorder is an addictive or compulsive behavior. In some embodiments, the addictive or compulsive behavior is a food addiction. In another embodiment, the addictive or compulsive behavior is binge eating. In certain embodiments, the addictive or compulsive disorder is an obsessive-compulsive disorder.

In certain embodiments, the subject is addicted to an addictive or compulsive behavior or suffers from an impulse-control disorder. In some embodiments, the subject suffers from a primary impulse-control disorder, i.e., an impulse-control disorder in which the disorder is a primary disorder rather than a disorder that is either iatrogenic (secondary to medical treatment) or that is secondary to another primary disease or disorder. Addictive or compulsive behaviors that are primary impulse-control disorders include the following: binge eating, pathological gambling, pathological use of electronic devices, pathological use of electronic video games, pathological use of electronic communication devices, pathological use of cellular telephones, addiction to pornography, sex addiction, compulsive spending, anorexia, bulimia, intermittent explosive disorder, kleptomania, pyromania, trichotillomania, compulsive over-exercising, and compulsive over-working. In some embodiments, the addictive or compulsive behavior is binge eating. In another embodiment, the subject has an obsessive-compulsive disorder.

Another embodiment provides a method for decreasing inflammation, the method comprising administering a PDE 7 inhibitor (e.g., a compound of Structure (I) or Structure (II)) to a subject in need thereof. In some embodiments, the inflammation is in lymphocytes. In some embodiments, the inflammation is in the brain.

One embodiment provides a method of treating neurodegenerative diseases (e.g., Parkinson's disease and Lewy body dementia), central nervous system disorders (e.g., Alzheimer's disease), cancer (e.g., kidney, breast, prostate, blood, papillary, and lung cancers, acute myelogenous leukemia, and multiple myeloma), or inflammatory diseases (e.g., leprosy, Crohn's disease, amyotrophic lateral sclerosis, rheumatoid arthritis, and ankylosing spondylitis), the method comprising administering a compound of Structure (I) or (II) to a subject in need thereof.

One embodiment provides a method for treating autoimmune encephalitis, the method comprising administering a PDE 7 inhibitor (e.g., a compound of Structure (I) or Structure (II)) to a subject in need thereof.

Yet another embodiment provides a method for treating schizophrenia, the method comprising administering a PDE 7 inhibitor (e.g., a compound of Structure (I) or Structure (II)) to a subject in need thereof.

One embodiment provides a method of enhancing central nervous system (CNS) function, the method comprising administering a PDE 7 inhibitor (e.g., a compound of Structure (I) or Structure (II)) to a subject in need thereof. In some embodiments, the CNS function is brain function. In some embodiments, the CNS function is spinal cord function. In some embodiments, the CNS function is nerve cell function.

Some embodiments provide treatment for a psychological disease or condition, the method comprising administering a PDE 7 inhibitor (e.g., a compound of Structure (I) or Structure (II)) to a subject in need thereof. In some embodiments, the disease or condition is anxiety, depression, suicidal thoughts, social phobias, mania, specific phobias (e.g., agoraphobia, claustrophobia, etc.), panic disorders, obsessive compulsive disorder (OCD), post-traumatic stress disorder (PTSD), paranoia, dissociation disorders, dissociative disorders, eating disorders (e.g., anorexia, bulimia nervosa, binge eating), psychosis, or combinations thereof. In some embodiments, the disease or condition is a behavioral and emotional disorders (e.g., oppositional defiant disorder, conduct disorder, attention deficit hyperactivity disorder, etc.), an anxiety disorder, a bipolar affective disorder, or a combination thereof.

In some embodiments, the disease or disorder is drug-induced psychosis, schizophrenia, mood disorders, or a combination thereof. In some embodiments, the treatment further comprises ameliorating symptoms that include delusions, hallucinations, though disorders, social withdrawal, lack of motivation, impaired thinking, impaired memory, confused thinking, or combinations thereof. In some embodiments, the treatment further comprises ameliorating symptoms that include disruptive thinking, disruptive emotions, a distorted perception of reality.

The present disclosure also provides a method of treating or preventing an addiction, comprising providing to a subject having an addiction, an inhibitor of a phosphodiesterase 7 (PDE 7) and an additional therapeutic agent, wherein each of the PDE 7 inhibitor and the additional therapeutic agent contribute to the effective treatment or prevention of the addiction. Additional therapeutic agents include, e.g., opioid antagonists, mixed opioid partial agonist/antagonists, antidepressants, antiepileptics, antiemetics, corticotrophin-releasing factor-1 (CRF-1) receptor antagonists, selective serotonin-3 (5-HT3) antagonists, 5-HT2A/2C antagonists, cannabinoid-1 (CB1) receptor antagonists and dopamine receptor agonists or other dopaminergic agents.

In some embodiments, the treatment further comprises additional medication. In some embodiments, the treatment further comprises cognitive behavioral therapy. In some embodiments, the treatment further comprises psychological support (e.g., counseling).

Exemplary opioid antagonists include naltrexone and nalmefene. Exemplary antidepressants include fluoxetine, mirtazapine, and bupropion. Exemplary antiepileptics include topiramate, levetiracetam, and gabapentin. Antalarmin is an exemplary CRF-1 receptor antagonist. Ondensetrom is an exemplary selective serotonin-3 (5-HT3) antagonist. Exemplary cannabinoid-1 (CB1) receptor antagonists are rimonabant and tanarabant. Buprenorphine is an exemplary mixed opioid agonist/antagonist. Exemplary opioid agonists include morphine, methadone, fentanyl, sufentanil and heroin.

Exemplary dopaminergic agents include, for example, levodopa (also referred to as “L-dopa”), carbidopa, and dopamine receptor agonists and precursors such as bromocriptine, pergolide, pramipexole, ropinirole, cabergoline, apomorphine, lisuride, rotigotine and quinagolide, as well as fenoldopam, which is selective for dopamine receptor D1.

The examples and preparations provided below further illustrate and exemplify the compounds of the present disclosure and methods of preparing and testing such compounds. It is to be understood that the scope of the present disclosure is not limited in any way by the scope of the following examples and preparations. In the following examples, and throughout the specification and claims, molecules with a single stereocenter, unless otherwise noted, exist as a racemic mixture. Those molecules with two or more stereocenters, unless otherwise noted, exist as a racemic mixture of diastereomers. Single enantiomers/diastereomers may be obtained by methods known to those skilled in the art.

### Methods of Preparation

Disclosed herein are novel methods of preparing compounds of Structure (I) and/or Structure (II). Specifically, it has been discovered that synthetic intermediate compounds can be prepared using a novel method that results in a high yield and produces solid material (e.g., crystalline solid).

Accordingly, one embodiment provides a method for preparing a compound, the method comprising contacting a hydrazine compound (or a salt thereof) having the following structure:

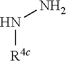

wherein:
R^{4c} is alkyl, aryl, arylalkyl, cycloalkyl, cycloalkylalkyl, heterocyclyl, heterocyclylalkyl, heteroaryl, or heteroarylalkyl;
with an ester compound (or a salt thereof) having the following structure:

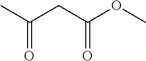

thereby forming a pyrazole compound having the following structure:

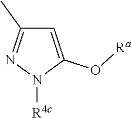

wherein:
R^{a} is hydrogen or —CH₃, as a stereoisomer, enantiomer, or tautomer thereof or a mixture thereof; or a pharmaceutically acceptable salt or solvate thereof.

In some embodiments, R^{4c} is C₁-C₆ alkyl. In certain embodiments, R^{4c} is C₆-C₁₀ aryl. In some embodiments, R^{4c} is C₆-C₁₀ aryl-C₁-C₆ alkyl (i.e., the aryl component has 6-10 carbons and the alkyl component has 1-6 carbons). In some embodiments, R^{4c} is C₃-C₈ cycloalkyl. In certain embodiments, R^{4c} is C₃-C₈ cycloalkyl-C₁-C₆ alkyl. In some embodiments, R^{4c} is 4-8 membered heterocyclyl. In certain embodiments, R⁴, is 4-8 membered heterocyclyl-C₁-C₆ alkyl (i.e., the heterocyclyl component has 4-6 members and the alkyl component has 1-6 carbons). In some embodiments, R^{4c} is 5-12 membered heteroaryl. In certain embodiments, R^{4c} is 5-12 membered heteroaryl-C₁-C₆ alkyl.

In some embodiments, Rₐ is hydrogen. In certain embodiments, Rₐ is —CH₃.

In some embodiments, the hydrazine compound, the ester compound, and a polar aprotic solvent (e.g., tetrahydrofuran) are combined to form a first mixture. In some embodiments, the first mixture is substantially free of acetic acid (e.g., no acetic acid is detected by methods known in the art—e.g., ¹H NMR). In some embodiments, the first mixture is free of acetic acid. In some embodiments, the first mixture is substantially free of water (e.g., no water is detected by methods known in the art—e.g., ¹H NMR). In some embodiments, the first mixture is not heated (e.g., the first mixture is left at room or ambient temperature). In some embodiments, the first mixture is not heated at a temperature above 30° C., 27° C., 25° C., 22° C., or 20° C. In some embodiments, the first mixture remains at a temperature below 30° C., 27° C., 25° C., 22° C., or 20° C. In some embodiments, the first mixture is mixed for more than 10 minutes, more than 30 minutes, more than 1 hour, more than 2 hours, more than 8 hours, more than 12 hours, or more than 16 hours. In some embodiments, the first mixture is mixed for less than 10 minutes, more than 30 minutes, more than 1 hour, more than 2 hours, more than 8 hours, more than 12 hours, or more than 16 hours.

In some embodiments, the method further comprises drying the pyrazole compound. In some embodiments, the pyrazole compound is dried under reduced pressure (e.g., less than 760 torr or less than 380 torr). The term “dried” refers to the removal of solvents or other volatile impurities and does not include the desired reaction product or reactants (e.g., the pyrazole compound, the ester compound, or the hydrazine compound).

In some embodiments, the pyrazole compound is contacted with phosphorus oxychloride. In some embodiments, the pyrazole compound is mixed with phosphorus oxychloride and dimethyl formamide to form a second mixture. In some embodiments, the pyrazole compound is thereby converted to an aldehyde compound having the following structure:

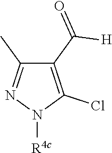

as a stereoisomer, enantiomer, or tautomer thereof or a mixture thereof; or a pharmaceutically acceptable salt or solvate thereof.

In some embodiments, the internal temperature of the second mixture is increased to between 25-35° C. In some embodiments, the second mixture is heated at a temperature between 25-35° C. In some embodiments, the internal temperature of the second mixture is increased to between 28-32° C. In some embodiments, the second mixture is heated at a temperature between 28-32° C. In certain embodiments, the internal temperature of the second mixture is increased to between 29-31° C. In some embodiments, the second mixture is heated at a temperature between 29-31° C.

In some embodiments, the internal temperature of the second mixture is increased to between 65-80° C. In some embodiments, the second mixture is heated at a temperature between 65-80° C. In some embodiments, the internal temperature of the second mixture is increased to between 68-77° C. In some embodiments, the second mixture is heated at a temperature between 68-77° C. In certain embodiments, the internal temperature of the second mixture is increased to between 69-76° C. In some embodiments, the second mixture is heated at a temperature between 69-76° C.

In some embodiments, the second mixture is mixed and/or heated for more than 10 minutes, more than 30 minutes, more than 1 hour, more than 2 hours, more than 8 hours, more than 12 hours, or more than 16 hours. In some embodiments, the second mixture is mixed and/or heated for less than 10 minutes, more than 30 minutes, more than 1 hour, more than 2 hours, more than 8 hours, more than 12 hours, or more than 16 hours.

In some embodiments, the second mixture is added to a solution of aqueous potassium hydroxide to form a third mixture. In certain embodiments, the solution of aqueous potassium hydroxide is cooled to a temperature between −7 to 7° C. In some embodiments, aqueous potassium hydroxide is cooled at a temperature between −7 to 7° C. In some embodiments, the solution of aqueous potassium hydroxide is cooled to a temperature between −5 to 5° C. In some embodiments, aqueous potassium hydroxide is cooled at a temperature between −5 to 5° C.

In some embodiments, the pH of the third mixture is adjusted to pH 6 to 8. In certain embodiments, the pH of the third mixture is adjusted to pH 6 to 8 by adding potassium hydroxide to the third mixture.

In some embodiments, the method of preparation includes at least one of the following reactions Step 1 and Step 2:

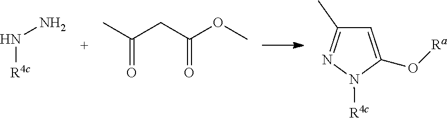

### Step 1

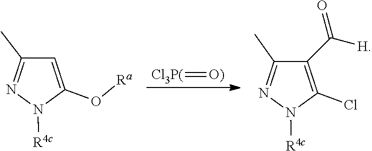

### Step 2

wherein:
R^{a} is hydrogen or —CH₃; and
R^{4c} is alkyl, aryl, arylalkyl, cycloalkyl, cycloalkylalkyl, heterocyclyl, heterocyclylalkyl, heteroaryl, or heteroarylalkyl; and
each compound shown is as a stereoisomer, enantiomer, or tautomer thereof or a mixture thereof; or a pharmaceutically acceptable salt or solvate thereof.

In some embodiments, R^{4c} is C₁-C₆ alkyl. In certain embodiments, R^{4c} is C₆-C₁₀ aryl. In some embodiments, R^{4c} is C₆-C₁₀ aryl-C₁-C₆ alkyl (i.e., the aryl component has 6-10 carbons and the alkyl component has 1-6 carbons). In some embodiments, R^{4c} is C₃-C₈ cycloalkyl. In certain embodiments, R^{4c} is C₃-C₈ cycloalkyl-C₁-C₆ alkyl. In some embodiments, R^{4c} is 4-8 membered heterocyclyl. In certain embodiments, R^{4c} is 4-8 membered heterocyclyl-C₁-C₆ alkyl (i.e., the heterocyclyl component has 4-6 members and the alkyl component has 1-6 carbons). In some embodiments, R^{4c} is 5-12 membered heteroaryl. In certain embodiments, R^{4c} is 5-12 membered heteroaryl-C₁-C₆ alkyl.

In some embodiments, Step 1 is carried out in the presence of a polar aprotic solvent (e.g., tetrahydrofuran). In some embodiments, Step 1 is carried out while the reaction mixture is substantially free of acetic acid (e.g., no acetic acid is detected by methods known in the art—e.g., ¹H NMR). In some embodiments, Step 1 is carried out while the reaction mixture is free of acetic acid. In some embodiments, Step 1 is carried out while the reaction mixture is substantially free of water (e.g., no water is detected by methods known in the art—e.g., ¹H NMR). In some embodiments, Step 1 is not heated (e.g., Step 1 left at room or ambient temperature). In some embodiments, Step 1 is not heated at a temperature above 30° C., 27° C., 25° C., 22° C., or 20° C. In some embodiments, the first mixture remains at a temperature below 30° C., 27° C., 25° C., 22° C., or 20° C. In some embodiments, Step 1 is mixed for more than 10 minutes, more than 30 minutes, more than 1 hour, more than 2 hours, more than 8 hours, more than 12 hours, or more than 16 hours. In some embodiments, Step 1 is mixed for less than 10 minutes, more than 30 minutes, more than 1 hour, more than 2 hours, more than 8 hours, more than 12 hours, or more than 16 hours.

In some embodiments, Step 1 further comprises drying the pyrazole compound. In some embodiments, the pyrazole compound is dried under reduced pressure (e.g., less than 760 torr or less than 380 torr). The term “dried” refers to the removal of solvents or other volatile impurities and does not include the desired reaction product or reactants (e.g., the pyrazole compound, the ester compound, or the hydrazine compound).

In some embodiments, the method comprises Step 1 and Step 2. In some embodiments, Step 2 is performed after Step 1.

In some embodiments, Step 2 is carried out in the presence of dimethyl formamide as a solvent. In some embodiments, Step 2 is carried out while the reaction mixture is substantially free of water (e.g., no water is detected by methods known in the art—e.g., ¹H NMR).

In some embodiments, the internal temperature of the reaction mixture for Step 2 is increased to between 25-35° C. In some embodiments, the reaction mixture for Step 2 is heated at a temperature between 25-35° C. In some embodiments, the internal temperature of the reaction mixture for Step 2 is increased to between 28-32° C. In some embodiments, the reaction mixture for Step 2 is heated at a temperature between 28-32° C. In certain embodiments, the internal temperature of the reaction mixture for Step 2 is increased to between 29-31° C. In some embodiments, the reaction mixture for Step 2 is heated at a temperature between 29-31° C.

In some embodiments, the internal temperature of the reaction mixture for Step 2 is increased to between 65-80° C. In some embodiments, the reaction mixture for Step 2 is heated at a temperature between 65-80° C. In some embodiments, the internal temperature of the reaction mixture for Step 2 is increased to between 68-77° C. In some embodiments, the reaction mixture for Step 2 is heated at a temperature between 68-77° C. In certain embodiments, the internal temperature of the reaction mixture for Step 2 increased to between 69-76° C. In some embodiments, the reaction mixture for Step 2 is heated at a temperature between 69-76° C.

In some embodiments, the reaction mixture for Step 2 is mixed and/or heated for more than 10 minutes, more than 30 minutes, more than 1 hour, more than 2 hours, more than 8 hours, more than 12 hours, or more than 16 hours. In some embodiments, the reaction mixture for Step 2 is mixed and/or heated for less than 10 minutes, more than 30 minutes, more than 1 hour, more than 2 hours, more than 8 hours, more than 12 hours, or more than 16 hours.

In some embodiments, the reaction mixture for Step 2 is added to a solution of aqueous potassium hydroxide to form a third mixture. In certain embodiments, the solution of aqueous potassium hydroxide is cooled to a temperature between −7 to 7° C. In some embodiments, aqueous potassium hydroxide is cooled at a temperature between −7 to 7° C. In some embodiments, the solution of aqueous potassium hydroxide is cooled to a temperature between −5 to 5° C. In some embodiments, aqueous potassium hydroxide is cooled at a temperature between −5 to 5° C.

In some embodiments, the pH of the third mixture is adjusted to pH 6 to 8. In certain embodiments, the pH of the third mixture is adjusted to pH 6 to 8 by adding potassium hydroxide to the third mixture.

In some embodiments, R^{a} is hydrogen. In certain embodiments, R^{a} is CH₃.

In some embodiments, R^{4c} is C₁-C₆ alkyl, C₆-C₁₀ aryl, C₁₀-C₁₄ arylalkyl (e.g., a C₆-C₁₀ aryl system connected to the remainder of the molecule via a C₁-C₄ alkylene chain), C₃-C₁₀ cycloalkyl, C₇-C₁₄ cycloalkylalkyl (e.g., a C₃-C₁₀ cycloalkyl connected to the remainder of the molecule via a C₁-C₄ alkylene chain), a 4-10 membered heterocyclyl, a 4-10 membered heterocyclyl-C₁-C₄ alkyl, a 5-12 membered heteroaryl, or a 5-12 membered heteroaryl-C₁-C₄ alkyl. In some embodiments, R^{4c} is optionally substituted with one or more substituents selected from the group consisting of C₁-C₆ alkyl, halo, cyano, or C₁-C₆ haloalkyl.

In some embodiments, R^{4c} is —CH₂CH(CH₃)₂, —CH₂C(CH₃)₃,

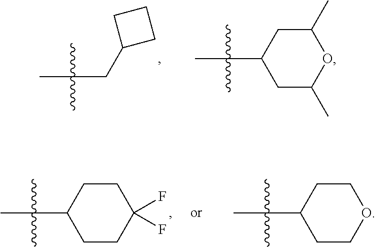

In certain embodiments, R^{4c} is —CH₂CH(CH₃)₂. In some embodiments, R^{4c} is —CH₂C(CH₃)₃. In some embodiments, R^{4c} is

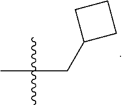 [SMILES: *CC1CCC1]

In certain embodiments, R⁴, is

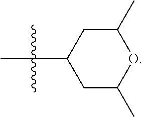 [SMILES: *C1CC(C)OC(C)C1]

In some embodiments, R^{4c} is

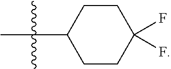 [SMILES: *C1CCC(F)(F)CC1]

In certain embodiments, R^{4c} is

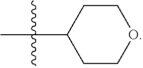 [SMILES: *C1CCOCC1]

### EXAMPLES

Any of the reaction schemes can be modified at any step to add and/or modify a substituent as appropriate during any stage of the overall synthesis of desired compounds.

It will also be appreciated by those skilled in the art that in the processes for preparing the compounds described herein the functional groups of intermediate compounds may need to be protected by suitable protecting groups. Such functional groups include, but are not limited to, hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups are optionally added or removed in accordance with standard techniques, which are known to one skilled in the art and as described herein. The use of protecting groups is described in detail in Green, T. W. and P. G. M. Wutz, Protective Groups in Organic Synthesis (1999), 3Rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this disclosure may not possess pharmacological activity as such, they may be administered to a mammal and thereafter metabolized in the body to form compounds of the disclosure which are pharmacologically active. Such derivatives may therefore be described as “prodrugs”. Prodrugs of compounds of this disclosure are included within the scope of embodiments of the disclosure.

### General Procedures

All proton NMR experiments were recorded on a Bruker NEO Spectrometer equipped with a BBFO probe at 400 MHz. Deuterated solvents contained less than 0.05% v/v tetramethylsilane which was used as the reference signal (set at 0.00 ppm). When deuterated solvents did not contain tetramethylsilane, the residual nondeuterated solvent peaks were used as a reference signal, as per published guidelines (J. Org. Chem. 1997, 62(21), 7512-7515). Chemical shifts are expressed in parts per million (ppm, 6 units).

Coupling constants are in hertz (Hz). Splitting patterns describe apparent multiplicities and are designated as broad singlet (bs), s (singlet), d (doublet), t (triplet), q (quartet), m (multiplet), qt (quintuplet) or brs (broad singlet).

LC/MS analyses were performed on an Agilent Technologies UHPLC 1290 Infinity II with a G6125 MS detector.

Microwave reactions were conducted with a Monowave 300 by Anton Paar GmbH using standard protocols.

### Abbreviations

° C. (degree Celsius); ¹H NMR (proton Nuclear Magnetic Resonance); ACN (acetonitrile); AcOH (acetic acid); DCE (dichloroethane); DCM (dichloromethane); DIPEA (N,N-diisopropylethylamine); DMF (N,N-dimethylformamide); DMSO-d₆ (deuterated dimethylsulfoxide); eq (equivalent); EtOAc (ethyl acetate); g (gram); h or hr (hour); HPLC (High Performance Liquid Chromatography); LC-MS (Liquid Chromatography Mass Spectrometry); MeOH (methanol); mg (milligram); min (minute); mL (milliliter); mmol (millimole); Pd(OAc)₂ (palladium(II) acetate); TFA (trifluoroacetic acid); THF (tetrahydrofuran); TLC (Thin Layer Chromatography).

### Synthetic Example 1

### Synthesis of (1r,3r)-3-morpholinocyclobutan-1-amine bis(2,2,2-trifluoroacetate) (Intermediate 1)

### Step 1: Synthesis of tert-butyl ((1r,3r)-3-morpholinocyclobutyl)carbamate

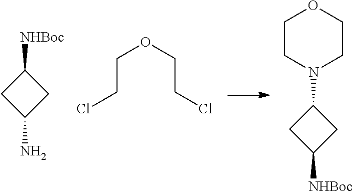

To a slurry consisting of tert-butyl ((1r,3r)-3-aminocyclobutyl)carbamate (1 g, 5 mmol), sodium bicarbonate (2 g, 0.03 mol) and sodium iodide (2 g, 0.01 mol) in EtOH (10 mL) was added 1-chloro-2-(2-chloroethoxy)ethane (0.6 mL, 5 mmol). The reaction mixture was stirred at 74° C. for 36 hrs before being concentrated under vacuum. The solid residue was diluted with H₂O (100 mL) and 50 mL sat aqueous sodium thiosulfate (Na₂S₂O₃) then extracted with EtOAc (3×100 mL). The organic extracts were dried with sodium sulfate and concentrated to yield tert-butyl ((1r,3r)-3-morpholinocyclobutyl)carbamate (1.5 g, 100%) as a brown oil.

### Step 2: Synthesis of (1r,3r)-3-morpholinocyclobutan-1-amine bis(2,2,2-trifluoroacetate)

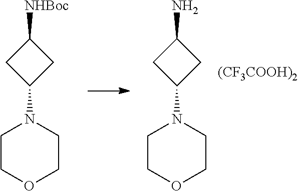

Crude tert-butyl ((1r,3r)-3-morpholinocyclobutyl)carbamate (230 mg, 0.897 mmol) was dissolved in DCM (2 mL) and treated with trifluoroacetic acid (0.34 mL, 4.49 mmol). After stirring at RT for 1 hr. the reaction mixture was concentrated to yield (1r,3r)-3-morpholinocyclobutan-1-amine bis(2,2,2-trifluoroacetate (345 mg, 100%) as a brown oil. The crude was used in the next reaction without further purification.

### Synthetic Example 2

### Synthesis of (1r,4r)-3-morpholinocyclohexan-1-amine bis(2,2,2-trifluoroacetate) (Intermediate 2)

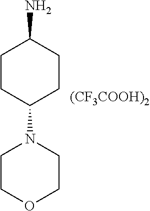

(1r,4r)-3-morpholinocyclohexan-1-amine bis(2,2,2-trifluoroacetate) was synthesized using the procedures described in Synthetic Example 1 using tert-butyl ((1r,4r)-4-aminocyclohexyl) carbamate.

### Synthetic Example 3

### Synthesis of 1-(4-((1r,3r)-3-aminocyclobutyl)piperazin-1-yl)ethan-1-one bis(2,2,2-trifluoroacetate) (Intermediate 3)

### Step 1: Synthesis of tert-butyl ((1r,3r)-3-(4-benzylpiperazin-1-yl)cyclobutyl)carbamate

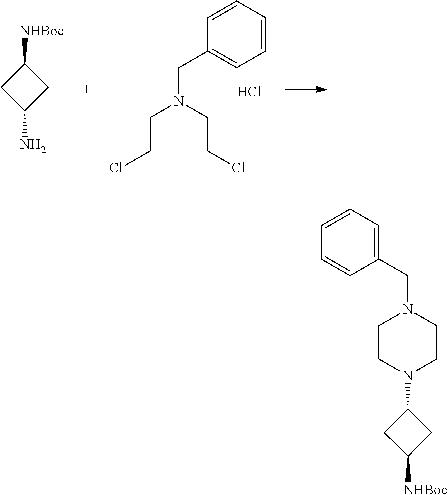 [SMILES: CC(C)(C)OC(=O)N[C@H]1C[C@H](N)C1 ; ClCCN(CCCl)Cc1ccccc1 ; Cl ; CC(C)(C)OC(=O)N[C@H]1C[C@H](N2CCN(Cc3ccccc3)CC2)C1]

Tert-butyl ((1r,3r)-3-(4-benzylpiperazin-1-yl)cyclobutyl)carbamate was synthesized according to the procedure described in Step 1 of Synthetic Example 1 using N-benzyl-1-chloro-N-(chloromethyl)methanamine HCl salt.

### Step 2: Synthesis of tert-butyl ((1r,3r)-3-(piperazin-1-yl)cyclobutyl)carbamate diacetate

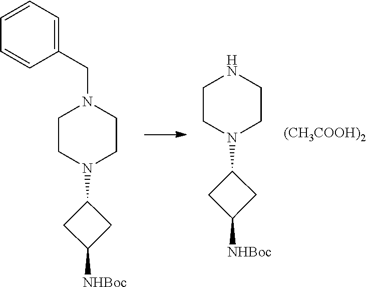

To a solution of tert-butyl ((1r,3r)-3-(4-benzylpiperazin-1-yl)cyclobutyl)carbamate (2 g, 6 mmol) in EtOH (20 mL) were added Pd(OH)₂ (0.8 g, 6 mmol) and acetic acid (3.2 mL, 60 mol). After degassing with N₂, the mixture was stirred under 55 psi of H₂ for 2 hrs. The reaction mixture was filtered through a pad of diatomaceous earth (e.g., Celite®), washed thoroughly with MeOH (3 5×20 mL) and the solvent evaporated under vacuum to yield tert-butyl ((1r,3r)-3-(piperazin-1-yl)cyclobutyl)carbamate diacetate (1.8 g, 80%) as a colorless oil that was used in the next reaction without further purification.

### Step 3: Synthesis of tert-butyl ((1r,3r)-3-(4-acetylpiperazin-1-yl)cyclobutyl)carbamate acetate

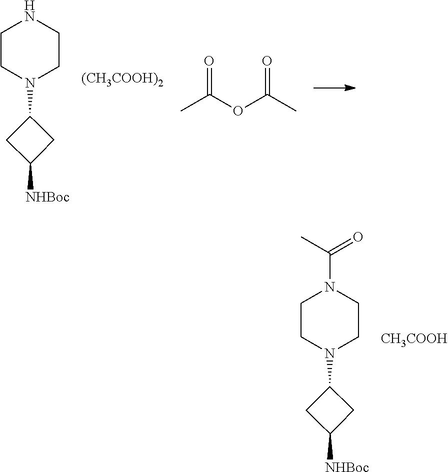

To crude tert-butyl ((1r,3r)-3-(piperazin-1-yl)cyclobutyl)carbamate diacetate (200 mg, 0.533 mmol) suspended in THF (10 mL) were added acetic anhydride (251 μL, 2.66 mmol) and DIPEA (278 μL, 1.60 mmol). The reaction mixture was stirred at 50° C. for 1 hr. before MeOH (5 mL) was added to quench the excess anhydride. Evaporation of the solution afforded tert-butyl ((1r,3r)-3-(4-acetylpiperazin-1-yl)cyclobutyl)carbamate acetate (190 mg, 100%) as a colorless oil.

### Step 4: Synthesis of 1-(4-((1r,3r)-3-aminocyclobutyl)piperazin-1-yl)ethan-1-one bis(2,2,2-trifluoroacetate)

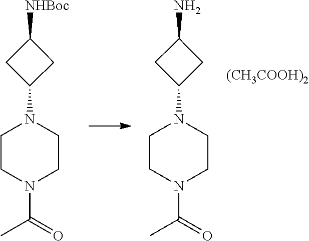

1-(4-((1r,3r)-3-aminocyclobutyl)piperazin-1-yl)ethan-1-one bis(2,2,2-trifluoroacetate) was synthesized using the procedure described in Step 2 of Synthetic Example 1.

### Synthetic Example 4

### Synthesis of 1-(4-((1r,4r)-4-aminocyclohexyl)piperazin-1-yl)ethan-1-one bis(2,2,2-trifluoroacetate) (Intermediate 4)

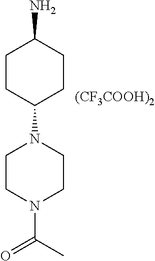

1-(4-((1r,4r)-4-aminocyclohexyl)piperazin-1-yl)ethan-1-one bis(2,2,2-trifluoroacetate) was synthesized using the procedures described in Synthetic Example 3 using tert-butyl ((1r,4r)-4-aminocyclohexyl)carbamate.

### Synthetic Example 5

### Synthesis of (1r,3r)-3-(4-cyclopropylpiperazin-1-yl)cyclobutan-1-amine tris(2,2,2-trifluoroacetate) (Intermediate 5)

### Step 1: Synthesis of tert-butyl ((1r,3r)-3-(4-cyclopropylpiperazin-1-yl)cyclobutyl)carbamate

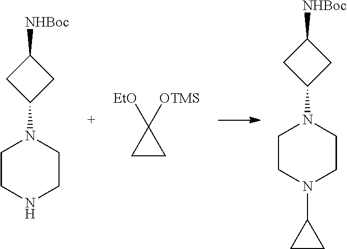

To a solution of tert-butyl ((1r,3r)-3-(piperazin-1-yl)cyclobutyl)carbamate (300 mg, 1.17 mmol) suspended in MeOH (3 mL) and THF (3 mL) was added (1-ethoxycyclopropoxy)trimethylsilane (1.02 g, 1.0 mL, 5.87 mmol) followed by sodium cyanoborohydride (244 mg, 3.88 mmol). The resultant mixture was heated at 60° C. for 14 hrs. After cooling to RT, the mixture was treated with water (20 mL) and the pH adjusted to 10 using 1 N NaOH. The solution was concentrated to −10 mL, extracted with DCM (3×30 mL) and the organic washes dried over sodium sulfate. Evaporation of the solvent afforded crude tert-butyl ((1r,3r)-3-(4-cyclopropylpiperazin-1-yl)cyclobutyl)carbamate (347 mg, 100%) as a light brown solid.

### Step 2: Synthesis of (1r,3r)-3-(4-cyclopropylpiperazin-1-yl)cyclobutan-1-amine tris(2,2,2-trifluoroacetate)

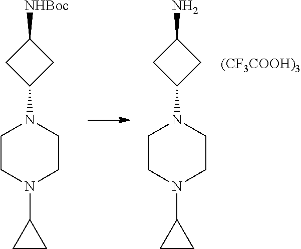

(1r,3r)-3-(4-cyclopropylpiperazin-1-yl)cyclobutan-1-amine tris(2,2,2-trifluoroacetate was synthesized using the procedure described in Step 2 of Synthetic Example 1.

### Synthetic Example 6

### Synthesis of (1r,4r)-4-(4-cyclopropylpiperazin-1-yl)cyclohexan-1-amine tris(2,2,2-trifluoroacetate) (Intermediate 6)

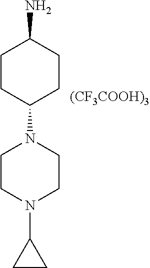

(1r,4r)-4-(4-cyclopropylpiperazin-1-yl)cyclohexan-1-amine tris(2,2,2-trifluoroacetate) was synthesized using the procedures described in Synthetic Example 5.

### Synthetic Example 7

### Synthesis of (1r,4r)-4-(3-(difluoromethoxy)azetidin-1-yl)cyclohexan-1-amine (Intermediate 7)

### Step 1: Synthesis of benzyl ((1r,4r)-4-(3-(difluoromethoxy)azetidin-1-yl)cyclohexyl)carbamate

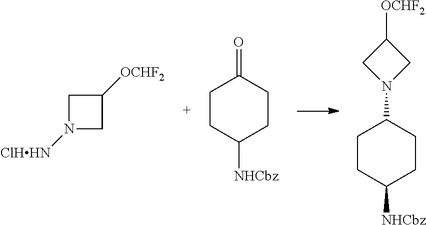

To 3-(difluoromethoxy)azetidine hydrochloride (1.0 g, 6.3 mmol) and benzyl (4-oxocyclohexyl)carbamate (1.5 g, 6.3 mmol) suspended in dichloroethane (16 mL) were added DIPEA (1.1 mL, 6.3 mmol) and acetic acid (0.36 mL, 6.3 mmol). The reaction mixture was stirred at 60° C. for 2 hrs. before sodium triacetoxyborohydride (1.3 g, 6.3 mmol) was added and stirred for an additional 3 hrs. The reaction mixture was diluted with H₂O (100 mL), neutralized with sat. NaHCO₃ (100 mL) and extracted with DCM (2×200 mL). The organic extracts were dried over Na₂SO₄ and concentrated to afford a crude brown oil that was purified by FCC (SiO₂) using a 15-100% (Hep/EtOAc) gradient to yield benzyl ((1r,4r)-4-(3-(difluoromethoxy)azetidin-1-yl)cyclohexyl)carbamate (500 mg, 23%) as a white solid. The cis isomer eluted first with desired trans product eluting in 100% EtOAc.

### Step 2: Synthesis of (1r,4r)-4-(3-(difluoromethoxy)azetidin-1-yl)cyclohexan-1-amine

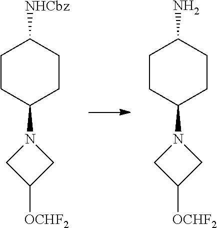 [SMILES: O=C(N[C@H]1CC[C@H](N2CC(OC(F)F)C2)CC1)OCc1ccccc1 ; N[C@H]1CC[C@H](N2CC(OC(F)F)C2)CC1]

A solution consisting of benzyl ((1r,4r)-4-(3-(difluoromethoxy)azetidin-1-yl)cyclohexyl) carbamate (500 mg, 1.41 mmol) and 10% Pd/C (23 mg, 0.22 mmol) in MeOH (20 mL) was stirred under a H₂ atmosphere for 2 hrs. The reaction mixture was filtered thru a pad of diatomaceous earth (e.g., Celite®) and washed thoroughly with MeOH (3×20 mL). The organic washes were concentrated to yield (1r,4r)-4-(3-(difluoromethoxy)azetidin-1-yl)cyclohexan-1-amine (286 mg, 92%) as a colorless oil and used in the next reaction without further purification.

### Synthetic Examples 8-11

### Synthesis of Amine Intermediates 8-11

The following amine intermediates were synthesized using the procedures described in Synthetic Example 7.

| Example | | |
|---|---|---|
| Number | Name | Structure |
| 8 | (1r,4r)-4-(3-methoxy- azetidin-1-yl)cyclohexan- 1-amine (Intermediate 8) | 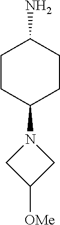 [SMILES: COC1CN([C@H]2CC[C@H](N)CC2)C1] |
| 9 | 1-((1r,4r)-4-aminocyclo- hexyl)azetidine-3-carbo- nitrile (Intermediate 9) | 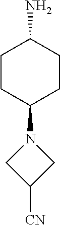 [SMILES: N#CC1CN([C@H]2CC[C@H](N)CC2)C1] |
| 10 | (1r,4r)-4-(3,3-difluoro- pyrrolidin-1-yl)cyclo- hexan-1-amine (Inter- mediate 10) | 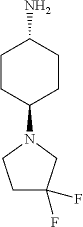 [SMILES: N[C@H]1CC[C@H](N2CCC(F)(F)C2)CC1] |
| 11 | (1r,4r)-4-(3,3-difluoro- azetidin-1-yl)cyclohexan- 1-amine (Intermediate 11) | 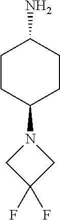 [SMILES: N[C@H]1CC[C@H](N2CC(F)(F)C2)CC1] |

### Synthetic Example 12

### Synthesis of (1r,4r)-4-(4-(2,2-difluoroethyl)piperazin-1-yl)cyclohexan-1-amine tris(2,2,2-trifluoroacetate) (Intermediate 12)

### Step 1: Synthesis of tert-butyl ((1r,4r)-4-(4-(2,2-difluoroethyl)piperazin-1-yl)cyclohexyl) carbamate

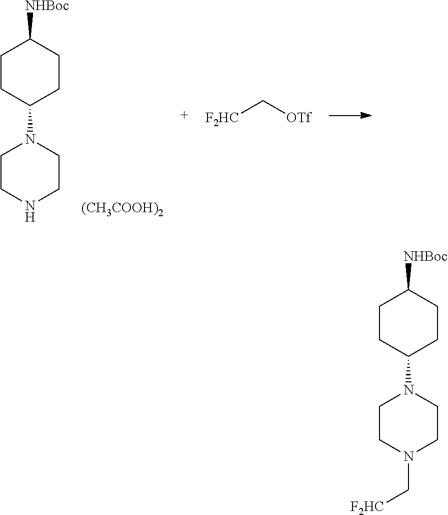

To a suspension of tert-butyl ((1r,4r)-4-(piperazin-1-yl)cyclohexyl)carbamate diacetate (550 mg, 1.36 mmol) in DCM (5 mL) were added DIPEA (1 mL) and 2,2-difluoroethyl trifluoromethanesulfonate (1.46 g, 6.82 mmol). The reaction mixture turned homogenous after heating at 40° C. for 5 min. After stirring at 40° C. for an additional 1 hr, MeOH (2 mL) was added to quench the excess triflate and the solution concentrated under vacuum to yield tert-butyl ((1r,4r)-4-(4-(2,2-difluoroethyl)piperazin-1-yl)cyclohexyl)carbamate (1.3 g, 100%) as a brown oil. The crude was used in the next reaction without further purification.

### Step 2: Synthesis of (1r,4r)-4-(4-(2,2-difluoroethyl)piperazin-1-yl)cyclohexan-1-amine tris(2,2,2-trifluoroacetate)

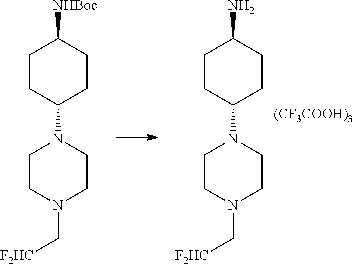

(1r,4r)-4-(4-(2,2-difluoroethyl)piperazin-1-yl)cyclohexan-1-amine tris(2,2,2-trifluoroacetate) was synthesized using the procedure described in Step 2 of Synthetic Example 1.

### Synthetic Example 13

### Synthesis of (1r,4r)-4-(4-(2,2,2-trifluoroethyl)piperazin-1-yl)cyclohexan-1-amine tris(2,2,2-trifluoroacetate) (Intermediate 13)

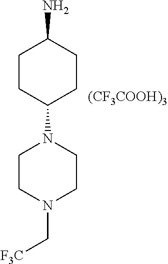

(1r,4r)-4-(4-(2,2,2-trifluoroethyl)piperazin-1-yl)cyclohexan-1-amine tris(2,2,2-trifluoroacetate) was synthesized using the procedures described in Synthetic Example 12.

### Synthetic Example 14

### Synthesis (1r,4r)-4-(4-isopropylpiperazin-1-yl)cyclohexan-1-amine tris (2,2,2-trifluoroacetate) (Intermediate 14)

### Step 1: Synthesis of tert-butyl ((1r,3r)-3-(4-isopropylpiperazin-1-yl)cyclohexyl)carbamate

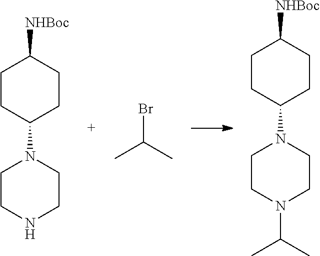

To a suspension of tert-butyl ((1r,3r)-3-(piperazin-1-yl)cyclohexyl)carbamate (300 mg, 1.17 mmol) in THF (3 mL) were added bromopropane (1 mL) and DIPEA (1 mL). The reaction mixture was heated in the microwave reactor at 100° C. for 30 min. then diluted with water (20 mL) and extracted with EtOAc (2×20 mL). After drying over Na₂SO₄, the organic washes were concentrated to yield tert-butyl ((1r,3r)-3-(4-isopropylpiperazin-1-yl)cyclohexyl)carbamate (380 mg, 100%) as a brown oil. The crude oil was used in the next reaction without further purification.

### Step 2: Synthesis of (1r,4r)-4-(4-isopropylpiperazin-1-yl)cyclohexan-1-amine tris (2,2,2-trifluoroacetate)

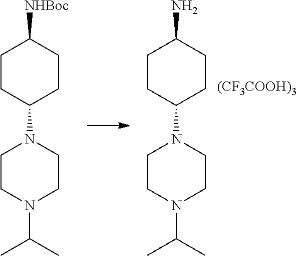

(1r,4r)-4-(4-isopropylpiperazin-1-yl)cyclohexan-1-amine tris (2,2,2-trifluoroacetate) was synthesized using the procedure described in Step 2 of Synthetic Example 1.

### Synthetic Example 15

### Synthesis of 3-(3,3-difluoropyrrolidin-1-yl)cyclobutan-1-amine (Intermediate 15)

### Step 1: Synthesis of benzyl (3-(3,3-difluoropyrrolidin-1-yl)cyclobutyl)carbamate

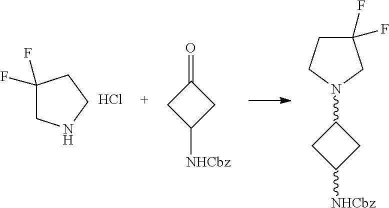 [SMILES: FC1(F)CCNC1 ; Cl ; O=C1CC(NC(=O)OCc2ccccc2)C1 ; O=C(NC1CC(N2CCC(F)(F)C2)C1)OCc1ccccc1]

Benzyl (3-(3,3-difluoropyrrolidin-1-yl)cyclobutyl)carbamate was synthesized using the procedure described in Step 1 of Synthetic Example 7. Cis and trans isomers were not separated during this step.

### Step 2: Synthesis of 3-(3,3-difluoropyrrolidin-1-yl)cyclobutan-1-amine

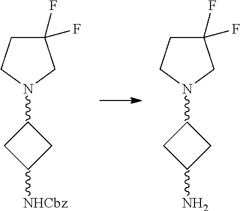 [SMILES: O=C(NC1CC(N2CCC(F)(F)C2)C1)OCc1ccccc1 ; NC1CC(N2CCC(F)(F)C2)C1]

3-(3,3-difluoropyrrolidin-1-yl)cyclobutan-1-amine was synthesized using the procedure described in Step 2 of Synthetic Example 7. Cis and trans isomers could not be separated during this step. The mixture of isomers was used as is for coupling to the corresponding acids.

### Synthetic Example 16

### Synthesis of 1-(4-((1r,4r)-4-aminocyclohexyl)piperazin-1-yl)ethane-1-thione bis(2,2,2-trifluoroacetate) (Intermediate 16)

### Step 1: tert-butyl ((1r,4r)-4-(4-ethanethioylpiperazin-1-yl)cyclohexyl)carbamate

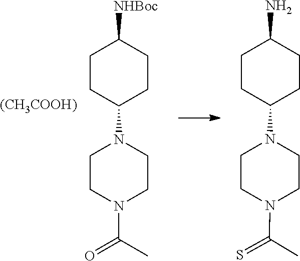 [SMILES: "(CH3COOH)" ; CC(=O)N1CCN([C@H]2CC[C@H](NC(=O)OC(C)(C)C)CC2)CC1 ; CC(=S)N1CCN([C@H]2CC[C@H](N)CC2)CC1]

To a solution of tert-butyl ((1r,4r)-4-(4-acetylpiperazin-1-yl)cyclohexyl)carbamate acetate (395 mg, 1.02 mmol) dissolved in THF (10 mL) was added Lawesson's reagent (414 mg, 1.02 mmol) and stirred at 60° C. for 17 hrs. The reaction mixture was concentrated and purified by FCC (SiO₂) using a 50-100% (Hep/EtOAc) gradient followed by 10% MeOH/DCM to yield tert-butyl ((1r,4r)-4-(4-ethanethioylpiperazin-1-yl)cyclohexyl)carbamate (347 mg, 100%) as a colorless oil.

### Step 2: 1-(4-((1r,4r)-4-aminocyclohexyl)piperazin-1-yl)ethane-1-thione bis(2,2,2-trifluoroacetate)

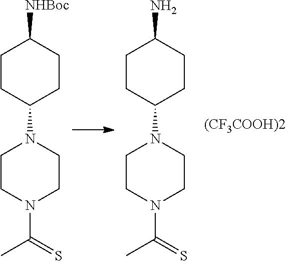 [SMILES: CC(=S)N1CCN([C@H]2CC[C@H](NC(=O)OC(C)(C)C)CC2)CC1 ; CC(=S)N1CCN([C@H]2CC[C@H](N)CC2)CC1]

1-(4-((1r,4r)-4-aminocyclohexyl)piperazin-1-yl)ethane-1-thione bis(2,2,2-trifluoroacetate) was synthesized using the procedure described in Step 2 of Synthetic Example 1.

### Synthetic Example 17

### Synthesis of 5-chloro-1-(cyclobutylmethyl)-3-methyl-1H-pyrazole-4-carbaldehyde (Intermediate 17)

### Step 1: 1-(cyclobutylmethyl)-3-methyl-1H-pyrazol-5-ol

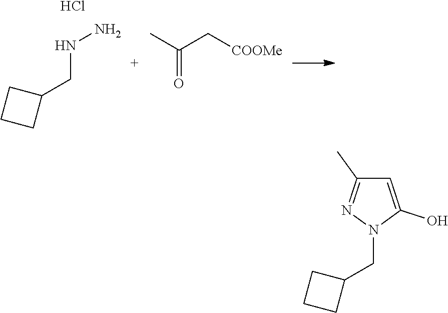

A clean, dry, magnetically stirred, single-necked 500 mL round bottom flask was charged with (cyclobutylmethyl)hydrazine hydrochloride (5.91 g, 43.3 mmoles) and dry tetrahydrofuran (140 mL). The resulting slurry was stirred for 10 minutes before methyl 3-oxobutanoate (5.53 g, 47.6 mmoles, 1.1 eq) was added. THF (10 mL) was used to rinse the residual methyl 3-oxobutanoate into the stirred slurry. After stirring the reaction mixture at room temperature overnight, the reaction mixture had become a clear solution. The reaction was deemed complete by LC-MS and the reaction solution was concentrated under vacuum. The thick oily residue solidified after being placed on high vacuum overnight. The solids were dissolved in THF (25 mL) and ethyl acetate (20 mL) was added slowly. The resulting solids were filtered, washed with ethyl acetate (15 mL) and dried under high vacuum overnight to afford 5.5 g of crude material. Analysis by LC-MS showed both 1-(cyclobutylmethyl)-3-methyl-1H-pyrazol-5-ol and 1-(cyclobutylmethyl)-5-methoxy-3-methyl-4,5-dihydro-1H-pyrazole present. Analysis by qNMR determined the mixture was 76.1 wt % hydroxy (77% yield) and 16.0 wt % of the methoxy analog. Since both hydroxy and methoxy analogs can be converted to the chloro-analog in the next step no further purification was done.

### Step 2: 5-chloro-1-(cyclobutylmethyl)-3-methyl-1H-pyrazole-4-carbaldehyde

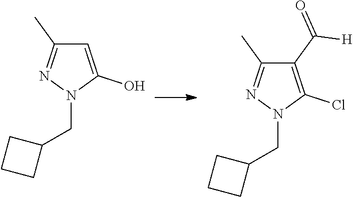

A clean dry, magnetically stirred, two-necked 250 mL round bottom flask was charged with 1.67 g (10 mmol) of isolated material from the previous reaction followed by phosphorous oxychloride (10 mL) to form a thin slurry. To the slurry, dry DMF (1.55 mL, 20 mmoles) was added dropwise. During the addition, the internal temperature increased from 21° C. to 30° C. and a clear yellow solution formed. The reaction solution was slowly heated to an internal temperature of 70° C. to 75° C. and left to stir overnight. After this period, the reaction was deemed complete by HPLC. The mixture was concentrated under vacuum to approximately ⅓ the original volume and the concentrate was slowly added to a cooled (0° C.±5° C.) solution of potassium hydroxide in 100 mL of water, at a rate to keep the temperature below 15° C. Upon completion, the solution pH was found to still be very acidic (around pH 1). Additional KOH solution was slowly added until the pH was near 7. Solids were observed forming throughout neutralization. The resulting slurry was cooled to 5° C. and stirred for 15 minutes. The solids were filtered, washed with water (15 mL), and dried under very high vacuum (0.05 torr) until a constant weight was achieved to afford 1.56 g of the desired compound as an off-white solid.

### Synthetic Examples 18-22

### Synthesis of 3,5-disubstituted pyrazole chloro-aldehydes (Intermediates 18-22)

The following 5-chloro-1-substituted-3-methyl-1H-pyrazole-4-carbaldehyde intermediates were synthesized using the procedures described in Synthetic Example 17.

| Example | | |
|---|---|---|
| Number | Name | Structure |
| 18 | 5-chloro-1-(4,4-difluoro- cyclohexyl)-3-methyl- 1H-pyrazole-4-carbalde- hyde (Intermediate 18) | 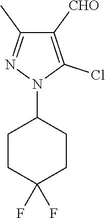 [SMILES: Cc1nn(C2CCC(F)(F)CC2)c(Cl)c1C=O] |
| 19 | 5-chloro-3-methyl-1-neo- pentyl-1H-pyrazole-4- carbaldehyde (Inter- mediate 19) | 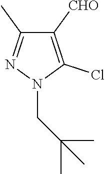 |
| 20 | 5-chloro-1-cyclohexyl-3- methyl-1H-pyrazole-4- carbaldehyde (Inter- mediate 20) | 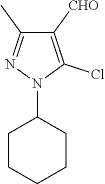 |
| 21 | 5-chloro-3-methyl-1- (tetrahydro-2H-pyran-4- yl)-1H-pyrazole-4-carbal- dehyde (Intermediate 21) | 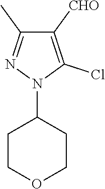 |
| 22 | 5-chloro-1-cyclopropyl- 3-methyl-1H-pyrazole-4- carbaldehyde (Inter- mediate 22) | 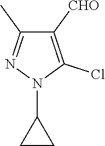 |

### Synthetic Example 23

### Synthesis of 5-chloro-1-((2r,6s)-2,6-dimethyltetrahydro-2H-pyran-4-yl)-3-methyl-1H-pyrazole-4-carbaldehyde

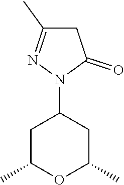

### Step 1: Tert-butyl (E)-2-((2r,6s)-2,6-dimethyltetrahydro-4H-pyran-4-ylidene)hydrazine-1-carboxylate

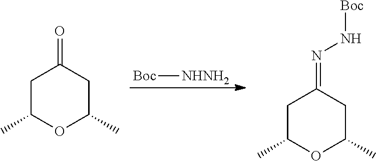

A stirred solution of (2r,6s)-2,6-dimethyltetrahydro-4H-pyran-4-one (10.0 g, 78.0 mmol) and Boc-NHNH₂ (11.3 g, 85.8 mmol) in methanol (100 mL) was stirred at room temperature for overnight. The reaction mixture was concentrated under reduced pressure. The isolated product was used directly next step without further purification (74%). ESI-MS m/z: 243.3 [M+H]⁺

### Step 2: Tert-butyl 2-((2r,6s)-2,6-dimethyltetrahydro-2H-pyran-4-yl)hydrazine-1-carboxylate

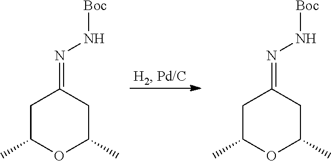

To a solution of tert-butyl (E)-2-((2r,6s)-2,6-dimethyltetrahydro-4H-pyran-4-ylidene)hydrazine-1-carboxylate (11 g, 57.8 mmol) in methanol (110 mL) was added 10% Pd/C (10 g) under nitrogen. The solution was stirred under H atmosphere for 16 h. After completion, the reaction mixture was filtered through a pad of diatomaceous earth (e.g., Celite®) and washed with EtOAc. The filtrate was distilled under reduced pressure to afford the desired compound as a white solid (41%). Identification of the desired compound was confirmed by ¹H NMR.

### Step 3: ((2r,6s)-2,6-dimethyltetrahydro-2H-pyran-4-yl)hydrazine

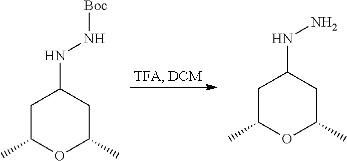

To a stirred solution of tert-butyl 2-((2r,6s)-2,6-dimethyltetrahydro-2H-pyran-4-yl)hydrazine-1-carboxylate (4.0 g, 101 mmol) in DCM (4 mL) was added TFA (3.78 mL) was added drop wise at 0° C. Then the reaction mixture was stirred at RT for 16 h. After completion, the reaction mixture was concentrated under reduced pressure to afford the target compound as a white solid. It was used for the next step without any purification (2.30 g).

### Step 4: 2-((2r,6s)-2,6-dimethyltetrahydro-2H-pyran-4-yl)-5-methyl-2,4-dihydro-3H-pyrazol-3-one

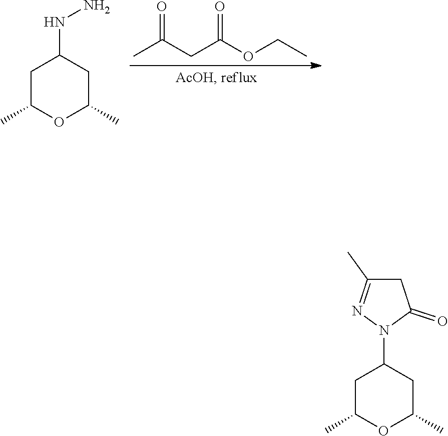

To a stirred solution of ((2r,6s)-2,6-dimethyltetrahydro-2H-pyran-4-yl)hydrazine (2.30 g, 15.9 mmol) in acetic acid (20.0 mL) was added ethyl 3-oxobutanoate (2.08 g, 15.9 mmol) at room temperature. The reaction mixture was stirred under N₂ atmosphere for 16 h at 110° C. After completion, the reaction mixture was concentrated under reduced pressure to get crude residue. The crude residue was purified by silica gel column chromatography (with 90% EtOAc in hexanes) to afford desired compound as a light brown gummy liquid (54%). ESI-MS: m/z 209.16 [M−H]⁻

### Step 5: 5-chloro-1-((2r,6s)-2,6-dimethyltetrahydro-2H-pyran-4-yl)-3-methyl-1H-pyrazole-4-carbaldehyde

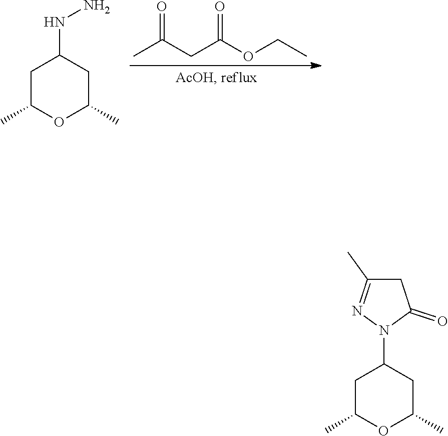

To a stirred solution of 2-((2r,6s)-2,6-dimethyltetrahydro-2H-pyran-4-yl)-5-methyl-2,4-dihydro-3H-pyrazol-3-one (1.80 g, 8.56 mmol) in DMF (15.0 mL) at 0° C. was added POCl₃. (2.62 g, 17.1 mmol). The reaction mixture was stirred under N₂ atmosphere for 16 h at 110° C. Then, the reaction mixture was concentrated under reduced pressure and diluted in cold water. The aqueous layer was basified with 1N NaOH (pH-8-9) and extracted with EtOAc. The organic layer was washed with brine solution, dried over anhydrous Na₂SO₄ and concentrated. The crude residue was purified by silica gel column chromatography (in 8-10% EtOAc in hexanes) to afford the desired compound as a white solid (14%). ESI-MS: m/z m/z: 257.11 [M+H]⁺

### Synthetic Example 24

### Synthesis of 1-(2-chlorophenyl)-3-methyl-1H-thieno[2,3-c]pyrazole-5-carboxylic acid (Intermediate 24)

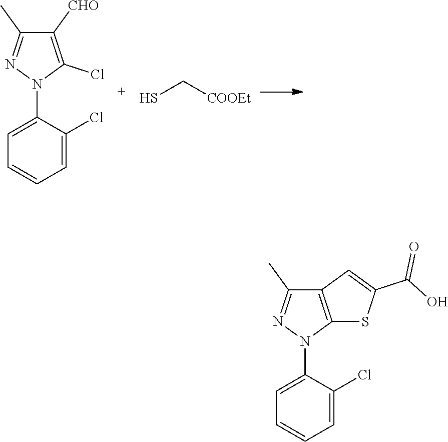 [SMILES: Cc1nn(-c2ccccc2Cl)c(Cl)c1C=O ; CCOC(=O)CS ; Cc1nn(-c2ccccc2Cl)c2sc(C(=O)O)cc12]

To a rb flask with MeOH (8 mL) was added sodium methoxide in MeOH (1.2 mL, 25% wt, 5.10 mmol) followed by ethyl 2-mercaptoacetate (0.2 mL, 2.06 mmol). After stirring at RT for 1 hr, 5-chloro-1-(2-chlorophenyl)-3-methyl-1H-pyrazole-4-carbaldehyde (500 mg, 1.96 mmol) dissolved in THF (2 mL) was added dropwise to the solution and stirred at 60° C. for 14 hrs. After cooling to RT, sodium hydroxide (392 mg, 9.80 mmol) in water (5 mL) was added to the reaction mixture and stirred for 2 hrs. The heterogenous mixture turned homogenous after 2 hrs. After consumption of starting material, the solvent was evaporated, and the residue diluted with water (20 mL). The pH of the solution was slowly adjusted to 2 using 1N HCl under ice bath cooling. The resulting white solid was filtered and dried to yield 1-(2-chlorophenyl)-3-methyl-1H-thieno[2,3-c]pyrazole-5-carboxylic acid (390 mg, 68%).

### Synthetic Examples 25-30

### Synthesis of Carboxylic Acid Intermediates (Intermediates 25-30)

The following thieno[2,3-c]pyrazole carboxylic acid intermediates were synthesized using the procedures described in Synthetic Example 17.

| Example | | |
|---|---|---|
| Number | Name | Structure |
| 25 | 3-methyl-1-(pyridin-2-yl)-1H- thieno[2,3-c]pyrazole-5-carbox- ylic acid (Intermediate 25) | 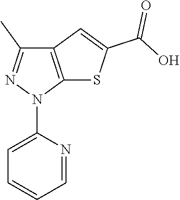 [SMILES: Cc1nn(-c2ccccn2)c2sc(C(=O)O)cc12] |
| 26 | 1-(4,4-difluorocyclohexyl)-3- methyl-1H-thieno[2,3-c] pyrazole-5-carboxylic acid (Intermediate 26) | 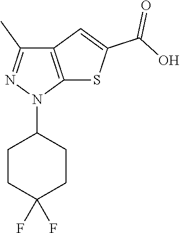 [SMILES: Cc1nn(C2CCC(F)(F)CC2)c2sc(C(=O)O)cc12] |
| 27 | 1-((2r,6s)-2,6-dimethyltetra- hydro-2H-pyran-4-yl)-3- methyl-1H-thieno[2,3-c] pyrazole-5-carboxylic acid (Intermediate 27) | 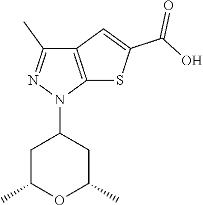 [SMILES: Cc1nn(C2C[C@@H](C)O[C@@H](C)C2)c2sc(C(=O)O)cc12] |
| 28 | 1-(cyclobutylmethyl)-3- methyl-1H-thieno[2,3-c] pyrazole-5-carboxylic acid (Intermediate 28) | 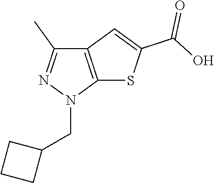 [SMILES: Cc1nn(CC2CCC2)c2sc(C(=O)O)cc12] |
| 29 | 1-isobutyl-3-methyl-1H-thieno [2,3-c]pyrazole-5-carboxylic acid (Intermediate 29) | 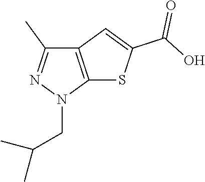 [SMILES: Cc1nn(CC(C)C)c2sc(C(=O)O)cc12] |
| 30 | 3-methyl-1-neopentyl-1H-thieno [2,3-c]pyrazole-5-carboxylic acid (Intermediate 30) | 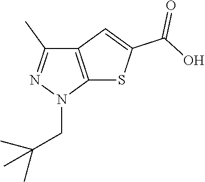 [SMILES: Cc1nn(CC(C)(C)C)c2sc(C(=O)O)cc12] |

### Synthetic Example 31

### Coupling Method A

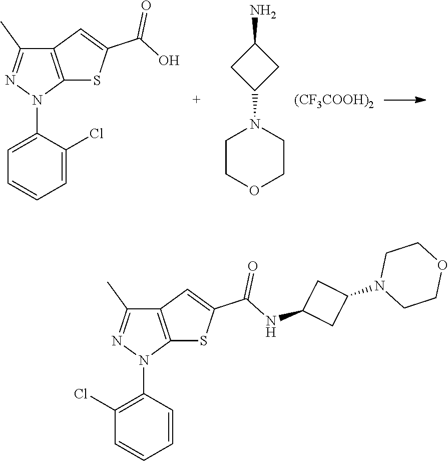

To a solution consisting of crude (1r,3r)-3-morpholinocyclobutan-1-amine bis(2,2,2-trifluoroacetate) (130 mg, 0.34 mmol) and DIPEA (0.15 mL, 0.85 mmol) in DMF (3 mL) was added 1-(2-chlorophenyl)-3-methyl-1H-thieno[2,3-c]pyrazole-5-carboxylic acid (50 mg, 0.17 mmol) followed by HBTU (65 mg, 0.17 mmol). The solution was stirred at RT for 48 hrs. The solution was concentrated and purified by amine column (50-100% Hep/EtOAc gradient) to yield 1-(2-chlorophenyl)-3-methyl-N-((1r,3r)-3-morpholinocyclobutyl)-1H-thieno[2,3-c]pyrazole-5-carboxamide (33 mg, 45%) as a white solid.

### Synthetic Example 32

### Coupling Method B

### Step 1: Synthesis of 1-(4,4-difluorocyclohexyl)-3-methyl-1H-thieno[2,3-c]pyrazole-5-carbonyl chloride

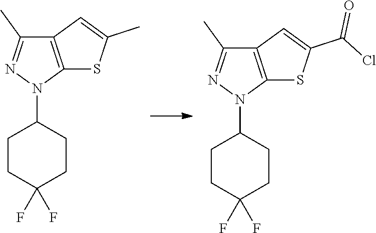

To a suspension of 1-(4,4-difluorocyclohexyl)-3-methyl-1H-thieno[2,3-c]pyrazole-5-carboxylic acid (150 mg, 0.5 mmol) in DCE (5 mL) was added SOCl₂ (3 mL, 40 mmol). The resulting slurry was heated at 70° C. for 4 hrs. After heating, the clear homogenous solution was concentrated under vacuum to yield 1-(4,4-difluorocyclohexyl)-3-methyl-1H-thieno[2,3-c]pyrazole-5-carbonyl chloride (159 mg, 100%) as an off-white solid. The crude solid was used in the next reaction without further purification.

### Step 2: Synthesis of 1-(4,4-difluorocyclohexyl)-N-((1r,3r)-3-(3,3-difluoropyrrolidin-1-yl)cyclobutyl)-3-methyl-1H-thieno[2,3-c]pyrazole-5-carboxamide

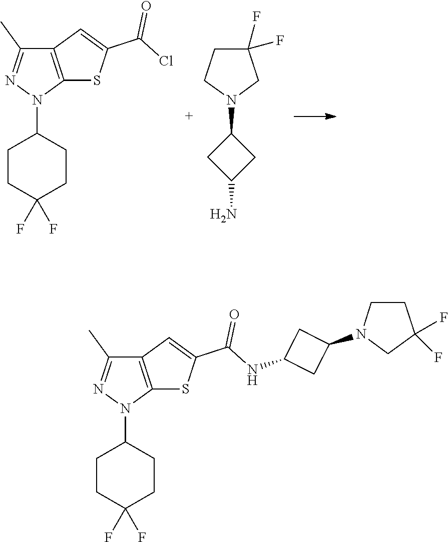

A solution consisting of 1-(4,4-difluorocyclohexyl)-3-methyl-1H-thieno[2,3-c]pyrazole-5-carbonyl chloride (159 mg, 0.5 mmol), 3-(3,3-difluoropyrrolidin-1-yl)cyclobutan-1-amine (114 mg, 0.65 mmol) and DIPEA (434 μL, 2.5 mmol) in THF (20 mL) was heated at 70° C. for 4 hrs. After evaporating the mixture to dryness, the residue was dissolved in DCM, washed with H2O and the organic portion dried over Na₂SO₄. The solvent was concentrated under vacuum to afford crude material that was purified using an amine column (50-100% Hep/EtOAc gradient) to yield the trans isomer 1-(4,4-difluorocyclohexyl)-N-((1r,3r)-3-(3,3-difluoropyrrolidin-1-yl)cyclobutyl)-3-methyl-1H-thieno[2,3-c]pyrazole-5-carboxamide (63 mg, 28%) as a white solid.

### Synthetic Example 33

### Synthesis of Compounds of Structures (I) and (II)

The following analogs were synthesized using either Coupling Method A or B with the appropriate carboxylic acid and amine precursors:
3-methyl-N-((1r,4r)-4-morpholinocyclohexyl)-1-phenyl-1H-thieno[2,3-c]pyrazole-5-carboxamide
N-((1r,4r)-4-(3,3-difluoroazetidin-1-yl)cyclohexyl)-1-isobutyl-3-methyl-1H-thieno[2,3-c]pyrazole-5-carboxamide
1-isobutyl-N-((1r,4r)-4-(4-isopropylpiperazin-1-yl)cyclohexyl)-3-methyl-1H-thieno[2,3-c]pyrazole-5-carboxamide
N-((1r,4r)-4-(4-cyclopropylpiperazin-1-yl)cyclohexyl)-1-isobutyl-3-methyl-1H-thieno[2,3-c]pyrazole-5-carboxamide
1-isobutyl-3-methyl-N-((1r,4r)-4-(4-(2,2,2-trifluoroethyl)piperazin-1-yl)cyclohexyl)-1H-thieno[2,3-c]pyrazole-5-carboxamide
N-((1r,4r)-4-(4-(2,2-difluoroethyl)piperazin-1-yl)cyclohexyl)-1-isobutyl-3-methyl-1H-thieno[2,3-c]pyrazole-5-carboxamide
1-((2r,6s)-2,6-dimethyltetrahydro-2H-pyran-4-yl)-N-((1r,4r)-4-(4-isopropylpiperazin-1-yl)cyclohexyl)-3-methyl-1H-thieno[2,3-c]pyrazole-5-carboxamide
N-((1r,4r)-4-(4-acetylpiperazin-1-yl)cyclohexyl)-1-((2r,6s)-2,6-dimethyltetrahydro-2H-pyran-4-yl)-3-methyl-1H-thieno[2,3-c]pyrazole-5-carboxamide
N-((1r,4r)-4-(4-acetylpiperazin-1-yl)cyclohexyl)-1-(4,4-difluorocyclohexyl)-3-methyl-1H-thieno[2,3-c]pyrazole-5-carboxamide
1-(4,4-difluorocyclohexyl)-N-((1r,4r)-4-(4-isopropylpiperazin-1-yl)cyclohexyl)-3-methyl-1H-thieno[2,3-c]pyrazole-5-carboxamide
1-(4,4-difluorocyclohexyl)-N-((1r,4r)-4-(4-(2,2-difluoroethyl)piperazin-1-yl)cyclohexyl)-3-methyl-1H-thieno[2,3-c]pyrazole-5-carboxamide
1-(4,4-difluorocyclohexyl)-N-((1r,4r)-4-(4-(2,2-trifluoroethyl)piperazin-1-yl)cyclohexyl)-3-methyl-1H-thieno[2,3-c]pyrazole-5-carboxamide
1-(4,4-difluorocyclohexyl)-3-methyl-N-((1r,4r)-4-morpholinocyclohexyl)-1H-thieno[2,3-c]pyrazole-5-carboxamide
1-(4,4-difluorocyclohexyl)-3-methyl-N-((1r,4r)-4-morpholinocyclohexyl)-1H-thieno[2,3-c]pyrazole-5-carboxamide
N-((1r,4r)-4-(4-cyclopropylpiperazin-1-yl)cyclohexyl)-1-(4,4-difluorocyclohexyl)-3-methyl-1H-thieno[2,3-c]pyrazole-5-carboxamide
3-methyl-N-((1r,4r)-4-morpholinocyclohexyl)-1-(pyridin-2-yl)-1H-thieno[2,3-c]pyrazole-5-carboxamide
N-((1r,4r)-4-(4-acetylpiperazin-1-yl)cyclohexyl)-1-isobutyl-3-methyl-1H-thieno[2,3-c]pyrazole-5-carboxamide
N-((1r,4r)-4-(3,3-difluoroazetidin-1-yl)cyclohexyl)-1-(4,4-difluorocyclohexyl)-3-methyl-1H-thieno[2,3-c]pyrazole-5-carboxamide
1-(2-chlorophenyl)-3-methyl-N-((1r,4r)-4-morpholinocyclohexyl)-1H-thieno[2,3-c]pyrazole-5-carboxamide
1-(4,4-difluorocyclohexyl)-3-methyl-N-((1r,3r)-3-morpholinocyclobutyl)-1H-thieno[2,3-c]pyrazole-5-carboxamide
N-((1r,4r)-4-(4-(dimethylcarbamoyl)piperazin-1-yl)cyclohexyl)-1-isobutyl-3-methyl-1H-thieno[2,3-c]pyrazole-5-carboxamide
1-(2-chlorophenyl)-3-methyl-N-((1r,3r)-3-morpholinocyclobutyl)-1H-thieno[2,3-c]pyrazole-5-carboxamide
N-((1r,4r)-4-(4-acetylpiperazin-1-yl)cyclohexyl)-1-(2-chlorophenyl)-3-methyl-1H-thieno[2,3-c]pyrazole-5-carboxamide
1-(cyclobutylmethyl)-N-((1r,4r)-4-(4-cyclopropylpiperazin-1-yl)cyclohexyl)-3-methyl-1H-thieno[2,3-c]pyrazole-5-carboxamide
1-(cyclobutylmethyl)-3-methyl-N-((1r,4r)-4-(4-(2,2,2-trifluoroethyl)piperazin-1-yl)cyclohexyl)-1H-thieno[2,3-c]pyrazole-5-carboxamide
1-(cyclobutylmethyl)-N-((1r,4r)-4-(3,3-difluoroazetidin-1-yl)cyclohexyl)-3-methyl-1H-thieno[2,3-c]pyrazole-5-carboxamide
N-((1r,4r)-4-(4-ethanethioylpiperazin-1-yl)cyclohexyl)-1-isobutyl-3-methyl-1H-thieno[2,3-c]pyrazole-5-carboxamide
N-((1r,4r)-4-(4-cyclopropylpiperazin-1-yl)cyclohexyl)-3-methyl-1-neopentyl-1H-thieno[2,3-c]pyrazole-5-carboxamide
3-methyl-N-((1r,4r)-3-morpholinocyclobutyl)-1-neopentyl-1H-thieno[2,3-c]pyrazole-5-carboxamide
1-(4,4-difluorocyclohexyl)-N-((1r,4r)-4-(3,3-difluoropyrrolidin-1-yl)cyclohexyl)-3-methyl-1H-thieno[2,3-c]pyrazole-5-carboxamide
N-((1r,4r)-4-(3,3-difluoropyrrolidin-1-yl)cyclohexyl)-3-methyl-1-neopentyl-1H-thieno[2,3-c]pyrazole-5-carboxamide
N-((1r,4r)-4-(4-(2,2-difluoroethyl)piperazin-1-yl)cyclohexyl)-3-methyl-1-neopentyl-1H-thieno[2,3-c]pyrazole-5-carboxamide
N-((1r,3r)-3-(4-acetylpiperazin-1-yl)cyclobutyl)-1-(2-chlorophenyl)-3-methyl-1H-thieno[2,3-c]pyrazole-5-carboxamide
N-((1r,4r)-3-(4-acetylpiperazin-1-yl)cyclobutyl)-1-(4,4-difluorocyclohexyl)-3-methyl-1H-thieno[2,3-c]pyrazole-5-carboxamide
N-((1r,4r)-4-(3,3-difluoroazetidin-1-yl)cyclohexyl)-3-methyl-1-neopentyl-1H-thieno[2,3-c]pyrazole-5-carboxamide
N-((1r,4r)-4-(4-isopropylpiperazin-1-yl)cyclohexyl)-3-methyl-1-neopentyl-1H-thieno[2,3-c]pyrazole-5-carboxamide
3-methyl-N-((1r,4r)-4-morpholinocyclohexyl)-1-neopentyl-1H-thieno[2,3-c]pyrazole-5-carboxamide
N-((1r,3r)-3-(4-cyclopropylpiperazin-1-yl)cyclobutyl)-1-(4,4-difluorocyclohexyl)-3-methyl-1H-thieno[2,3-c]pyrazole-5-carboxamide, Trifluoroacetate salt (Final compound was obtained after purification by prep HPLC)
N-((1r,3r)-3-(4-cyclopropylpiperazin-1-yl)cyclobutyl)-3-methyl-1-neopentyl-1H-thieno[2,3-c]pyrazole-5-carboxamide, Trifluoroacetate salt (Final compound was obtained after purification by prep HPLC)
1-(2-chlorophenyl)-N-((1r,3r)-3-(4-cyclopropylpiperazin-1-yl)cyclobutyl)-3-methyl-1H-thieno[2,3-c]pyrazole-5-carboxamide
3-methyl-N-(6-morpholinopyridin-3-yl)-1-neopentyl-1H-thieno[2,3-c]pyrazole-5-carboxamide
N-((1r,4r)-4-(3-cyanoazetidin-1-yl)cyclohexyl)-3-methyl-1-neopentyl-1H-thieno[2,3-c]pyrazole-5-carboxamide
N-((1r,4r)-4-(4-acetylpiperazin-1-yl)cyclohexyl)-3-methyl-1-neopentyl-1H-thieno[2,3-c]pyrazole-5-carboxamide, Trifluoroacetate salt (Final compound was obtained after purification by prep HPLC)
N-(5-chloro-6-morpholinopyridin-3-yl)-3-methyl-1-neopentyl-1H-thieno[2,3-c]pyrazole-5-carboxamide
1-(4,4-difluorocyclohexyl)-N-((1s,3s)-3-(3,3-difluoropyrrolidin-1-yl)cyclobutyl)-3-methyl-1H-thieno[2,3-c]pyrazole-5-carboxamide
1-(4,4-difluorocyclohexyl)-N-(3-(3,3-difluoropyrrolidin-1-yl)cyclobutyl)-3-methyl-1H-thieno[2,3-c]pyrazole-5-carboxamide
N-(3-cyano-4-morpholinophenyl)-3-methyl-1-neopentyl-1H-thieno[2,3-c]pyrazole-5-carboxamide
1-(4,4-difluorocyclohexyl)-N-((1r,4r)-4-(3-(difluoromethoxy)azetidin-1-yl)cyclohexyl)-3-methyl-1H-thieno[2,3-c]pyrazole-5-carboxamide
N-((1r,4r)-4-(3-(difluoromethoxy)azetidin-1-yl)cyclohexyl)-3-methyl-1-neopentyl-1H-thieno[2,3-c]pyrazole-5-carboxamide
1-(2-chlorophenyl)-N-((1r,4r)-4-(3-(difluoromethoxy)azetidin-1-yl)cyclohexyl)-3-methyl-1H-thieno[2,3-c]pyrazole-5-carboxamide
N-((1r,4r)-4-(3-cyanoazetidin-1-yl)cyclohexyl)-1-isobutyl-3-methyl-1H-thieno[2,3-c]pyrazole-5-carboxamide
N-((1r,4r)-4-(3-(difluoromethoxy)azetidin-1-yl)cyclohexyl)-1-isobutyl-3-methyl-1H-thieno[2,3-c]pyrazole-5-carboxamide
N-((1r,4r)-4-(3-methoxyazetidin-1-yl)cyclohexyl)-3-methyl-1-neopentyl-1H-thieno[2,3-c]pyrazole-5-carboxamide
1-isobutyl-N-((1r,4r)-4-(3-methoxyazetidin-1-yl)cyclohexyl)-3-methyl-1H-thieno[2,3-c]pyrazole-5-carboxamide

### Biological Example 1

### Example Description

PDE7A and PDE7B Assay. Compounds were tested for their activity by measuring the inhibition of PDE7A or PDE7B hydrolysis of [3H]cAMP to [3H]AMP. Generally, eight dilutions of compound were assayed in 50 mM Tris-HCl pH 7.5, 8.3 mM MgCl₂, 0.5 mg/mL BSA, 1.7 mM EGTA, 16 nM [3H]cAMP, and 1% DMSO. PDE7A or PDE7B (typically 1-5 ng/mL) (BPS BioSciences, CA) is added to initiate the reaction. The reaction was incubated at 30° C. for 20 minutes. PDE7A and PDE7B hydrolysis was terminated by the addition of yttrium silicate beads (GE Healthcare, RPNQ0150) and counted on a Wallac Microbeta scintillation counter, 1-2 hours following the addition of the beads. Data was analyzed using XLfit (Microsoft) from which ICso values were obtained.

TABLE 3
Activity of Representative Compounds of Structure (I)

| | PDE 7A | PDE 7B | | PDE 7A | PDE 7B |
|---|---|---|---|---|---|
| Compound | IC₅₀ | IC₅₀ | Compound | IC₅₀ | IC₅₀ |
| No. | (nM) | (nM) | No. | (nM) | (nM) |
| I-1 | ++ | ++ | I-2 | ++++ | ++++ |
| I-3 | ++++ | +++ | I-4 | +++++ | +++++ |
| I-5 | +++ | +++ | I-6 | +++ | +++ |
| I-7 | ++++ | ++++ | I-8 | ++ | ++ |

TABLE 4
Activity of Representative Compounds of Structure (II)

| | PDE 7A | PDE 7B | | PDE 7A | PDE 7B |
|---|---|---|---|---|---|
| Compound | IC₅₀ | IC₅₀ | Compound | IC₅₀ | IC₅₀ |
| No. | (nM) | (nM) | No. | (nM) | (nM) |
| II-1 | ++ | + | II-2 | +++ | ++ |
| II-3 | ++++ | ++++ | II-4 | ++++ | +++ |
| II-5 | +++ | ++++ | II-6 | ++++ | +++ |
| II-7 | +++ | +++ | II-8 | +++ | +++ |
| II-9 | +++++ | +++++ | II-10 | +++++ | +++++ |
| II-11 | ++++ | ++++ | II-12 | +++++ | ++++ |
| II-13 | ++++ | ++++ | II-14 | +++ | +++ |
| II-15 | ++++ | ++++ | II-16 | ++++ | +++ |
| II-17 | +++++ | +++++ | II-18 | ++++ | ++++ |
| II-19 | ++++ | +++ | II-20 | +++ | ++ |
| II-21 | ++++ | +++ | II-22 | +++ | +++ |
| II-23 | ++++ | +++ | II-24 | +++++ | +++++ |
| II-25 | ++++ | +++ | II-26 | +++++ | +++++ |
| II-27 | +++++ | +++++ | II-28 | ++++ | ++++ |
| II-29 | ++++ | ++++ | II-30 | +++++ | +++++ |
| II-31 | +++++ | +++++ | II-32 | +++++ | +++++ |
| II-33 | ++++ | ++++ | II-34 | ++++ | +++ |
| II-35 | ++++ | ++++ | II-36 | +++++ | +++++ |
| II-37 | +++++ | +++++ | II-38 | ++ | + |
| II-39 | +++ | +++ | II-40 | ++ | ++ |
| II-41 | ++++ | +++++ | II-42 | +++++ | +++++ |
| II-43 | ++++ | ++++ | II-44 | +++ | ++ |
| II-45 | +++++ | ++++ | II-46 | − | − |

IC₅₀ activity in Tables 3 and 4:
+ IC₅₀ from 500 to less than 2500 nM
++ IC₅₀ ranges from 100 to less than 500 nM
+++ IC₅₀ ranges from 30 to less than 100 nM
++++ IC₅₀ ranges from 10 to less than 30 nM
+++++ IC₅₀ less than 10 nM
− denotes a value was not determined

The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications, and publications to provide yet further embodiments.

These and other changes can be made to the embodiments considering the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A compound of Structure (I): 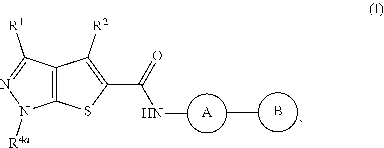
wherein:
ring A is optionally substituted C₃-C₆ cycloalkyl;
ring B is optionally substituted C₄-C₈ cycloalkyl, optionally substituted C₆-C₁₀ aryl, or optionally substituted 4-10 membered heterocyclyl;
R¹ is hydrogen or optionally substituted C₁₋₃ alkyl;
R² is hydrogen, optionally substituted C₁₋₃ alkyl, or halo;
R^{4a} is optionally substituted phenyl or optionally substituted 5-10 membered heteroaryl; provided that:
when R^{4a} is unsubstituted phenyl, then ring A is C₅-C₆ cycloalkyl and ring B optionally substituted C₄-C₈ cycloalkyl, optionally substituted C₆-C₁₀ aryl, or optionally substituted 6-10 membered heterocyclyl,
as a stereoisomer, enantiomer, or tautomer thereof or a mixture thereof; or a pharmaceutically acceptable salt, solvate, or prodrug thereof.

2. The compound of claim 1, wherein R¹ is unsubstituted C₁₋₃ alkyl
as a stereoisomer, enantiomer, or tautomer thereof or a mixture thereof; or a pharmaceutically acceptable salt, solvate, or prodrug thereof.

3. The compound of claim 1, wherein R¹ is methyl
as a stereoisomer, enantiomer, or tautomer thereof or a mixture thereof; or a pharmaceutically acceptable salt, solvate, or prodrug thereof.

4. The compound of claim 1, wherein R² is hydrogen
as a stereoisomer, enantiomer, or tautomer thereof or a mixture thereof; or a pharmaceutically acceptable salt, solvate, or prodrug thereof.

5. The compound of claim 1, wherein R² is unsubstituted C₁₋₃ alkyl
as a stereoisomer, enantiomer, or tautomer thereof or a mixture thereof; or a pharmaceutically acceptable salt, solvate, or prodrug thereof.

6. The compound of claim 1, wherein R² is halo
as a stereoisomer, enantiomer, or tautomer thereof or a mixture thereof; or a pharmaceutically acceptable salt, solvate, or prodrug thereof.

7. The compound of claim 6, wherein R² is chloro, fluoro, or bromo
as a stereoisomer, enantiomer, or tautomer thereof or a mixture thereof; or a pharmaceutically acceptable salt, solvate, or prodrug thereof.

8. The compound of claim 6, wherein R² is fluoro
as a stereoisomer, enantiomer, or tautomer thereof or a mixture thereof; or a pharmaceutically acceptable salt, solvate, or prodrug thereof.

9. The compound of claim 1, wherein the compound has one of the following Structures (IB) or (IC): 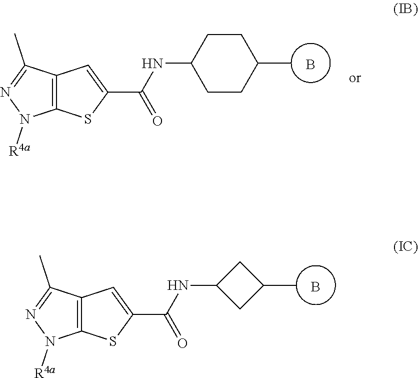
as a stereoisomer, enantiomer, or tautomer thereof or a mixture thereof; or a pharmaceutically acceptable salt, solvate, or prodrug thereof.

10. The compound of claim 1, wherein ring B is optionally substituted 4-10 membered heterocyclyl, as a stereoisomer, enantiomer, or tautomer thereof or a mixture thereof; or a pharmaceutically acceptable salt, solvate, or prodrug thereof.

11. The compound of claim 1, wherein ring B has one of the following structures: 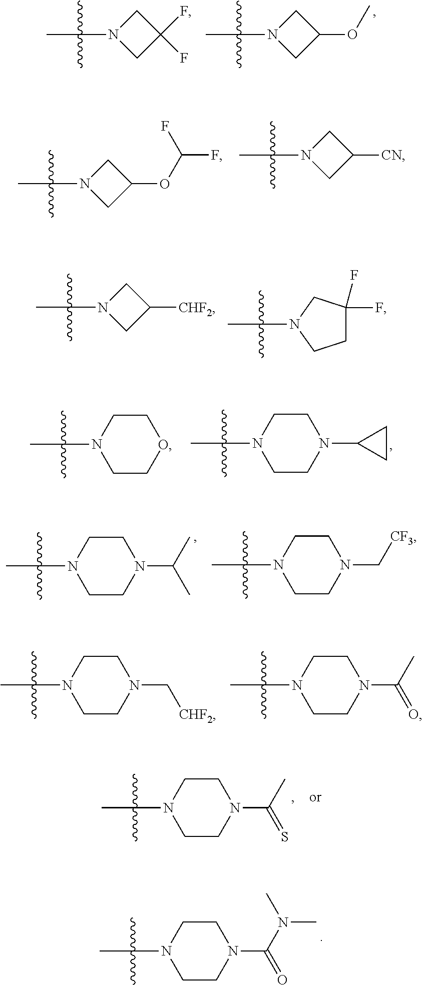
as a stereoisomer, enantiomer, or tautomer thereof or a mixture thereof; or a pharmaceutically acceptable salt, solvate, or prodrug thereof.

12. The compound of claim 1, wherein ring B has one of the following structures: 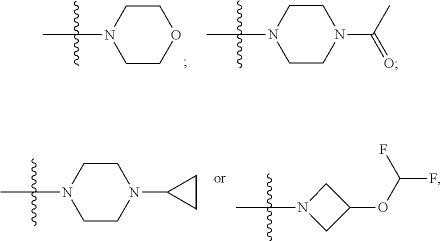
as a stereoisomer, enantiomer, or tautomer thereof or a mixture thereof; or a pharmaceutically acceptable salt, solvate, or prodrug thereof.

13. The compound of claim 1, wherein R^{4a} has one of the following structures: 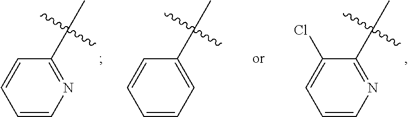 [SMILES: *c1ccccn1 ; *c1ccccc1 ; *c1ncccc1Cl]
as a stereoisomer, enantiomer, or tautomer thereof or a mixture thereof; or a pharmaceutically acceptable salt, solvate, or prodrug thereof.

14. The compound of claim 1, wherein R^{4a} has the following structure: 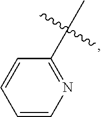 [SMILES: *c1ccccn1]
as a stereoisomer, enantiomer, or tautomer thereof or a mixture thereof; or a pharmaceutically acceptable salt, solvate, or prodrug thereof.

15. The compound of claim 1, wherein R^{4a} has the following structure: 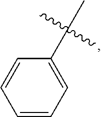 [SMILES: *c1ccccc1]
as a stereoisomer, enantiomer, or tautomer thereof or a mixture thereof; or a pharmaceutically acceptable salt, solvate, or prodrug thereof.

16. The compound of claim 1, wherein R^{4a} has the following structure: 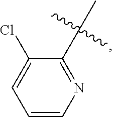 [SMILES: *c1ncccc1Cl]
as a stereoisomer, enantiomer, or tautomer thereof or a mixture thereof; or a pharmaceutically acceptable salt, solvate, or prodrug thereof.

17. A compound of Structure (II): 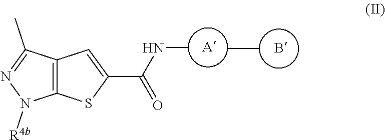
wherein:
ring A′ is optionally substituted C₃-C₈ cycloalkyl, or has one of the following structures: 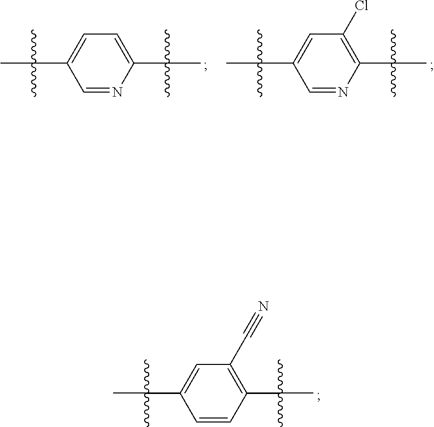 [SMILES: *c1ccc(*)nc1 ; *c1cnc(*)c(Cl)c1 ; *c1ccc(*)c(C#N)c1]
ring B′ is optionally substituted 4-10 membered heterocyclyl; and
R^{4b} is —CH₂CH(CH₃)₂, —CH₂C(CH₃)₃, 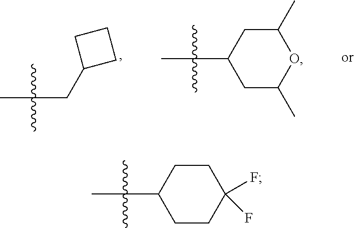
provided that:
when R^{4b} is —CH₂CH(CH₃)₂ and ring A′ has the following structure: 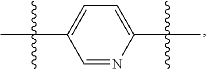 [SMILES: *c1ccc(*)nc1]
then ring B′ is not piperidinyl or 2,6-dimethyl-4-morpholinyl,
as a stereoisomer, enantiomer, or tautomer thereof or a mixture thereof; or a pharmaceutically acceptable salt, solvate, or prodrug thereof.

18. The compound of claim 17, wherein R^{4b} is —CH₂CH(CH₃)₂ or —CH₂C(CH₃)₃, as a stereoisomer, enantiomer, or tautomer thereof or a mixture thereof; or a pharmaceutically acceptable salt, solvate, or prodrug thereof.

19. The compound of claim 17, wherein R^{4b} has one of the following structures: 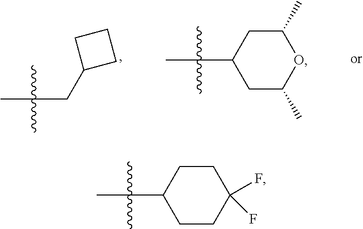
as a stereoisomer, enantiomer, or tautomer thereof or a mixture thereof; or a pharmaceutically acceptable salt, solvate, or prodrug thereof.

20. The compound of claim 17, wherein R^{4b} has the following structure: 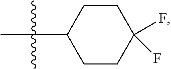 [SMILES: *C1CCC(F)(F)CC1]
as a stereoisomer, enantiomer, or tautomer thereof or a mixture thereof; or a pharmaceutically acceptable salt, solvate, or prodrug thereof.

21. The compound of claim 17, wherein ring A′ has one of the following structures: 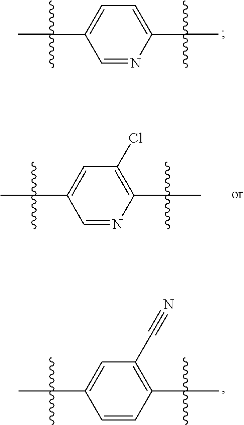 [SMILES: *c1ccc(*)nc1 ; *c1cnc(*)c(Cl)c1 ; *c1ccc(*)c(C#N)c1]
as a stereoisomer, enantiomer, or tautomer thereof or a mixture thereof; or a pharmaceutically acceptable salt, solvate, or prodrug thereof.

22. The compound of claim 17, wherein the compound has one of the following Structures (JIB) or (IIC): 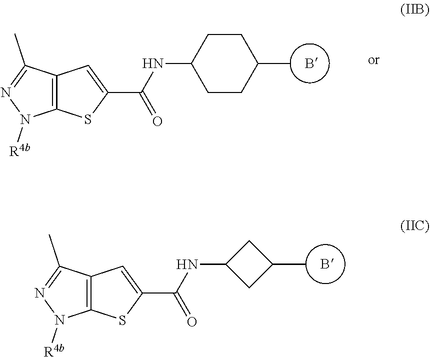
as a stereoisomer, enantiomer, or tautomer thereof or a mixture thereof; or a pharmaceutically acceptable salt, solvate, or prodrug thereof.

23. The compound of claim 17, wherein ring B′ is optionally substituted with one or more substituents selected from the group consisting of C₁-C₆ alkyl, halo, C₁-C₆ haloalkyl, cyano, C₁-C₃ alkoxy, C₁-C₃ haloalkoxy, —C(═O)alkyl, C(═S)alkyl, C₃-C₆ cycloalkyl, and C(═O)NR^{b1}R^{b2},
wherein:
R^{b1} and R^{b2} are each independently hydrogen or C₁-C₃ alkyl,
as a stereoisomer, enantiomer, or tautomer thereof or a mixture thereof; or a pharmaceutically acceptable salt, solvate, or prodrug thereof.

24. The compound of claim 17, wherein ring B′ is optionally substituted with one or more substituents selected from the group consisting of —CH(CH₃)₂, —C(═O)CH₃, fluoro, cyclopropyl, —CH₂CF₃, —CH₂CHF₂, C(═O)N(CH₃)₂, —C(═S)CH₃, cyano, —OCHF₂, and —OCH₃,
as a stereoisomer, enantiomer, or tautomer thereof or a mixture thereof; or a pharmaceutically acceptable salt, solvate, or prodrug thereof.

25. The compound of claim 17, wherein ring B′ has one of the following structures: 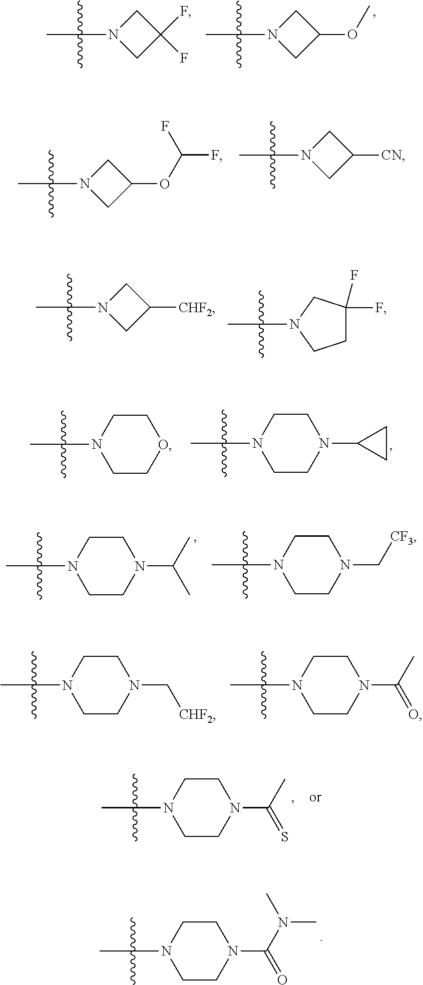
as a stereoisomer, enantiomer, or tautomer thereof or a mixture thereof; or a pharmaceutically acceptable salt, solvate, or prodrug thereof.

26. The compound of claim 17, wherein ring B′ has one of the following structures: 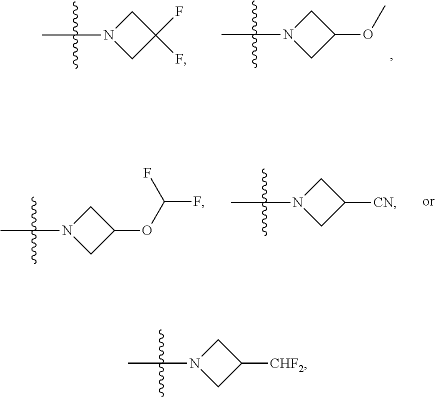
as a stereoisomer, enantiomer, or tautomer thereof or a mixture thereof; or a pharmaceutically acceptable salt, solvate, or prodrug thereof.

27. The compound of claim 17, wherein ring B′ has one of the following structures: 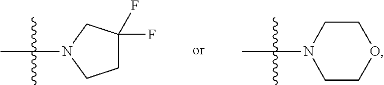 [SMILES: *N1CCC(F)(F)C1 ; "or" ; *N1CCOCC1]
as a stereoisomer, enantiomer, or tautomer thereof or a mixture thereof; or a pharmaceutically acceptable salt, solvate, or prodrug thereof.

28. The compound of claim 17, wherein ring B′ has one of the following structures: 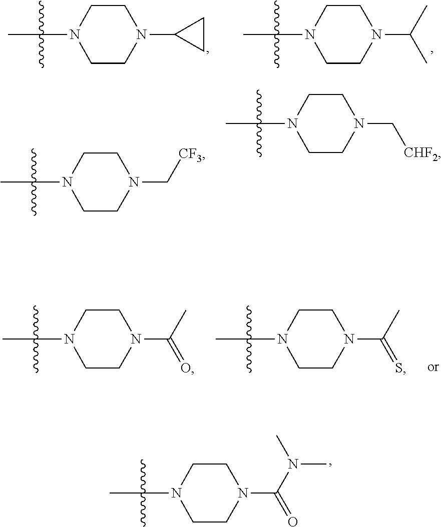
as a stereoisomer, enantiomer, or tautomer thereof or a mixture thereof; or a pharmaceutically acceptable salt, solvate, or prodrug thereof.

29. The compound of claim 17, wherein 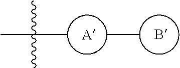
has one of the following structures: 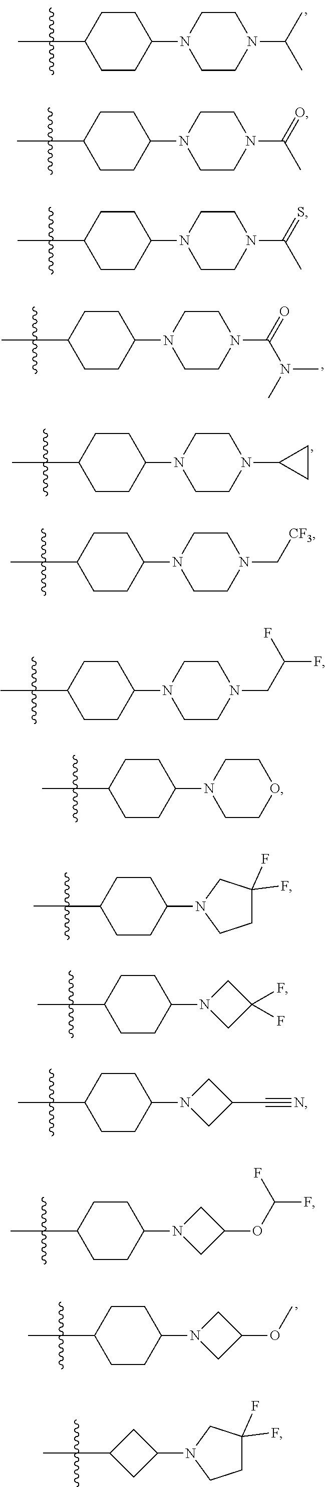 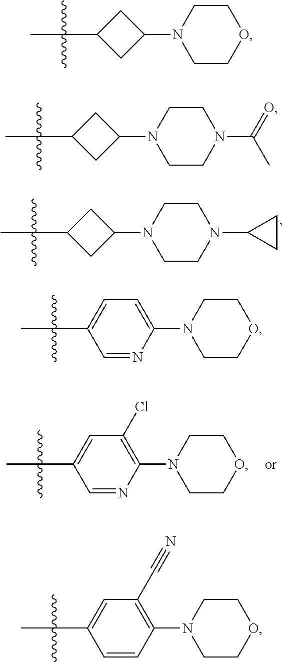
as a stereoisomer, enantiomer, or tautomer thereof or a mixture thereof; or a pharmaceutically acceptable salt, solvate, or prodrug thereof.

30. A compound selected from:
3-methyl-N-((1r,4r)-4-morpholinocyclohexyl)-1-(pyridin-2-yl)-1H-thieno[2,3-c]pyrazole-5-carboxamide;
1-(2-chlorophenyl)-3-methyl-N-((1r,4r)-4-morpholinocyclohexyl)-1H-thieno[2,3-c]pyrazole-5-carboxamide;
1-(2-chlorophenyl)-3-methyl-N-((1r,3r)-3-morpholinocyclobutyl)-1H-thieno[2,3-c]pyrazole-5-carboxamide;
N-((1r,4r)-4-(4-acetylpiperazin-1-yl)cyclohexyl)-1-(2-chlorophenyl)-3-methyl-1H-thieno[2,3-c]pyrazole-5-carboxamide;
N-((1r,3r)-3-(4-acetylpiperazin-1-yl)cyclobutyl)-1-(2-chlorophenyl)-3-methyl-1H-thieno[2,3-c]pyrazole-5-carboxamide;
1-(2-chlorophenyl)-N-((1r,3r)-3-(4-cyclopropylpiperazin-1-yl)cyclobutyl)-3-methyl-1H-thieno[2,3-c]pyrazole-5-carboxamide;
1-(2-chlorophenyl)-3-methyl-N-(6-morpholinopyridin-3-yl)-1H-thieno[2,3-c]pyrazole-5-carboxamide;
1-(2-chlorophenyl)-N-((1r,4r)-4-(3-(difluoromethoxy)azetidin-1-yl)cyclohexyl)-3-methyl-1H-thieno[2,3-c]pyrazole-5-carboxamide;
1-((2r,6s)-2,6-dimethyltetrahydro-2H-pyran-4-yl)-N-((1r,4r)-4-(4-isopropylpiperazin-1-yl)cyclohexyl)-3-methyl-1H-thieno[2,3-c]pyrazole-5-carboxamide;
N-((1r,4r)-4-(4-acetylpiperazin-1-yl)cyclohexyl)-1-((2r,6s)-2,6-dimethyltetrahydro-2H-pyran-4-yl)-3-methyl-1H-thieno[2,3-c]pyrazole-5-carboxamide;
1-(4,4-difluorocyclohexyl)-N-(3-(3,3-difluoropyrrolidin-1-yl)cyclobutyl)-3-methyl-1H-thieno[2,3-c]pyrazole-5-carboxamide;
N-((1r,4r)-4-(3,3-difluoroazetidin-1-yl)cyclohexyl)-1-isobutyl-3-methyl-1H-thieno[2,3-c]pyrazole-5-carboxamide;
1-isobutyl-N-((1r,4r)-4-(4-isopropylpiperazin-1-yl)cyclohexyl)-3-methyl-1H-thieno[2,3-c]pyrazole-5-carboxamide;
N-((1r,4r)-4-(4-cyclopropylpiperazin-1-yl)cyclohexyl)-1-isobutyl-3-methyl-1H-thieno[2,3-c]pyrazole-5-carboxamide;
1-isobutyl-3-methyl-N-((1r,4r)-4-(4-(2,2,2-trifluoroethyl)piperazin-1-yl)cyclohexyl)-1H-thieno[2,3-c]pyrazole-5-carboxamide;
N-((1r,4r)-4-(4-(2,2-difluoroethyl)piperazin-1-yl)cyclohexyl)-1-isobutyl-3-methyl-1H-thieno[2,3-c]pyrazole-5-carboxamide;
N-((1r,4r)-4-(4-acetylpiperazin-1-yl)cyclohexyl)-1-(4,4-difluorocyclohexyl)-3-methyl-1H-thieno[2,3-c]pyrazole-5-carboxamide;
1-(4,4-difluorocyclohexyl)-N-((1r,4r)-4-(4-isopropylpiperazin-1-yl)cyclohexyl)-3-methyl-1H-thieno[2,3-c]pyrazole-5-carboxamide;
1-(4,4-difluorocyclohexyl)-N-((1r,4r)-4-(4-(2,2-difluoroethyl)piperazin-1-yl)cyclohexyl)-3-methyl-1H-thieno[2,3-c]pyrazole-5-carboxamide;
1-(4,4-difluorocyclohexyl)-N-((1r,4r)-4-(4-(2,2-trifluoroethyl)piperazin-1-yl)cyclohexyl)-3-methyl-1H-thieno[2,3-c]pyrazole-5-carboxamide;
1-(4,4-difluorocyclohexyl)-3-methyl-N-((1r,4r)-4-morpholinocyclohexyl)-1H-thieno[2,3-c]pyrazole-5-carboxamide;
1-(4,4-difluorocyclohexyl)-3-methyl-N-((1r,4r)-4-morpholinocyclohexyl)-1H-thieno[2,3-c]pyrazole-5-carboxamide;
N-((1r,4r)-4-(4-cyclopropylpiperazin-1-yl)cyclohexyl)-1-(4,4-difluorocyclohexyl)-3-methyl-1H-thieno[2,3-c]pyrazole-5-carboxamide;
N-((1r,4r)-4-(4-acetylpiperazin-1-yl)cyclohexyl)-1-isobutyl-3-methyl-1H-thieno[2,3-c]pyrazole-5-carboxamide;
N-((1r,4r)-4-(3,3-difluoroazetidin-1-yl)cyclohexyl)-1-(4,4-difluorocyclohexyl)-3-methyl-1H-thieno[2,3-c]pyrazole-5-carboxamide;
1-(4,4-difluorocyclohexyl)-3-methyl-N-((1r,3r)-3-morpholinocyclobutyl)-1H-thieno[2,3-c]pyrazole-5-carboxamide;
N-((1r,4r)-4-(4-(dimethylcarbamoyl)piperazin-1-yl)cyclohexyl)-1-isobutyl-3-methyl-1H-thieno[2,3-c]pyrazole-5-carboxamide;
1-(cyclobutylmethyl)-N-((1r,4r)-4-(4-cyclopropylpiperazin-1-yl)cyclohexyl)-3-methyl-1H-thieno[2,3-c]pyrazole-5-carboxamide;
1-(cyclobutylmethyl)-3-methyl-N-((1r,4r)-4-(4-(2,2,2-trifluoroethyl)piperazin-1-yl)cyclohexyl)-1H-thieno[2,3-c]pyrazole-5-carboxamide;
1-(cyclobutylmethyl)-N-((1r,4r)-4-(3,3-difluoroazetidin-1-yl)cyclohexyl)-3-methyl-1H-thieno[2,3-c]pyrazole-5-carboxamide;
N-((1r,4r)-4-(4-ethanethioylpiperazin-1-yl)cyclohexyl)-1-isobutyl-3-methyl-1H-thieno[2,3-c]pyrazole-5-carboxamide;
N-((1r,4r)-4-(4-cyclopropylpiperazin-1-yl)cyclohexyl)-3-methyl-1-neopentyl-1H-thieno[2,3-c]pyrazole-5-carboxamide;
3-methyl-N-((1r,3r)-3-morpholinocyclobutyl)-1-neopentyl-1H-thieno[2,3-c]pyrazole-5-carboxamide;
1-(4,4-difluorocyclohexyl)-N-((1r,4r)-4-(3,3-difluoropyrrolidin-1-yl)cyclohexyl)-3-methyl-1H-thieno[2,3-c]pyrazole-5-carboxamide;
N-((1r,4r)-4-(3,3-difluoropyrrolidin-1-yl)cyclohexyl)-3-methyl-1-neopentyl-1H-thieno[2,3-c]pyrazole-5-carboxamide;
N-((1r,4r)-4-(4-(2,2-difluoroethyl)piperazin-1-yl)cyclohexyl)-3-methyl-1-neopentyl-1H-thieno[2,3-c]pyrazole-5-carboxamide;
N-((1r,3r)-3-(4-acetylpiperazin-1-yl)cyclobutyl)-1-(4,4-difluorocyclohexyl)-3-methyl-1H-thieno[2,3-c]pyrazole-5-carboxamide;
N-((1r,4r)-4-(3,3-difluoroazetidin-1-yl)cyclohexyl)-3-methyl-1-neopentyl-1H-thieno[2,3-c]pyrazole-5-carboxamide;
N-((1r,4r)-4-(4-isopropylpiperazin-1-yl)cyclohexyl)-3-methyl-1-neopentyl-1H-thieno[2,3-c]pyrazole-5-carboxamide;
3-methyl-N-((1r,4r)-4-morpholinocyclohexyl)-1-neopentyl-1H-thieno[2,3-c]pyrazole-5-carboxamide;
N-((1r,3r)-3-(4-cyclopropylpiperazin-1-yl)cyclobutyl)-1-(4,4-difluorocyclohexyl)-3-methyl-1H-thieno[2,3-c]pyrazole-5-carboxamide;
N-((1r,3r)-3-(4-cyclopropylpiperazin-1-yl)cyclobutyl)-3-methyl-1-neopentyl-1H-thieno[2,3-c]pyrazole-5-carboxamide;
3-methyl-N-(6-morpholinopyridin-3-yl)-1-neopentyl-1H-thieno[2,3-c]pyrazole-5-carboxamide;
N-((1r,4r)-4-(3-cyanoazetidin-1-yl)cyclohexyl)-3-methyl-1-neopentyl-1H-thieno[2,3-c]pyrazole-5-carboxamide;
N-((1r,4r)-4-(4-acetylpiperazin-1-yl)cyclohexyl)-3-methyl-1-neopentyl-1H-thieno[2,3-c]pyrazole-5-carboxamide;
N-(5-chloro-6-morpholinopyridin-3-yl)-3-methyl-1-neopentyl-1H-thieno[2,3-c]pyrazole-5-carboxamide;
1-(4,4-difluorocyclohexyl)-N-((1S,3S)-3-(3,3-difluoropyrrolidin-1-yl)cyclobutyl)-3-methyl-1H-thieno[2,3-c]pyrazole-5-carboxamide;
N-(3-cyano-4-morpholinophenyl)-3-methyl-1-neopentyl-1H-thieno[2,3-c]pyrazole-5-carboxamide;
1-(4,4-difluorocyclohexyl)-N-((1r,4r)-4-(3-(difluoromethoxy)azetidin-1-yl)cyclohexyl)-3-methyl-1H-thieno[2,3-c]pyrazole-5-carboxamide;
N-((1r,4r)-4-(3-(difluoromethoxy)azetidin-1-yl)cyclohexyl)-3-methyl-1-neopentyl-1H-thieno[2,3-c]pyrazole-5-carboxamide;
N-((1r,4r)-4-(3-cyanoazetidin-1-yl)cyclohexyl)-1-isobutyl-3-methyl-1H-thieno[2,3-c]pyrazole-5-carboxamide;
N-((1r,4r)-4-(3-(difluoromethoxy)azetidin-1-yl)cyclohexyl)-1-isobutyl-3-methyl-1H-thieno[2,3-c]pyrazole-5-carboxamide;
N-((1r,4r)-4-(3-methoxyazetidin-1-yl)cyclohexyl)-3-methyl-1-neopentyl-1H-thieno[2,3-c]pyrazole-5-carboxamide; and
1-isobutyl-N-((1r,4r)-4-(3-methoxyazetidin-1-yl)cyclohexyl)-3-methyl-1H-thieno[2,3-c]pyrazole-5-carboxamide,
as a stereoisomer, enantiomer, or tautomer thereof or a mixture thereof; or a pharmaceutically acceptable salt, solvate, or prodrug thereof.

31. The compound of claim 1, wherein the compound has a molecular weight of less than about 500 g/mole, as a stereoisomer, enantiomer, or tautomer thereof or a mixture thereof; or a pharmaceutically acceptable salt, solvate, or prodrug thereof.

32. The compound of claim 1, wherein the compound has a molecular weight of less than about 450 g/mole, as a stereoisomer, enantiomer, or tautomer thereof or a mixture thereof; or a pharmaceutically acceptable salt, solvate, or prodrug thereof.

33. The compound of claim 1, wherein the compound has an IC₅₀ for inhibiting PDE 7A and/or PDE 7B activity of less than about 1 μM, as a stereoisomer, enantiomer, or tautomer thereof or a mixture thereof; or a pharmaceutically acceptable salt, solvate, or prodrug thereof.

34. The compound of claim 1, wherein the compound has an IC₅₀ for inhibiting PDE 7A and/or PDE 7B activity of less than about 100 nM, as a stereoisomer, enantiomer, or tautomer thereof or a mixture thereof; or a pharmaceutically acceptable salt, solvate, or prodrug thereof.

35. The compound of claim 1, wherein the compound is a selective PDE 7 inhibitor for which the lesser of the IC₅₀ for inhibiting PDE 7A activity and the IC₅₀ for inhibiting PDE 7B activity is less than one-tenth the IC₅₀ that the compound has for inhibiting the activity of any other PDE enzyme from the PDE 1-6 and PDE 8-11 enzyme families, as a stereoisomer, enantiomer, or tautomer thereof or a mixture thereof; or a pharmaceutically acceptable salt, solvate, or prodrug thereof.

36. The compound of claim 1, wherein the compound is a highly selective PDE 7 inhibitor for which the lesser of the IC₅₀ for inhibiting PDE 7A activity and the IC₅₀ for inhibiting PDE 7B activity is less than one-fiftieth the IC₅₀ that the compound has for inhibiting the activity of any other PDE enzyme from the PDE 1-6 and PDE 8-11 enzyme families, as a stereoisomer, enantiomer, or tautomer thereof or a mixture thereof; or a pharmaceutically acceptable salt, solvate, or prodrug thereof.

37. A method of treating an addiction to an addictive agent, comprising administering to a subject in need thereof an amount of an inhibitor of a phosphodiesterase 7 (PDE 7) of claim 1 effective for the treatment of the addiction, as a stereoisomer, enantiomer, or tautomer thereof or a mixture thereof; or a pharmaceutically acceptable salt, solvate, or prodrug thereof.

38. The method of claim 37, wherein the subject is addicted to an addictive agent selected from the group consisting of alcohol, nicotine, marijuana, marijuana derivatives, opioid agonists, benzodiazepines, barbiturates, and psychostimulants.

39. The method of claim 38, wherein the addictive agent is alcohol or nicotine.

40. (canceled)

41. The method of claim 38, wherein the opioid agonist is selected from the group consisting of morphine, methadone, fentanyl, sufentanil, and heroin.

42. The method of claim 38, wherein the psychostimulant is cocaine, amphetamines, or amphetamine derivatives.

43. (canceled)

44. A method of treating a movement disorder, comprising administering to a subject in need thereof an amount of an inhibitor of a phosphodiesterase 7 (PDE 7) of claim 1 effective for the treatment of the movement disorder, as a stereoisomer, enantiomer, or tautomer thereof or a mixture thereof; or a pharmaceutically acceptable salt, solvate, or prodrug thereof.

45. The method of claim 44, wherein the movement disorder is a neurological condition that causes problems with movement.

46. The method of claim 44, wherein the movement disorder is a tremor, Tourette syndrome, dystonia, Parkinson's disease, Huntington's disease, multiple system atrophy (MSA), myoclonus, progressive supranuclear palsy, Rett syndrome, secondary parkinsonism, spasticity, or Wilson's disease.

47. A method for preparing a pyrazole compound, the method comprising contacting a hydrazine compound, or a salt thereof, having the following structure: 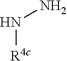
wherein:
R^{4c} is alkyl, aryl, arylalkyl, cycloalkyl, cycloalkylalkyl, heterocyclyl, heterocyclylalkyl, heteroaryl, or heteroarylalkyl;
with an ester compound (or a salt thereof) having the following structure: 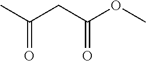
thereby forming the pyrazole compound, wherein the pyrazole compound has the following structure: 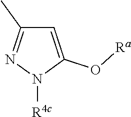
wherein:
R^{a} is hydrogen or —CH₃, as a stereoisomer, enantiomer, or tautomer thereof or a mixture thereof; or a pharmaceutically acceptable salt or solvate thereof.

48. The method of claim 47, wherein the hydrazine compound, the ester compound, and a polar aprotic solvent are combined to form a first mixture.

49. The method of claim 48, wherein the first mixture is not heated at a temperature above 30° C.

50. The method of claim 48, wherein
the first mixture is free of acetic acid.

51. The method of claim 47, wherein the method further comprises contacting the pyrazole compound with phosphorus oxychloride thereby converting the pyrazole compound to an aldehyde compound having the following structure: 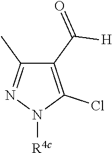
as a stereoisomer, enantiomer, or tautomer thereof or a mixture thereof; or a pharmaceutically acceptable salt or solvate thereof.

52. A method of treating an addiction to an addictive agent, comprising administering to a subject in need thereof an amount of an inhibitor of a phosphodiesterase 7 (PDE 7) of claim 17 effective for the treatment of the addiction, as a stereoisomer, enantiomer, or tautomer thereof or a mixture thereof; or a pharmaceutically acceptable salt, solvate, or prodrug thereof.

53. The method of claim 52, wherein the subject is addicted to an addictive agent selected from the group consisting of alcohol, nicotine, marijuana, marijuana derivatives, opioid agonists, benzodiazepines, barbiturates, and psychostimulants.

54. The method of claim 53, wherein the addictive agent is alcohol or nicotine.

55. The method of claim 53, wherein the addictive agent is selected from the group consisting of morphine, methadone, fentanyl, sufentanil, heroin, cocaine, amphetamines, and amphetamine derivatives.

56. A method of treating a movement disorder, comprising administering to a subject in need thereof an amount of an inhibitor of a phosphodiesterase 7 (PDE 7) of claim 17 effective for the treatment of the movement disorder, as a stereoisomer, enantiomer, or tautomer thereof or a mixture thereof; or a pharmaceutically acceptable salt, solvate, or prodrug thereof.

57. The method of claim 56, wherein the movement disorder is a neurological condition that causes problems with movement.

58. The method of claim 56, wherein the movement disorder is a tremor, Tourette syndrome, dystonia, Parkinson's disease, Huntington's disease, multiple system atrophy (MSA), myoclonus, progressive supranuclear palsy, Rett syndrome, secondary parkinsonism, spasticity, or Wilson's disease.
